(19) United States
(12) Patent Application Publication
(10) Pub. No.: US 2026/0002223 A1
(43) Pub. Date: Jan. 1, 2026

(54) UNIVERSAL METHOD FOR PARASITE AND EUKARYOTIC ENDOSYMBIONT IDENTIFICATION

(71) Applicant: Wisconsin Alumni Research Foundation, Madison, WI (US)

(72) Inventors: Tony GOLDBERG, Madison, WI (US); Leah OWENS, Madison, WI (US)

(21) Appl. No.: 18/879,241

(22) PCT Filed: Jun. 27, 2023
(86) PCT No.: PCT/US2023/069177

§ 371 Date: Dec. 26, 2024

Related U.S. Application Data
(60) Provisional application No. 63/355,931, filed on Jun. 27, 2022.

(51) Int. Cl. C12Q 1/6888

(52) U.S. Cl. CPC C12Q 1/6888

(57) ABSTRACT

The present invention provides methods for detecting eukaryotic endosymbionts in a sample. Primer sets, guide RNAs, and mock communities of eukaryotic endosymbionts for use in these methods are also provided. Further, methods for diagnosing and treating a subject with a parasitic infection are also provided.

### CROSS-REFERENCE TO RELATED APPLICATIONS

This application claims priority to U.S. Provisional Application No. 63/355,931, filed on Jun. 27, 2022, the contents of which are incorporated by reference in their entireties.

### STATEMENT REGARDING FEDERALLY SPONSORED RESEARCH

This invention was made with government support under grants AG049395 awarded by the National Institutes of Health. The government has certain rights in the invention.

### SEQUENCE LISTING STATEMENT

This application includes a sequence listing in XML format titled “960296.04426_ST26.xml”, which is 73,899 bytes in size and was created on Jun. 21, 2023. The sequence listing is electronically submitted with this application via Patent Center and is incorporated herein by reference in its entirety.

### BACKGROUND

Microbiome research has led to an explosion of knowledge about prokaryotic communities and their influence on host health. However, a parallel revolution for eukaryotic communities has yet to be realized. The field of parasitology, in particular, would benefit from a sequencing-based method for simultaneously characterizing multiple eukaryotic endosymbionts in complex clinical specimens. Currently, parasite diagnostics consist principally of microscopy and single-target assays such as polymerase chain reaction (PCR) and antigen-based tests for known agents. Thus, the parasite diagnostic process is laborious and expensive, and, in the case of microscopy, it relies on the presence of visible organisms or their eggs. Several metabarcoding-based approaches have been developed for parasite detection, but all have serious drawbacks, including lack of parasite taxonomic coverage and high levels of interfering host signal. Accordingly, there remains a need in the art for improved methods for parasite detection.

### SUMMARY

In a first aspect, the present invention provides primer sets for amplifying the V4 region of 18S ribosomal RNA (rRNA) genes found in eukaryotic endosymbionts. The primer sets comprise a forward primer comprising a sequence selected from SEQ ID NOs: 1-4 and a reverse primer comprising a sequence selected from SEQ ID NOs: 5-8.

In a second aspect, the present invention provides guide RNAs (gRNAs) that target the V4 region of 18S rRNA genes found in vertebrate organisms. The gRNAs are selected from SEQ ID NOs: 9-14.

In a third aspect, the present invention provides mock communities of eukaryotic endosymbionts comprising 18S rRNA genes, or portions thereof, from a plurality of eukaryotic endosymbionts in equimolar quantities. The plurality of eukaryotic endosymbionts comprises two or more eukaryotic endosymbionts selected from the group consisting of: Echinorhynchus salmonis (ES201), Hymenolopis diminuta (HD1), Ascaris suum (AS1), Dirofilaria immitis (DI8), Trichinella spiralis (TS3), Encephalitozoon cuniculi (EC2), Entamoeba histolytica (EH3), Balamuthia mandrillaris (BM2), Naegleria fowleri (NF12), Leishmania major (LM4), Giardia intestinalis (GI405), Plasmodium falciparum: (PF115), Babesia sp. (Bab10), Toxoplasma gondii (TG3), Cryptosporidium hominis (CH109), and Blastocystis hominis 1 (ATCC 50177) (BH1).

In a fourth aspect, the present invention provides methods for assessing the ability of a primer set to detect one or more eukaryotic endosymbionts. The methods comprise (a) amplifying a mock community described herein using the primer set; and (b) detecting any resulting amplicons. In these methods, detection of an amplicon associated with a particular eukaryotic endosymbiont (i.e., an amplicon comprising a genomic DNA sequence specific to that eukaryotic endosymbiont) indicates that the primer set is able to detect that particular eukaryotic endosymbiont.

In a fifth aspect, the present invention provides methods for detecting one or more eukaryotic endosymbionts in a sample. The methods comprise (a) extracting DNA from the sample; (b) amplifying the DNA using a primer set described herein to generate amplicons; (c) sequencing the amplicons to generate sequencing reads; and (d) analyzing the sequencing reads. In these methods, the presence of sequencing reads associated with a particular eukaryotic endosymbiont (i.e., sequencing reads that map to the 18S rRNA gene from that eukaryotic endosymbiont) indicates that that eukaryotic endosymbiont is present in the sample.

In a sixth aspect, the present invention provides methods for diagnosing and treating a subject with a parasitic infection. The methods comprise: (a) obtaining a sample from the subject; (b) extracting DNA from the sample; (c) amplifying the DNA using a primer set described herein to generate amplicons; (e) sequencing the amplicons to generate sequencing reads; (e) analyzing the sequencing reads to detect the presence of a parasite in the sample; and (f) treating the subject for the detected parasite.

In a seventh aspect, the present invention provides kits comprising one or more primer sets described herein and instructions for use. In some embodiments, the kits further comprise a gRNA described herein (i.e., to allow for host DNA depletion) and/or a mock community described herein (i.e., to allow for standardization across experiments and sample types).

### BRIEF DESCRIPTION OF THE DRAWINGS

FIGS. 1A-1E show development and evaluation of a new eukaryotic endosymbiont metabarcoding method, termed VESPA (Vertebrate Eukaryotic endoSymbiont and Parasite Analysis). FIG. 1A—Histogram of marker genes identified in a literature review of 54 host-associated eukaryotic endosymbiont studies. FIG. 1B—Primer sets for amplifying the 18S rRNA gene identified in our literature review, shown as a histogram binned by location along the 18S gene. Hypervariable regions V4 and V9 are demarcated by arrows below the x-axis. FIG. 1C—Generalized map of a eukaryotic 18S rRNA gene with hypervariable regions represented as open arrows labeled V2-V9. Newly designed and published metabarcoding primer sets are shown as colored arrows and the boxed areas 1-3 are expanded as insets. See Table 5 for full primer names and sequences. FIG. 1D—Heat map of published and new 18S V4 primer set coverage across clades exclusively containing parasites of vertebrates. Percent overall complementarity (% coverage) is shown as numbers and as a color scale (color key below heatmap; higher percentage represents lighter color) with taxonomic labels to the left. Red boxes highlight clades with low overall (“problematic”) coverage. FIG. 1E—Vertebrate endoparasite PCR panel showing amplification (+) or lack of amplification (−) of single-organism genomic DNA templates across new and published primer sets. Total represents the number of successful amplifications per primer out of 22 possible, shown in left-most “Theoretical” column. Hadz., Hadziavdic. Box highlights a clade with low overall (“problematic”) amplification.

FIGS. 2A-2E show testing of metabarcoding methods for amplification bias using a community standard. FIG. 2A—Schematic overview of EukMix creation via 18S isolation and cloning. FIG. 2B—Equimolar EukMix community standard metabarcoding across primer sets as compared to theoretical input (leftmost bar, blue box) shown as % abundance of reads per organism. The legend is in the same order as the abundance indication. FIG. 2C—Equimolar EukMix community standard metabarcoding reads assigned to each component organism shown as mean % abundance of three replicates+/−standard error of the mean (SEM) with theoretical input level of 6.25% displayed as a horizontal line. ES, Echinorhynchus salmonis; HD, Hymenolepis diminuta; AS, Ascaris suum; DI, Dirofilaria immitis; TS, Trichinella spiralis; EC, Encephalitozoon cuniculi; EH, Entamoeba histolytica; BM, Balamuthia mandrillaris, NF, Naegleria fowleri; LM, Leishmania major; GI, Giardia intestinalis, PF, Plasmodium falciparum; Bab, Babesia sp. strain MO1; TG, Toxoplasma gondii; CH, Cryptosporidium hominis; BH, Blastocystis hominis. See Table 6 for parasite sources and strains. FIG. 2D—Mean absolute distance to the theoretical input level for each primer set for three replicates+/−SEM. P values are derived from two-tailed Wilcoxon matched-pairs signed rank tests. Owens 29F was significantly different from all other primer sets (shown as bars with asterisks). All comparisons not shown are not significant. FIG. 2E—Diversity metrics based on EukMix analysis compared to theoretically equal input (shaded row). Primer set 29F represented the underlying community most accurately by all three metrics (bolded row).

FIGS. 3A-3F show a comparison of VESPA and microscopy using human clinical samples. FIGS. 3A-3C show VESPA metabarcoding data. VESPA data are shown as percent relative abundance of each organism category with all quality-filtered reads included (FIG. 3A), with helminth reads only (FIG. 3B), or with protozoal reads only (FIG. 3C) (archaea, bacteria, host, plants, invertebrates, and fungi are removed in FIG. 3B and FIG. 3C). In FIG. 3A, the numbers above bars are the total percentage of prokaryotic (bacterial+archaeal) reads. FIG. 3D—Microscopy versus VESPA. Microscopy findings (M) are shown as a presence/absence (Y=present, N=absent, NA=not assessed) and VESPA metabarcoding (MB) findings are shown as % abundance of quality-filtered reads. Blue cells represent detection by VESPA, green cells by both VESPA and microscopy, and white cells by neither method. No organisms were identified by microscopy alone. Richness (final 2 rows, shaded cells) is defined as the total number of species detected by the specified method. Prevalence (final 2 columns, shaded cells) is defined as the proportion of the population positive for an organism by the specified method. Note that Onchocerca is not detectable in fecal samples by microscopy (asterisk). FIG. 3E—Proportional Venn diagrams of findings by microscopy versus VESPA. Individuals identified as positive for the listed organisms by VESPA (blue) or both (green) are shown as numbers in each circle. Overall findings summed over all organisms are shown to the left of the bracket (not to scale). Note that Onchocerca is not detectable in fecal samples by microscopy (asterisk). FIG. 3F—Richness and prevalence calculations for microscopy (M) and VESPA metabarcoding (MB) findings. Data are shown as mean+/−SEM. P values are derived from Wilcoxon matched-pairs signed rank tests, 2-tailed. ns, not significant.

FIGS. 4A-4F show a comparison of VESPA and microscopy using non-human primate clinical samples. FIGS. 4A-4C show VESPA metabarcoding data. VESPA data are shown as percent relative abundance of each organism category with all quality-filtered reads included (FIG. 4A), with helminth reads only (FIG. 4B; top bar in FIG. 4A), or with protozoal reads only (FIG. 4C; second to top bar in FIG. 4A) (archaea, bacteria, host, plants, invertebrates, and fungi are removed in FIG. 4B and FIG. 4C). In FIG. 4A, the numbers above bars are the total percentage of prokaryotic (bacterial+archaeal) reads. In FIG. 4C, the asterisk indicates a microsporidian parasite. FIG. 4D—Microscopy versus VESPA. Microscopy findings (M) are shown as a qualitative score (1 least-3 most) for protozoa, larvae/gram feces for Strongyloides, and eggs/gram feces for all other helminths. VESPA findings (MB) are shown as % abundance of quality-filtered reads. Yellow cells represent parasite detection by microscopy, blue cells by VESPA, green cells by both methods, and white cells by neither method. Richness (final 2 rows, shaded cells) is defined as the total number of species detected by the specified method. Prevalence (final 2 columns, shaded cells) is defined as the proportion of the population positive for an organism by the specified method. Note that Entamoeba histolytica and Entamoeba dispar are a cryptic species complex that cannot be resolved by microscopy (asterisk) and Piroplasmida sp. are not detectable in fecal samples by microscopy (double asterisk). FIG. 4E—Proportional Venn diagrams of findings by microscopy versus VESPA. Individuals identified as positive for the listed organisms by microscopy (yellow), VESPA (blue), or both (green) are shown as numbers in each circle. Overall findings summed over all organisms are shown to the left of the bracket (not to scale). Note that Entamoeba histolytica and Entamoeba dispar are a cryptic species complex that cannot be resolved by microscopy (asterisk) and Piroplasmida sp. are not detectable in fecal samples by microscopy (double asterisk). FIG. 4F—Richness and prevalence calculations for microscopy (M) and VESPA (MB) findings. Data are shown as mean+/−SEM. P values are derived from Wilcoxon matched-pairs signed rank tests, 2-tailed. ns, not significant. NA, not applicable (single data point only).

FIG. 5 shows a comparison of the VESPA primers. Three of the four forward primers are almost entirely overlapping (i.e., 9F, 13F, 2-2bF) and one overlaps with 9 of 18 positions (i.e., 29F). The four reverse primers all contain the same base sequence (i.e., 21b8R) with 4, 5, or 6 additional bases added for compatibility with forward primer melting temperatures (i.e., 21b8R+4, 21b8R+5, and 21b8R+6). Note that the reverse primers are shown in the reverse complement orientation in this figure such that all the primer sequences are going in the same direction.

FIG. 6 demonstrates that certain primer/DNA polymerase combinations work best (i.e., maximize target eukaryotic reads and minimize off-target prokaryotic reads) for amplification of DNA from fecal samples. The percent abundance after quality filtering is shown for target (lighter color green) and off-target (gray) reads.

FIGS. 7A-7B show 18S metabarcoding with peptide nucleic acid (PNA) mammal blocker in nonhuman primate samples. FIG. 7A—Percent relative abundance after quality filtering is shown for host reads (Host) and all other reads (Other). Numbers above bars represent percentage abundance of host reads. FIG. 7B—Mean relative abundance after quality filtering +/−SEM is shown for host reads (Host) and all other reads (Other). See Table 12 for source data.

FIGS. 8A-8C show an overview of the CRISPR-Cas9 host digestion method. FIG. 8A—Schematic depiction of CRISPR-Cas9 in vitro digestion of host amplicons. FIG. 8B—Map of representative mammal 18S rRNA gene (green region) from the house mouse (Mus musculus; GenBank NR_003278) with locations of 18S amplicon primers (black arrows), newly designed guide RNA (gRNA) sequences (yellow arrows), and published PNA mammal blocker (white arrow). Protospacer adjacent motifs (PAMs) within the host 18S sequence are shown in pink. gRNAs must bind next to a PAM sequence, and binding determines the location of cleavage by the Cas9 ribonucleoprotein complex. FIG. 8C—Schematic of digestion products of mouse 18S V4 amplicons using gRNAs to target various sites. Topmost fragment (no digest) is the full-length host amplicon. Labels to the left are gRNA names. See Table 13 for gRNA and PAM sequences.

FIG. 9 shows gRNA complementarity to host and eukaryotic endosymbiont groups. Percent coverage of the SILVA 138 Ref NR database is shown with numbers and color scale. Left panel—SILVA TestProbe with the most stringent settings (no mismatches, no N's considered as matches). Right panel—SILVA TestProbe allowing for a single mismatch outside of the conserved seed sequence. Taxonomic groups containing non-target “Host” groups and target “Eukaryotic endosymbiont” groups are shown with representative organism icons to the left of the heatmap. Tetrapoda includes the “Host” groups Amphibia, Aves, Crocodylia, Lepidosauria, Mammalia, and Testudines. Nematoda includes all nematode accessions other than Trichinella pseudospiralis. See Table 13 for gRNA sequences.

FIG. 10 shows in vitro CRISPR-Cas9 digests of host and eukaryotic endosymbiont 18S V4 amplicons. Gel electrophoresis images show CRISPR-Cas9 digestion products of 18S V4 DNA amplified from vertebrate hosts (left panel) and eukaryotic endosymbiotic organisms (right panel) with the name of the gRNA in the center. Sources of substrate DNA are shown as organism icons. Black icons represent organisms not cleaved by CRISPR-Cas9 digest with the specified gRNA, and gray icons represent organisms cleaved by CRISPR-Cas9 with the specified gRNA. Organisms used for digest were: Mammalia—Ursus maritimus (polar bear), Amphibia—Lithobates chiricahuensis (leopard frog), Aves—Gallus gallus (chicken), Lepidosauria—Varanus varius (monitor lizard), Neopterygii—Salmo trutta (brown trout), Amoebazoa—Entamoeba histolytica, Excavata—Trypanosoma brucei, Microsporidia—Encephalitozoon cuniculi, Acanthocephala—Echinorhynchus salmonis, Platyhelminthes—Schistosoma mansoni, and Nematoda—Ascaris suum. Topmost row is a DNA size standard. Note that 18S V4 amplicon length is variable among eukaryotic endosymbionts and that no eukaryotic endosymbiont amplicons were digested using any of the gRNAs tested.

FIGS. 11A-11B show a comparison of host signal reduction with mammal blocking PNA oligo compared to with CRISPR-Cas9 amplicon digestion in 18S V4 metabarcoding. FIG. 11A—Percent abundance of host reads after quality filtering for five DNA samples metabarcoded under four conditions (triplicate mean): no host signal reduction used (None), published mammal-blocking PNA oligo added to amplicon PCR (PNA), CRISPR-Cas9 digest of amplicons (CRISPR-Cas9), and mammal-blocking PNA oligo added to amplicon PCR plus subsequent CRISPR-Cas9 digest of amplicons (PNA+CRISPR-Cas9). Note the scale difference in tissues versus fecal sample. FIG. 11B—Results from FIG. 11A displayed as percent change in target (non-host) read abundance as compared to no-treatment control for all non-fecal samples. PNA, published mammal-blocking PNA oligo added to amplicon PCR; CC9, CRISPR-Cas9 digest of amplicons; Both, mammal-blocking PNA oligo added to amplicon PCR plus subsequent CRISPR-Cas9 digest of amplicons. CRISPR-Cas9 treatment is significantly different from PNA (paired t-test: t=6.94, df=3, P=0.0061) and Both (paired t-test: t=8.89, df=3, P=0.0030). See Table 14 for source data.

FIGS. 12A-12E show characterization and optimization of CRISPR-Cas9 mediated host signal reduction in 18S V4 metabarcoding. FIG. 12A—CRISPR-Cas9 (CC9) reaction optimization. Percent host read abundance (triplicate mean+/−SEM) after quality filtering using varying ribonucleoprotein complex (RNP) to DNA target sequence ratios, where 1× represents a 1:1 ratio. FIG. 12B—Identity of high and low molecular weight (MW) CC9 cleavage products. Percent host read abundance (triplicate mean+/−SEM) after quality filtering is shown for high and low MW bands extracted after separation by gel electrophoresis. FIG. 12C—Comparison of CC9 digest before and after indexing PCR. Mean percent host read abundance+/−SEM after quality filtering is shown for CC9 digest applied to each amplicon prior to library preparation (Not pooled) or to a single pool of amplicons after library preparation (Pooled). ns, not significant (paired t-test: t=1.38, df=30, P=0.18). FIG. 12D—Effect of gRNA sequence on blood sample 18S V4 metabarcoding. Percent host read abundance (triplicate mean+/−SEM) after quality filtering is shown for 18S V4 amplicons that were not treated with any host signal reduction method (None) or digested with CRISPR-Cas9 using the specified gRNA prior to library preparation. See Table 15 for source data. FIG. 12E—Comparison of gRNAs in blood sample metabarcoding. Mean percent host reads abundance+/−SEM after quality filtering is shown for three guide RNAs compared to no digest control. *P<0.05, ****P<0.0001, all comparisons not shown are insignificant (paired t-test, df=30 in all comparisons). See Table 15 for source data.

FIGS. 13A-13B show the effect of host signal reduction method on detection of a known parasite infection. Dog blood infected with Dirofilaria immitis microfilariae was used as starting material for DNA extraction and 18S metabarcoding. Amplicons were untreated for host signal reduction (None), amplified with a PNA mammal blocker (PNA), or digested with CRISPR-Cas9 using the specified gRNA (X axis). Percent abundance after quality filtering is shown for all filtered reads (FIG. 13A) or reads after removing host sequences (FIG. 13B). Numbers above bars represent total percentage host reads (FIG. 13A) or total percentage D. immitis reads (FIG. 13B). Note difference in scale between FIG. 13A and FIG. 13B. See Table 16 for source data.

FIG. 14 shows the effect of CRISPR-Cas9 host signal reduction on detection of hemoparasite infection in wild non-human primate blood samples. Metabarcoding data are shown as percent read abundance after quality filtering for undigested (left panel) and CRISPR-Cas9 digested (right panel) amplicons using 19 samples. Reads are categorized as host, Hepatocystis spp., and all other reads (Other). Numbers above bars represent total % host reads per sample. Legend shows the order of the bars. No Hepatocystis spp. positives were detected by metabarcoding in undigested samples. See Table 17 for source data.

FIG. 15A-15B show the results of performing 18S V9 metabarcoding on non-human primate fecal samples using the Earth Microbiome Project (EMP) protocol. The percent relative abundance of each organism category is shown with all quality-filtered reads included (FIG. 15A) and with parasite reads only (FIG. 15B) (i.e., archaea, bacteria, environmental sequences, plants, and fungi removed). Note: Sample OI-3 had zero parasite reads and is therefore not included in FIG. 15B. The legends show the order of the bars.

FIG. 16 shows the results of a parasite assessment of non-human primate fecal samples by microscopy and 18S V9 metabarcoding using the EMP protocol. Microscopy findings (M) are shown as a qualitative score (1 least-3 most) for protozoa, larvae/gram feces for Strongyloides, and eggs/gram feces for all other helminths. Metabarcoding findings (MB) are shown as % abundance of quality-filtered reads. Light gray cells represent parasite detection by microscopy, medium gray cells represent parasite detection by metabarcoding, black cells represent parasite detection by both methods, and white cells represent parasite detection by neither method. Richness (final 2 rows, shaded cells) is defined as the total number of species detected by the specified method.

FIG. 17 shows the results of performing 18S V9 metabarcoding using the EMP protocol on human fecal samples. The percent relative abundance after quality filtering is shown for each organism category detected. Legend shows the order of the bars from top to bottom.

FIG. 18 shows the results of a parasite assessment of human fecal samples by microscopy and 18S V9 metabarcoding using the EMP protocol. Microscopy findings (M) are shown as larvae/gram feces for Strongyloides and eggs/gram feces for all other helminths. Metabarcoding findings (MB) are shown as % abundance of quality-filtered reads. Shaded cells represent parasite detection by microscopy and white cells represent parasite detection by neither microscopy nor metabarcoding. Richness (final row, shaded cells) is defined as the total number of species detected by the specified method.

FIG. 19A-19B show a comparison of the results of 18S V4 and 18S V9 metabarcoding of non-human primate fecal samples. The percent relative abundance of each organism category is shown with all quality-filtered reads included (FIG. 19A) and with reads from archaea, bacteria, and the host organism as well as uncultured environmental reads removed (FIG. 19B). The numbers above the bars in FIG. 19B represent the percentage of reads mapping to helminth organisms. Legends shows the order of the bars from top left to bottom right.

### DETAILED DESCRIPTION

The present invention provides methods for detecting eukaryotic endosymbionts in a sample. Primer sets, guide RNAs, and mock communities of eukaryotic endosymbionts for use in these methods are also provided. Further, methods for diagnosing and treating a subject with a parasitic infection are also provided.

The methods of the present invention are methods for metabarcoding eukaryotic endosymbionts, including eukaryotic endoparasites. “Metabarcoding,” also referred to as metagenomic barcoding, is a method in which organisms present in a complex sample are identified via amplification and sequencing of a specific portion of a gene that is conserved across the targeted organisms. The resulting sequencing reads are quality-filtered, de-replicated, and compared with reference databases to assign taxonomic identifications. Thus, the metabarcoding methods allow for the simultaneous identification of many taxa of eukaryotic endosymbionts within the same sample.

As is described in Example 1, the inventors developed their methods after obtaining unsatisfactory results with two published eukaryotic endosymbiont metabarcoding protocols. The published methods did not detect all eukaryotic endosymbionts present in the samples, and the vast majority (>90%) of reads produced by these methods were of bacterial or host origin. Like the published metabarcoding protocols, the inventors' method, which they have named VESPA (Vertebrate Eukaryotic endoSymbiont and Parasite Analysis), involves amplifying a target gene and sequencing the resulting amplicons. However, their method utilizes improved polymerase chain reaction (PCR) primers that were designed to amplify the V4 region of the 18S rRNA (18S) gene from all eukaryotic endosymbionts. When tested in silico, the inventors' primers amplified DNA from parasites from all 24 clinically relevant parasite clades. In contrast, the primers utilized in the published metabarcoding protocols amplified DNA from an average of only 15.7 of these clades.

To validate their primers in vitro, the inventors generated a mock community of eukaryotic endosymbionts. Although there are mock communities of bacteria and yeasts available for standardizing microbiome assays, no equivalent for eukaryotic endosymbionts is commercially available. Thus, the inventors cloned full-length 18S genes from 16 parasitic organisms and combined them in equimolar ratios to create a mock community that is referred to herein as “EukMix”. The inventors used this reagent to directly compare the accuracy of their primer sets to that of several high-performing published primer sets. They found that only their primer sets were able to detect all 16 18S genes included in EukMix and that they generated sequencing data that better represented the relative abundances of the underlying eukaryotic endosymbiont community. Additionally, they tested their primer sets against the same published primer sets using fecal samples to assess off-target amplification of bacterial sequences. They found that bacterial read abundance in the data generated using their primers was dramatically lower than in the data generated using the published primers.

In sample types other than feces, most interfering reads are of host origin because primers designed to amplify all eukaryotic endosymbionts will also amplify eukaryotic host DNA. Thus, as is described in Example 3, the inventors developed a method for reducing host signal using CRISPR-Cas9 in vitro digestion. In this method, Cas9 nuclease is targeted to host-specific 18S gene sequences via one of six guide RNAs (gRNAs), and the resulting fragments are purified away by size selection prior to sequencing. Using in silico hybridization, they found that their gRNAs did not recognize 18S genes from eukaryotic endosymbionts (with the exception of one nematode worm) but did recognize 18S genes from mammals and other vertebrates. The inventors then tested their gRNAs by performing CRISPR-Cas9 digestion of a panel of genomic DNA isolates, and demonstrated that 18S amplicons from mammals, birds, amphibians, reptiles, and fish are digested by this method while 18S amplicons from eukaryotic endosymbionts remain intact. They compared the efficacy of their method for eukaryotic endosymbiont detection in blood and tissue samples to that of a published protocol that utilizes mammal blocking oligos and found that CRISPR-Cas9 digestion resulted in fewer interfering host reads than the blocking oligo treatment.

Together, this work demonstrates the potential of the inventors' metagenomic barcoding method for identifying eukaryotic endosymbionts in a wide range of host and sample types. Their method is “universal,” in that it can detect all eukaryotic endosymbionts simultaneously, whether or not they are visible microscopically. It is also faster, more accurate, more comprehensive, and ultimately has a lower “cost to answer” than any prior method. The inventors envision that this method will be useful for both clinical/veterinary parasite diagnosis and for research on eukaryotic endosymbiont communities in hosts and the environment.

### Primers:

In a first aspect, the present invention provides primer sets for amplifying the V4 region of the 18S genes found in eukaryotic endosymbionts. The primer sets comprise a forward primer comprising a sequence selected from SEQ ID NOs: 1-4 and a reverse primer comprising a sequence selected from SEQ ID NOs: 5-8. In Examples 1 and 2, the inventors describe the generation of these primer sets and demonstrate that (1) they can be used to detect parasites from all clinically relevant parasite clades, and (2) that they produce far less off-target amplification than previously published primer sets.

As used herein, a “primer” is a single-stranded DNA oligonucleotide designed to bind to a sequence within or flanking a DNA target sequence via complementary base pairing. DNA polymerases are only capable of adding nucleotides to the 3′-end of an existing nucleic acid. Thus, the binding of a primer to a DNA template strand creates a site from which DNA polymerase can initiate synthesis of a complementary DNA strand in an amplification reaction. Primers can be chemically synthesized or ordered from commercial vendors.

The primers of the present invention are designed for use in a DNA amplification method. Two primers, i.e., a forward primer and reverse primer, are required for DNA amplification, one for each of the complementary strands of the DNA target sequence. The “forward primer” binds to the anti-sense strand on the 5′ end of the target sequence, while the “reverse primer” binds to the sense strand on the 3′ end of the target sequence. Thus, the 5′ ends of the primers define the termini of the amplified DNA target sequence (i.e., the amplicon). A pair of a forward primer and a reverse primer that is designed to amplify a DNA target sequence is referred to as a “primer set”.

In some embodiments, the primers further comprise adapters. Specifically, in some embodiments, the forward primer further comprises a first adapter and the reverse primer further comprises a second adapter. Adapters can be added to primers as demonstrated in the Examples (see, e.g., the VESPA Protocol in Example 1).

As used herein, an “adapter” is a DNA sequence designed to interact with a specific sequencing platform to facilitate a sequencing reaction. For example, in the Illumina sequencing workflow, the adapters contain complementary sequences that allow the DNA fragments to bind to the flow cell. The optimal length of an adapter will vary depending on the sequencing platform used. One of ordinary skill will understand that adapter sequences may be as short as 20 nucleotides or substantially longer. Examples of suitable adapter sequences are disclosed herein as SEQ ID NO: 66 and SEQ ID NO: 67. In some embodiments, the sequencing adapters comprise unique molecular identifier (UMI) sequences, which comprise a sequence label (e.g., a random DNA sequence) that is unique to each DNA molecule to enable its quantification and identification. In some embodiments, the sequencing adapters comprise “barcode” sequences, which are used to label all DNA molecules from a particular sample or source (e.g., DNA from a particular tissue, subject, organism, or environment). The inclusion of barcodes in the adapters allows multiple sequencing libraries to be sequenced simultaneously during a single run, thereby reducing sequencing costs.

### Guide RNAs:

In a second aspect, the present invention provides guide RNAs (gRNAs) that target the V4 region of 18S rRNA genes found in vertebrate organisms. These gRNAs are disclosed as SEQ ID NOs: 9-14. In Example 3, the inventors describe the generation of these gRNAs and demonstrate that they can be used to specifically deplete vertebrate 18S sequences via CRISPR-Cas9 digestion. Thus, these gRNAs can be used to increase the efficacy of eukaryotic endosymbiont detection methods by depleting DNA sequences from the host organism.

A “guide RNA (gRNA)” is a single-stranded RNA oligonucleotide that recruits an RNA-guided nuclease to a specific genomic sequence via complementary base pairing.

In some embodiments, the gRNAs are chemically modified. For example, the gRNAs may be chemically modified to decrease a cell's ability to degrade them. Chemically modified gRNAs may include one or more of the following exemplary modifications or others available to those skilled in the art: 2′-fluoro (2′-F), 2′-O-methyl (2′-OMe), S-constrained ethyl (cEt), 2′-O-methyl (M), 2′-O-methyl-3′-phosphorothioate (MS), and 2′-O-methyl-3′-thiophosphonoacetate (MSP).

The sequences of ribosomal RNA (rRNA) genes, including 16S rRNA, 18S rRNA, and 23S rRNA, are commonly used to identify microorganisms present within a sample since they are found across nearly all forms of life. As is noted above, the primers and gRNAs described herein are designed to target a portion of the small subunit 18S rRNA (18S) gene, which is one of the most commonly used markers for taxonomic identification in eukaryotes.

In Example 3, the inventors demonstrate that the six disclosed gRNAs hybridize to over 50% of mammalian 18S rRNA gene amplicons but fail to hybridize to eukaryotic endosymbiont 18S rRNA gene amplicons. As used herein, the term “amplicon” refers to an amplification product. Thus, an “18S rRNA gene amplicon” is an amplification product generated using an 18S rRNA gene or portion thereof as the DNA template.

The six gRNAs have different specificities. While arb321 (SEQ ID NO: 9) and arb326 (SEQ ID NO: 10) only hybridize to mammalian sequences; arb615 (SEQ ID NO: 11), CA149 (SEQ ID NO: 13), and CA172 (SEQ ID NO: 14) hybridize to mammal, bird, and fish sequences; and PT7.1 (SEQ ID NO: 12) hybridizes to sequences from all tested vertebrates. Thus, for effective vertebrate host DNA depletion, the gRNA should be selected based on the host from which a sample is derived.

As used herein, the term “hybridize” refers to the process in which two complementary single-stranded polynucleotides bind together to form a double-stranded molecule. The term “complementary” refers to the ability of a polynucleotide to bind to (i.e., hybridize with) another nucleic acid molecule through the formation of hydrogen bonds between specific nucleotides (i.e., A with T or U and G with C). Whereas PCR primers can still bind to partially complementary sequences to prime DNA synthesis under some conditions, gRNAs must exactly match an 8-10 base seed sequence within the target sequence to be able to bind to the active site of the nuclease. The target sequence must additionally contain a 3-5 base protospacer adjacent motif (PAM) to be recognized and cleaved by the nuclease, which further increases target specificity and limits the number of possible sites for gRNA targeting.

### Mock Communities:

In a third aspect, the present invention provides mock communities of eukaryotic endosymbionts. The mock communities comprise 18S rRNA genes, or portions thereof, from a plurality of eukaryotic endosymbionts in equimolar quantities. The plurality of eukaryotic endosymbionts comprises two or more eukaryotic endosymbionts selected from the group consisting of: Echinorhynchus salmonis (ES201), Hymenolopis diminuta (HD1), Ascaris suum (AS1), Dirofilaria immitis (DI8), Trichinella spiralis (TS3), Encephalitozoon cuniculi (EC2), Entamoeba histolytica (EH3), Balamuthia mandrillaris (BM2), Naegleria fowleri (NF12), Leishmania major (LM4), Giardia intestinalis (GI405), Plasmodium falciparum (PF115), Babesia sp. (Bab10), Toxoplasma gondii (TG3), Cryptosporidium hominis (CH109), and Blastocystis hominis 1 (ATCC 50177) (BH1).

As used herein, a “mock community” is a mixture of polynucleotides that was created in vitro to simulate the polynucleotides that would be isolated from a community of eukaryotic endosymbionts. The mock communities of the present invention may comprise nucleic acids from about 2-50 different eukaryotic endosymbionts, preferably from about 5-20 eukaryotic endosymbionts. In the Examples, the inventors combined nucleic acids from 16 different eukaryotic endosymbionts (i.e., ES201, HD1, AS1, DI8, TS3, EC2, EH3, BM2, NF12, LM4, GI405, PF115, Bab10, TG3, CH109, and BH1) to form the mock community referred to herein as “EukMix”. These 16 eukaryotic endosymbionts were selected such that they cover the taxonomic range of clinically important parasitic organisms (i.e., protozoan, worm, and microsporidian). Thus, in some embodiments, the mock community comprises polynucleotides from these 16 specific eukaryotic endosymbionts.

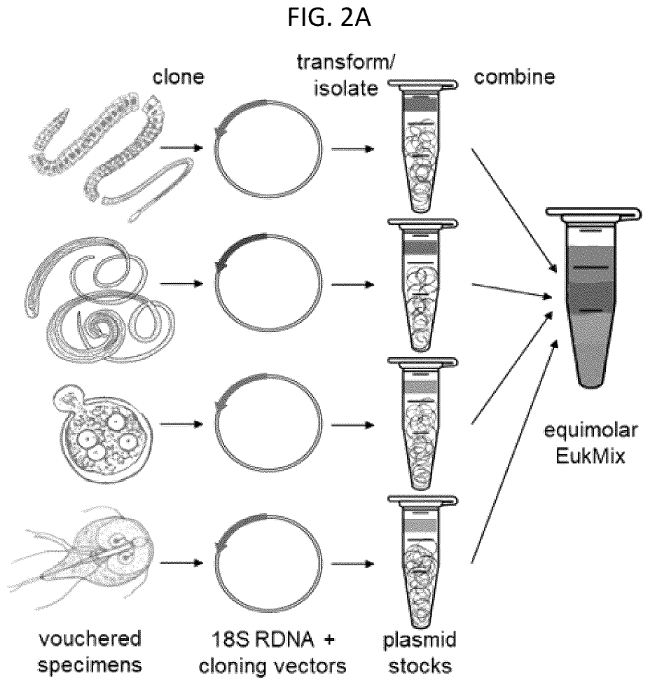

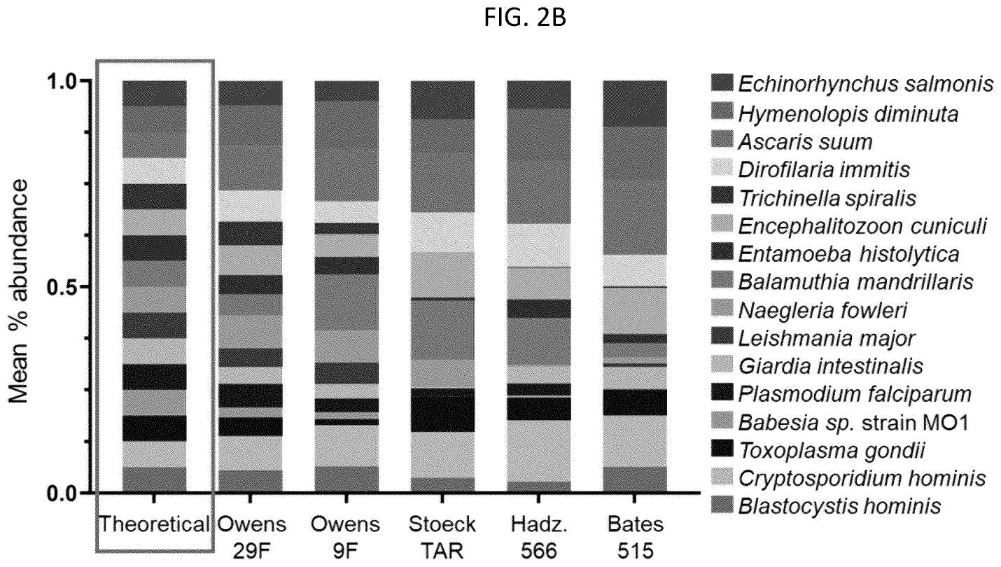

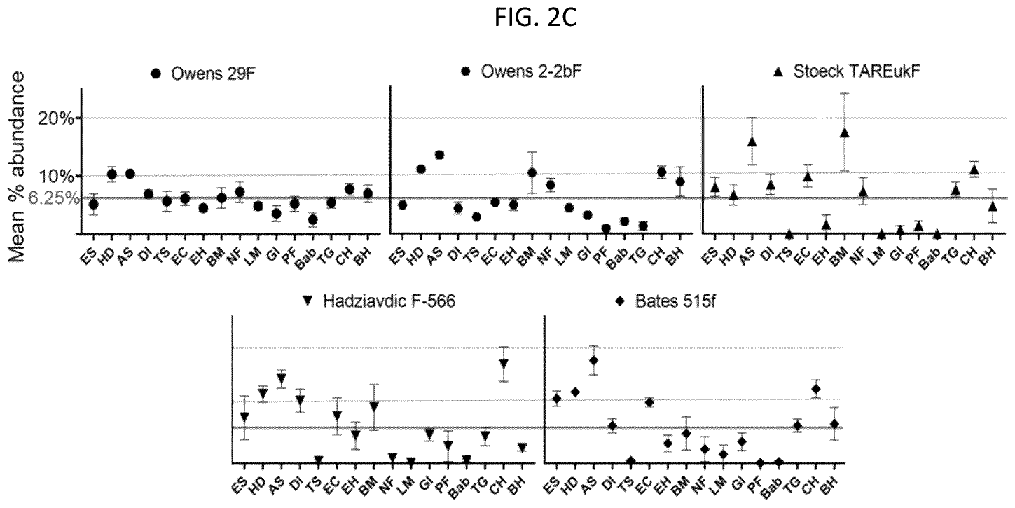

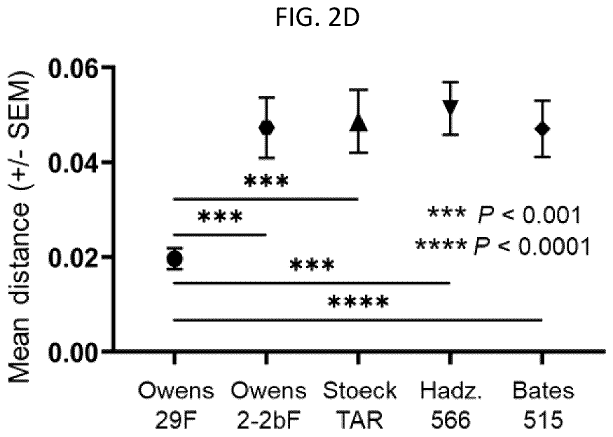

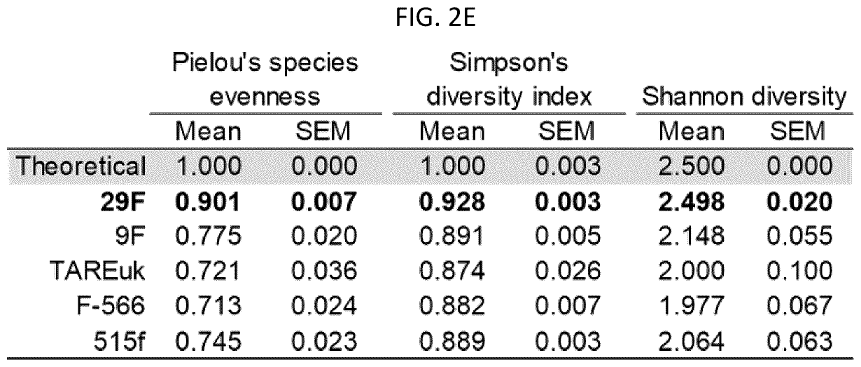

EukMix, which is depicted schematically in FIG. 2A, comprises an equimolar mix of 16 different plasmids, each of which contain the full-length 18S rRNA gene from one of the 16 eukaryotic endosymbionts noted above. Thus, in some embodiments, each 18S rRNA gene or portion thereof included in the mock community is provided as part of a plasmid. A “plasmid” is a small circular DNA molecule that can replicate independently from chromosomal DNA.

The mock communities disclosed herein can be used to assess the ability of a primer set to detect one or more eukaryotic endosymbionts, as described in the following section. Additionally, the mock communities disclosed herein can be used as a positive control (i.e., to allow for standardization across experiments and sample types) in the methods for detecting eukaryotic endosymbionts and the methods for treating parasitic infections discussed below.

### Methods for Assessing Primers:

In a fourth aspect, the present invention provides methods for assessing the ability of a primer set to detect one or more eukaryotic endosymbionts. The methods comprise (a) amplifying a mock community described herein using the primer set; and (b) detecting any resulting amplicons. In these methods, detection of an amplicon associated with a particular eukaryotic endosymbiont (i.e., an amplicon comprising a genomic DNA sequence specific to that eukaryotic endosymbiont) indicates that the primer set is able to detect that particular eukaryotic endosymbiont.

Notably, in embodiments in which a known amount of template DNA is used, these methods can also be used to assess the quantitative potential of the assay (i.e., by comparing the number of copies of template DNA to the number of reads produced therefrom).

The term “amplification” refers to a template-dependent process that results in an increase in the concentration of a DNA molecule relative to its initial concentration. A “template-dependent process” is a process in which the sequence of the newly synthesized DNA molecule is dictated by the rules of complementary base pairing. The amplification step of the present methods can be performed using any amplification method known in the art. Exemplary amplification methods include polymerase chain reaction (PCR), loop-mediated isothermal amplification (LAMP), strand displacement amplification (SDA), ligase chain reaction (LCR), and transcription-mediated-amplification (TMA). However, in preferred embodiments, the amplification step is performed using PCR.

PCR is an in vitro method used to selectively amplify a specific DNA target sequence in a sample. PCR employs two main reagents: primers and a DNA polymerase. In PCR, a repeated series of reaction steps (i.e., template denaturation, primer annealing, and extension of the annealed primers by DNA polymerase) results in exponential amplification of the target sequence. See Saiki et al., 1985, Science 230:1350 for a detailed description of PCR.

PCR is commonly performed using a “reaction mixture” that comprises template DNA (typically 1-1000 ng) and at least about 25 pmol of each primer. The reaction mixture must also include deoxynucleotide triphosphates (dNTPs) and a DNA polymerase. For example, a typical reaction mixture might include: 2 μl of template DNA, 25 pmol of each primer, 2.5 μl of a suitable buffer, 0.4 μl of 1.25 μM dNTP, 2.5 units of DNA polymerase, and deionized water to a total volume of 25 μl. The reaction mixture may include additional reagents such as a GC enhancer to increase amplification specificity. In the methods of the present invention, the template DNA is eukaryotic endosymbiont genomic DNA and the primers comprise a forward primer selected from SEQ ID NOs: 1-4 and a reverse primer selected from SEQ ID NOs: 5-8. The template DNA may also be derived from a sample obtained from a subject.

A “DNA polymerase” is an enzyme that catalyzes the polymerization of DNA. The polymerase initiates synthesis starting at the 3′-end of the primers annealed to the target sequence, and proceeds in the 5′-direction along the template DNA. Known DNA polymerases for use in PCR methods include, without limitation, E. coli DNA polymerase I, T7 DNA polymerase, Thermus thermophilus (Tth) DNA polymerase, Bacillus stearothermophilus DNA polymerase, Thermococcus litoralis DNA polymerase, Thermus aquaticus (Taq) DNA polymerase, and Pyrococcus furiosus (Pfu) DNA polymerase. Many suitable Taq polymerases are commercially available including, for example, HotStarTaq™ DNA Polymerase, Platinum™ II Taq Hot-Start DNA Polymerase, AmpliTaq™ DNA Polymerase, FastStart™ Taq DNA Polymerase, and TaKaRa Ex Taq™ DNA Polymerase. In the Examples, the inventors determined that the Platinum™ II Taq Hot-Start DNA Polymerase worked better than several other polymerases for amplification of eukaryotic endosymbiont 18S rRNA gene sequences from fecal samples. Thus, in some embodiments, the polymerase is Platinum™ II Taq Hot-Start DNA Polymerase.

PCR is performed using a programmable thermal cycler. The length and temperature of each step of a PCR cycle, as well as the number of cycles, are adjusted according to the stringency requirements of the particular reaction. Annealing temperature and timing are determined both by the efficiency with which a primer is expected to anneal to the template DNA and by the degree of mismatch (i.e., between the primer and template DNA) that can be tolerated. The ability to optimize the stringency of primer annealing conditions is well within the knowledge of one of ordinary skill in the art. An annealing temperature of between 30° C. and 72° C. is typically used. An initial denaturation of the template molecules is normally performed for a period of time (e.g., for 4 minutes) at between 92° C. and 990 C, followed by 20-40 cycles consisting of a denaturation step (94-99° C. for 15 seconds to 1 minute), annealing step (temperature determined as discussed above; 30 seconds-2 minutes), and extension step (72° C. for 1 minute). An optional final extension step is generally carried out for 4 minutes at 72° C., and may be followed by an indefinite (0-24 hour) hold at 4° C.

Following amplification, any resulting amplicon is detected. Detection may be performed using any method known in the art. Suitable methods for detecting an amplicon include, without limitation, gel electrophoresis, sequencing (e.g., Sanger sequencing, single-molecule sequencing, high-throughput sequencing, pyrosequencing), restriction fragment length polymorphism (RFLP) analysis, and quantitative PCR (qPCR).

### Methods for Detecting Eukaryotic Endosymbionts:

In a fifth aspect, the present invention provides methods for detecting one or more eukaryotic endosymbionts in a sample. The methods comprise (a) extracting DNA from the sample; (b) amplifying the DNA using a primer set described herein to generate amplicons; (c) sequencing the amplicons to generate sequencing reads; and (d) analyzing the sequencing reads. In these methods, the presence of sequencing reads associated with a particular eukaryotic endosymbiont (i.e., sequencing reads that map to the 18S rRNA gene from that eukaryotic endosymbiont) indicates that the eukaryotic endosymbiont is present in the sample.

The methods of the present invention allow for detection of eukaryotic endosymbionts. As used herein, the term “eukaryotic endosymbionts” includes all non-fungal eukaryotes residing within vertebrate hosts. This term includes both microscopic eukaryotes (e.g., microsporidia, protozoa, algal parasites) and macroscopic metazoans (e.g., helminths, pentastomes). The prefix “endo” is meant include endoparasites and commensals, while excluding ectoparasites (e.g., mites, ticks, fleas).

As noted above, the term eukaryotic endosymbiont encompasses eukaryotic endoparasites. A “parasite” is an organism that lives in or on an organism of another species (its host) and derives benefits at the other organism's expense. Eukaryotic endoparasites include protozoans, helminths (worms), and microsporidians. Thus, in some embodiments, the methods of the present invention are used to detect parasites or diagnose parasitic diseases.

In step (a) of the present methods, DNA is extracted from the sample. “DNA extraction” is a process in which DNA is separated from cell membranes, proteins, and other cellular components using physical and/or chemical methods. DNA can be extracted using various methods that are well known in the art, including those that rely on organic extraction, ethanol precipitation, silica-binding chemistry, cellulose-binding chemistry, and ion exchange chemistry. Many reagents and kits for DNA extraction are commercially available.

In step (b), the DNA is amplified using a primer set described herein. As is discussed above, any method of DNA amplification may be used with the present methods. However, in preferred embodiments, the DNA is amplified via PCR.

In step (c), the amplicons are sequenced to generate sequencing reads. DNA sequencing is the process of determining the order of nucleotides in a DNA molecule. Any DNA sequencing method may be used with the present invention. Suitable methods include, for example, Sanger sequencing, Illumina sequencing, single molecule real time (SMRT) sequencing, Nanopore DNA sequencing, massively parallel signature sequencing (MPSS), Polony sequencing, 454 pyrosequencing, combinatorial probe anchor synthesis (cPAS), Ion Torrent semiconductor sequencing, DNA nanoball sequencing, and SOLiD sequencing.

For methods that utilize a high-throughput sequencing method, the amplicons must be converted into a sequencing library for sequencing. A “sequencing library” is a pool of DNA fragments that include adapters. Thus, in these embodiments, the methods may further comprise (a) fragmenting the amplicons, and/or (b) adding adapters to the amplicons. Methods of generating sequencing libraries are well known in the art. Adapters must be included in or added to the amplicons to allow them to interact with a high-throughput sequencing platform. In some embodiments, adapters are included in the primers disclosed herein such that the adapters are added to the amplicons during the DNA amplification step. In other embodiments, adapters are ligated to the amplicons following the DNA amplification step using a ligase enzyme.

In step (d), the sequencing reads are analyzed. DNA sequencing produces sequencing reads, i.e., sequences of the DNA fragments present in the sequencing library as determined by the sequencer. To analyze the sequencing reads, they are first cleaned up (e.g., trimmed, filtered for quality, de-noised to limit the impact of sequencing errors, de-replicated to reduce file size). Without further analysis, the resulting sequences lack genomic context. Thus, to determine the source of a read (i.e., organism from which the sequenced DNA fragment was derived), it must be mapped to a reference database. Methods for mapping sequencing reads to reference databases are available in the form of free tools, including mothur, QIIME, and various R packages.

Any type of sample may be tested for the presence of eukaryotic endosymbionts using the methods of the present invention. Suitable samples include, but are not limited to, clinical samples (e.g., blood, serum, plasma, mucus, urine, feces, saliva, tissue), environmental samples (e.g., soil, plant, water), food samples (e.g., meat, dairy, produce), and the like.

### Methods for Treating Parasitic Infections:

In a sixth aspect, the present invention provides methods for diagnosing and treating a subject with a parasitic infection. The methods comprise: (a) obtaining a sample from the subject; (b) extracting DNA from the sample; (c) amplifying the DNA using a primer set described herein to generate amplicons; (d) sequencing the amplicons to generate sequencing reads; (e) analyzing the sequencing reads to detect the presence of a parasite in the sample; and (f) treating the subject for the detected parasite.

As used herein, a “parasitic infection” is an illness caused by a parasite. Symptoms of parasitic infections may include fever, fatigue, intestinal symptoms, skin rashes, or neurological symptoms. Parasites are commonly acquired by eating contaminated food or undercooked meat, drinking contaminated water, touching contaminated surfaces, and bug bites.

In these methods, the sample is a biological sample obtained from a subject. Exemplary subject samples include stool, peripheral blood, sera, plasma, ascites, urine, cerebrospinal fluid, sputum, saliva, bone marrow, synovial fluid, aqueous humor, amniotic fluid, cerumen, breast milk, bronchoalveolar lavage fluid, semen, prostatic fluid, Cowper's fluid or pre-ejaculatory fluid, female ejaculate, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mammary secretions, mucosal secretion, stool, stool water, pancreatic juice, lavage fluids from sinus cavities, bronchopulmonary aspirates, blastocoel cavity fluid, umbilical cord blood, a skin swab sample, a throat swab sample, a genital swab sample, and an anal swab sample. However, in preferred embodiments, the sample is a blood sample or fecal sample. In some embodiments, the sample is suspected of containing a parasite.

In the Examples, the inventors found that a primer set comprising the forward primer of SEQ ID NO: 3 and the reverse primer of SEQ ID NO: 6 works best for amplifying 18S gene sequences from fecal samples. Thus, in some embodiments, the sample is a fecal sample and the primer set comprises the forward primer of SEQ ID NO: 3 and the reverse primer of SEQ ID NO: 6.

The “subject” that is diagnosed and treated using these methods may be any vertebrate animal, including fish, birds, mammals, amphibians, and reptiles. Examples of suitable mammals include, but are not limited to, humans, cows, horses, sheep, pigs, goats, rabbits, dogs, cats, bats, mice, and rats. In certain embodiments, the methods may be performed on lab animals (e.g., mice, rats) for research purposes. In other embodiments, the methods are used to treat commercially important farm animals (e.g., cows, horses, pigs, rabbits, goats, sheep, chickens) or companion animals (e.g., cats, dogs). In some embodiments, the subject is suspected of having a parasitic infection. In preferred embodiments, the subject is a human.

In step (f) of the methods, the subject is treated for the detected parasite. As used herein, “treating” or “treatment” describes the management and care of a subject for the purpose of combating a parasitic infection. Treatments include methods or medications that prevent the onset of the symptoms or complications, alleviate the symptoms or complications, or eliminate the parasitic infection. Suitable treatments for parasitic infections include antiparasitic, antifungal, and antibiotic drugs. Specific examples of antiparasitic drugs include albendazole, mebendazole, metronidazole, and tinidazole. Other treatments for parasitic infections include physical interventions such as fluid aspiration, surgical removal of parasites, or resection of affected tissues. Treatment often includes prevention of re-infection using approaches such as vector control, environmental treatment, prophylactic drug administration, and behavioral modification.

### Depletion of Host DNA:

In many samples, sequencing reads of host origin crowd out reads from eukaryotic endosymbionts. Thus, in some embodiments, the methods of the present invention further comprise adding an RNA-guided nuclease and a gRNA described herein to digest host DNA (or amplicons generated therefrom) prior to sequencing. The RNA-guided nuclease and gRNA may be added (1) to DNA extracted from the sample prior to the amplification step, or (2) to the amplicons produced via the amplification step prior to the sequencing library preparation step, or (3) to the sequencing library prior to the sequencing step. In this additional step, the RNA-guided nuclease is targeted to non-parasitic 18S gene sequences via the gRNA(s), and the resulting fragments are purified away by size selection prior to sequencing. Because these gRNAs specifically target non-parasitic 18S sequences and leave eukaryotic endosymbiont 18S sequences intact, this additional step can be used to increase the efficacy of eukaryotic endosymbiont detection by depleting host DNA present in the sample. In Example 3, the inventors demonstrate that the gRNAs disclosed herein are capable of digesting 18S sequences from mammals, birds, amphibians, reptiles, and fish. Thus, this additional step may be used to deplete host DNA from any vertebrate organism. This extra step may be added to any of the methods provided herein in advance of the amplification step.

As used herein, a “RNA-guided nuclease” is a nuclease that cleaves DNA/RNA and is targeted to specific DNA/RNA sequences via a gRNA. An RNA-guided nuclease can be an endonuclease or an exonuclease and can be naturally occurring or engineered. Examples of RNA-guided nucleases include Cas9, Cpf1, Cas3, Cas8a-c, Cas10, Cas13, Cas14, Cse1, Csy1, Csn2, Cas4, Csm2, Cm5, Csf1, C2c2, CasX, CasY, Cas14, and NgAgo. The RNA-guided nuclease can be from any bacterial or archaeal species. For example, in some embodiments, the RNA-guided nuclease is from Streptococcus pyogenes, Staphylococcus aureus, Neisseria meningitidis, Streptococcus thermophiles, Treponema denticola, Francisella tularensis, Pasteurella multocida, Campylobacter jejuni, Campylobacter lari, Mycoplasma gallisepticum, Nitratifractor salsuginis, Parvibaculum lavamentivorans, Roseburia intestinalis, Neisseria cinerea, Gluconacetobacter diazotrophicus, Azospirillum, Sphaerochaeta globus, Flavobacterium columnare, Fluviicola taffensis, Bacteroides coprophilus, Mycoplasma mobile, Lactobacillus farciminis, Streptococcus pasteurianus, Lactobacillus johnsonii, Staphylococcus pseudintermedius, Filifactor alocis, Legionella pneumophila, Suterella wadsworthensis Corynebacter diphtheria, Acidaminococcus, Lachnospiraceae bacterium, or Prevotella.

### Advantages:

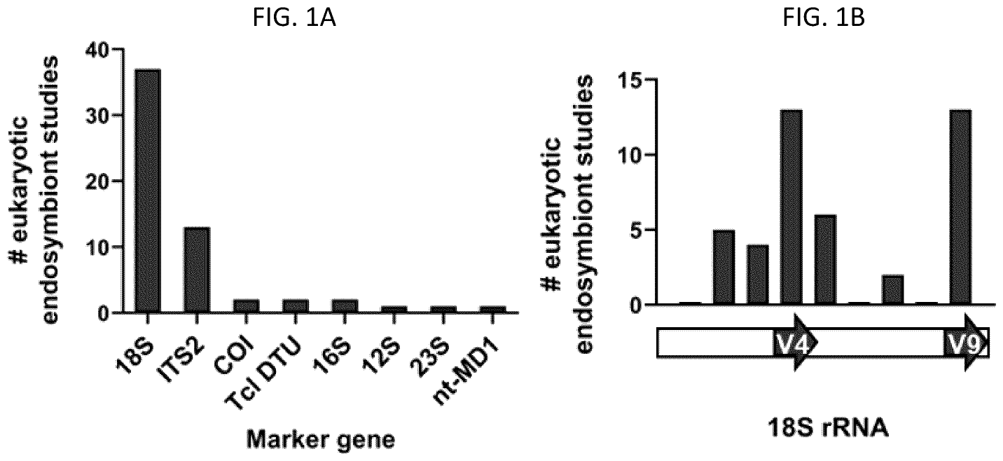

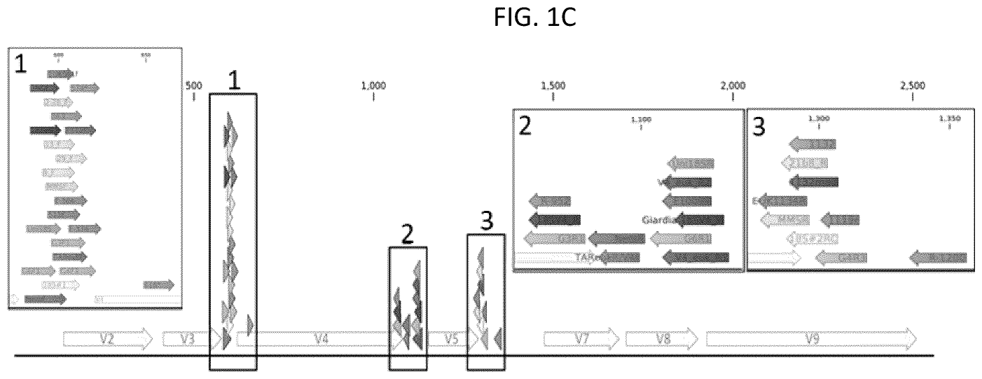

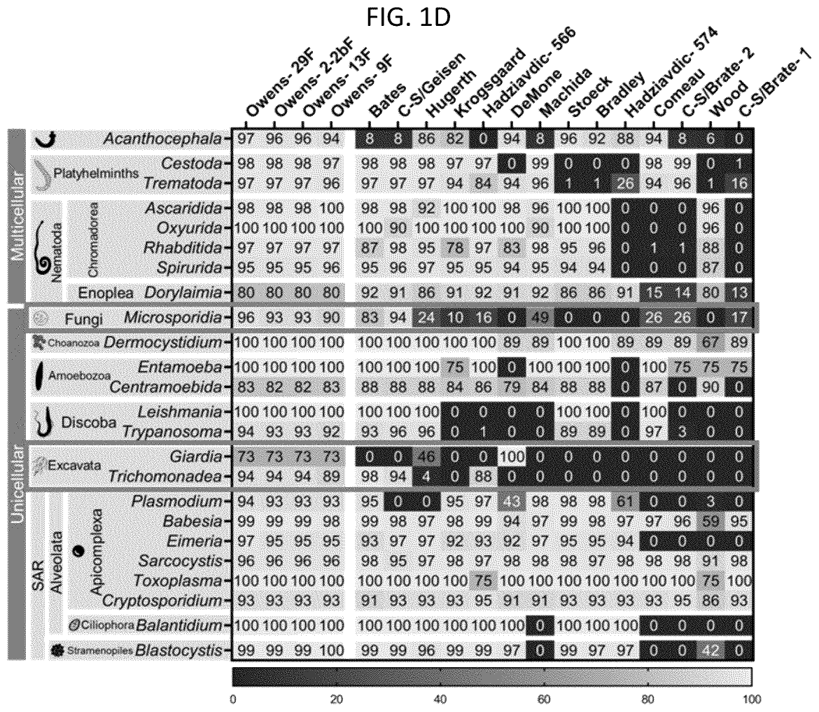

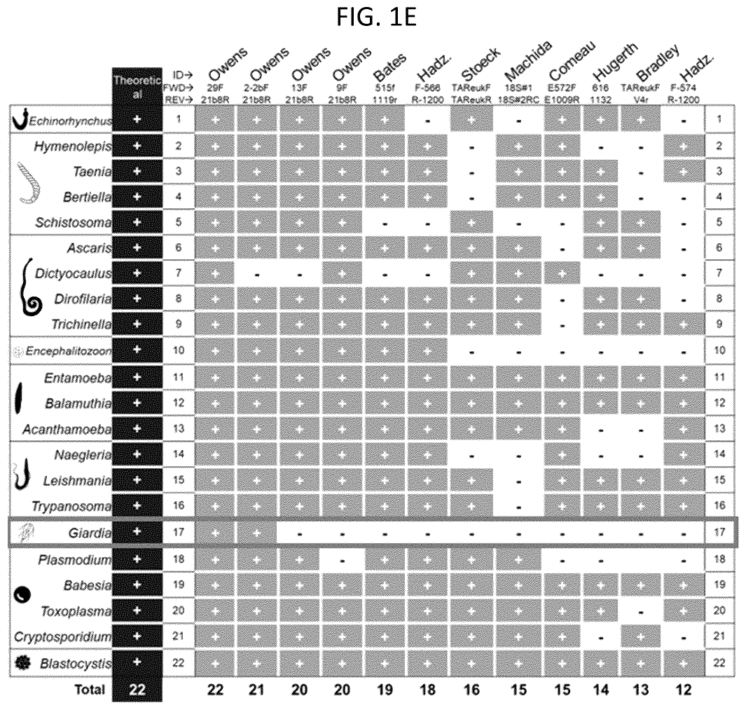

In Example 1, the inventors determined (via an in silico analysis) that their primers amplify the 18S gene from eukaryotic endosymbionts in all 24 clinically relevant parasite clades, i.e., Acanthocephala, Cestoda, Trematoda, Ascaridida, Oxyurida, Rhabditida, Spirurida, Dorylaimia, Microsporidia, Dermocystidium, Entamoeba, Centramoebida, Leishmania, Trypanosoma, Giardia, Trichomonadea, Plasmodium, Babesia, Eimeria, Sarcocystis, Toxoplasma, Cryptosporidium, Balantidium, and Blastocystis (see FIG. 1D). Thus, in some embodiments, the methods of the present invention are capable of detecting eukaryotic endosymbionts from these 24 clinically relevant clades.

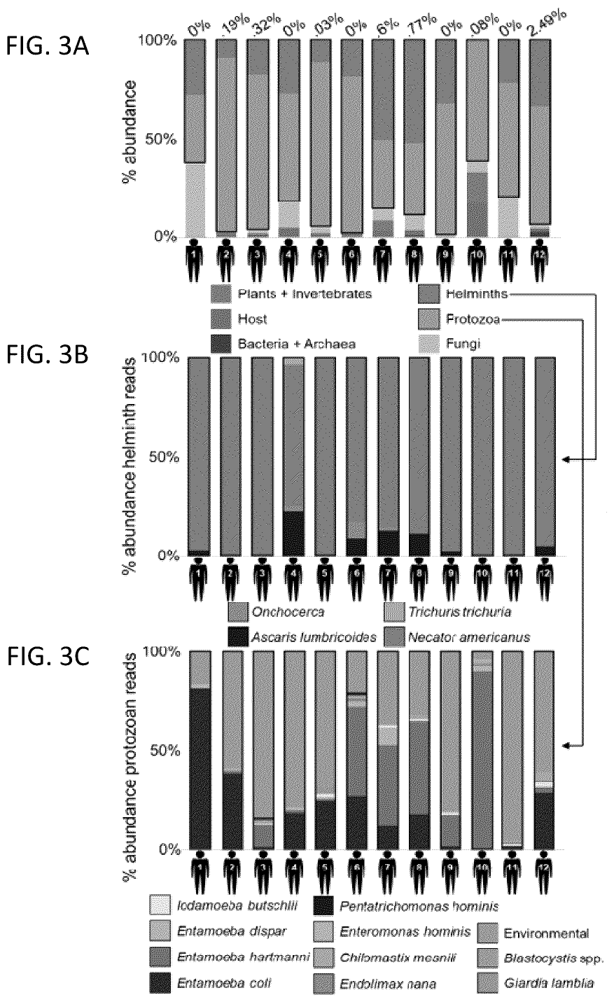

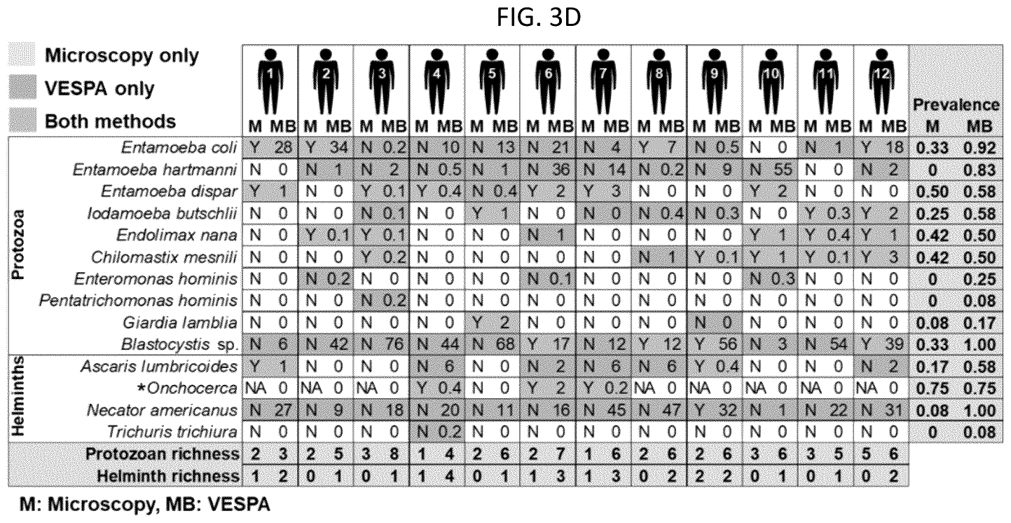

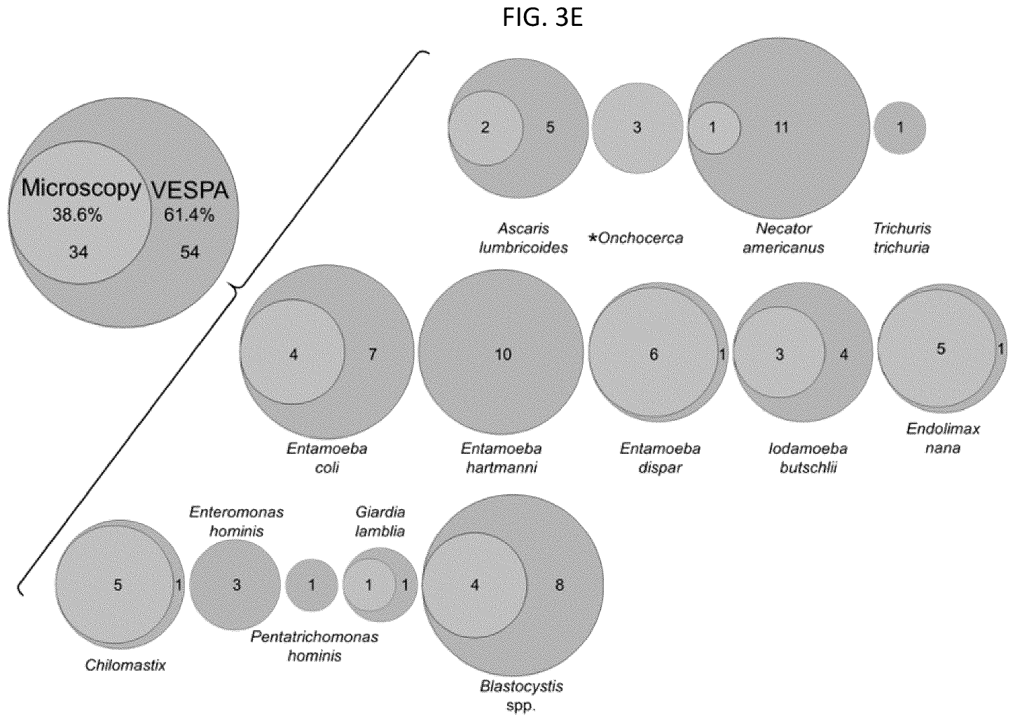

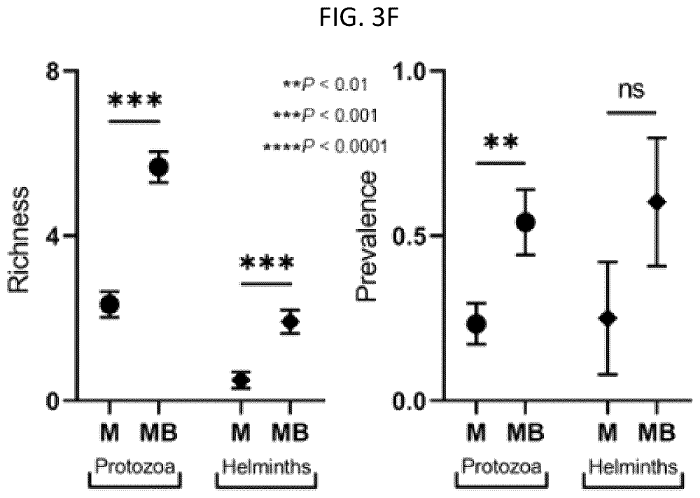

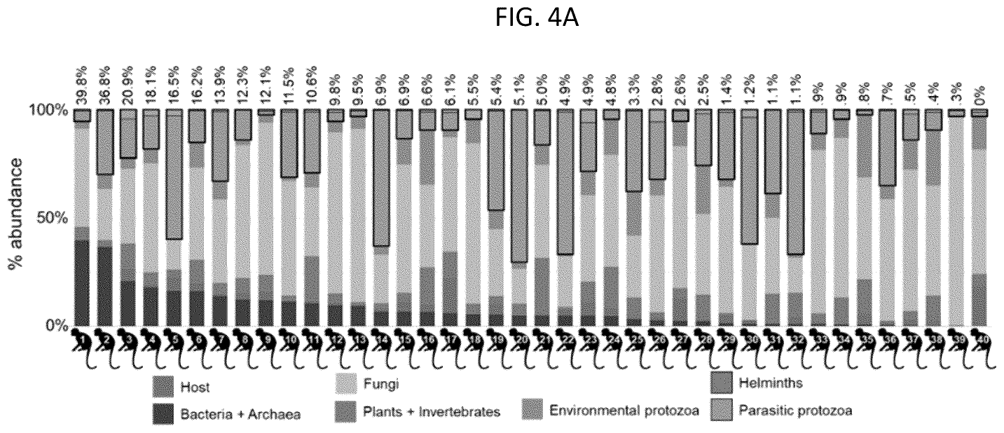

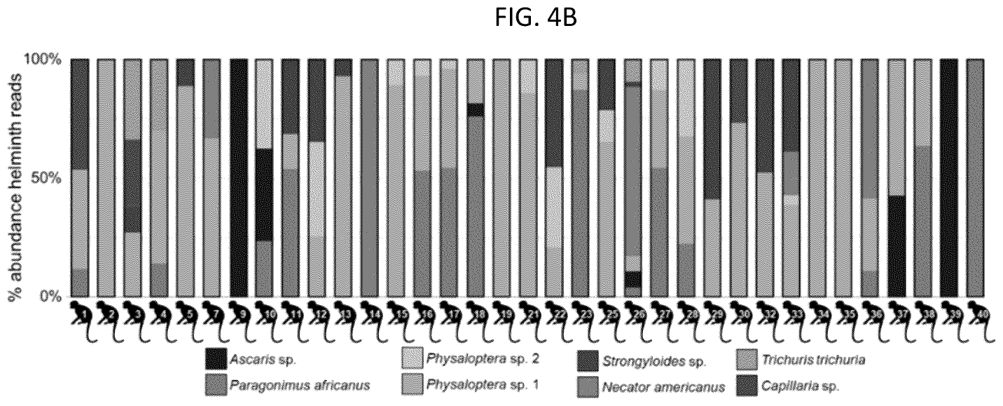

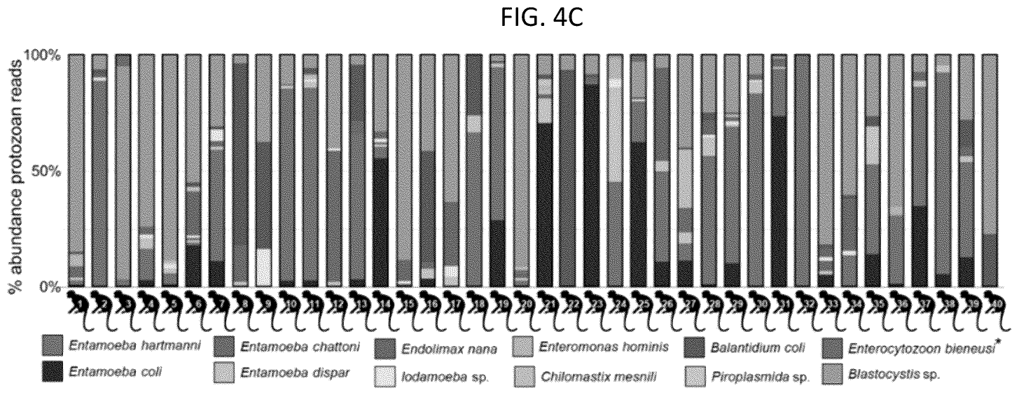

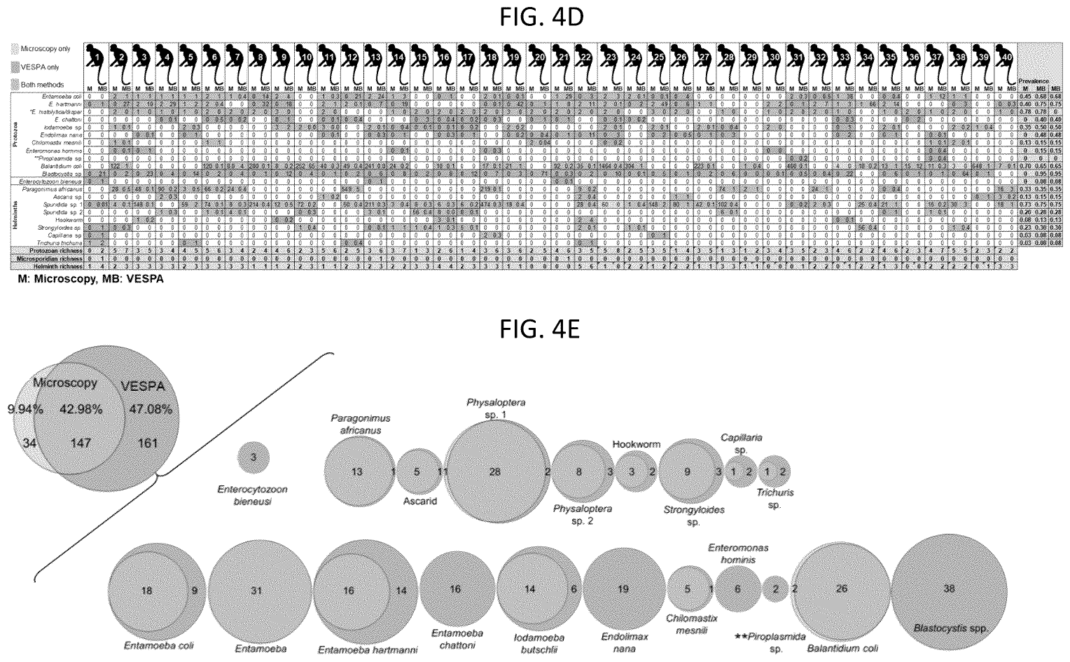

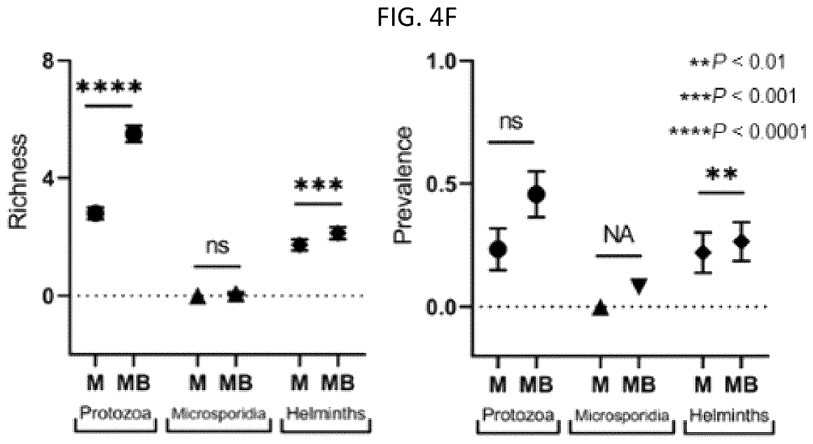

Additionally, in Example 1, the inventors demonstrate that their method produces less than 50% off-target reads (see FIG. 3A and FIG. 4A). Thus, in some embodiments, less than 50% of the sequencing reads produced by the methods of the present invention are off-target reads. As used herein, the term “off-target reads” refers to sequencing reads produced from DNA from organisms other than eukaryotic endosymbionts (e.g., bacterial DNA and vertebrate DNA).

### Kits:

In a seventh aspect, the present invention provides kits comprising one or more primer sets described herein and instructions for use. In some embodiments, the kits further comprise a gRNA described herein (i.e., to allow for host DNA depletion) and/or a mock community described herein (i.e., to allow for standardization across experiments and sample types).

Additionally, the kits may include additional reagents for use in a DNA extraction reaction, an amplification reaction (e.g., a DNA polymerase, dNTPs, amplification buffer), or a DNA sequencing reaction (e.g., adapters). As noted above, the adapters may be part of the primers or put another way the primers may include sequencing adapter or barcoding portions when supplied as part of the kit to allow for easy use of the kit and to minimize steps in the methods.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1

In the following example, the inventors describe the development of an improved sequencing-based method for detecting eukaryotic endosymbionts. They demonstrate that this method can be used to recognize all major groups of vertebrate endoparasites while amplifying relatively few off-target sequences.

### Introduction:

Microbiomes are multi-kingdom assemblages of microorganisms and their entire “theater of activity” including signaling molecules and metabolites¹. Such communities have emergent properties arising from cross-species and cross-kingdom interactions². One of the most salient examples is the human gut, wherein bacterial community dynamics have direct effects on health³ and can be manipulated to improve disease outcomes in clinical settings⁴. Evidence is mounting that assemblages of host-associated eukaryotes also form communities with important consequences for host health⁵, although they are far less studied compared to their bacterial, archaeal, and fungal counterparts⁶. Even terminology to describe host-associated eukaryotes is lacking. “Eukaryotic microbiome/microbiota”⁷ does not include host-associated macro-organisms such as helminths, “nemabiome”⁸ is limited to nematodes, and “parasites”⁹ excludes commensal/beneficial organisms and includes ectoparasites. Herein we use the term “eukaryotic endosymbionts” to refer to both microscopic eukaryotes (microsporidia, protozoa, algal parasites) and macroscopic metazoans (helminths, pentastomes). In this context, we use the prefix “endo” to include endoparasites and commensals, while excluding ectoparasites (mites, ticks, fleas). We exclude fungi because of their fundamentally different life histories¹⁰ and the fact that established methods already exist for assessing the “mycobiome”¹¹. However, we include microsporidia, because their life cycles are considered more similar to protozoa than to fungi¹².

Well-established methods exist to study eukaryotic endosymbiotic organisms. Microscopic observation has been an essential tool since van Leeuwenhoek first described Giardia in the seventeenth century¹³. Combined with subsequent advances in staining and enrichment techniques, microscopy is still a gold standard method¹⁴, although it requires specialized training¹⁵ and has inherent resolution limits (i.e., some species cannot be distinguished solely based on morphology, a phenomenon known as “cryptic species complexes” ¹⁶). For example, the genus Entamoeba contains pathogenic E. histolytica and benign E. dispar which appear identical under the microscope¹⁷. More recently developed molecular assays (e.g., PCR and DNA sequencing of amplicons) have enabled finer taxonomic differentiation, including strain-level identification of species complexes¹⁸. Although extremely useful, such assays usually have high DNA sequence specificity and are therefore not suitable for characterizing diverse assemblages of eukaryotic endosymbionts.

Methods for characterizing bacterial and fungal assemblages are standardized and based on massively parallel sequencing of amplified marker genes, or metagenomic barcoding (henceforth metabarcoding)¹⁹. For bacteria, the 16S ribosomal RNA (16S rRNA, or just 16S) locus²⁰ and for fungi, the internal transcribed spacer (ITS) locus²¹ are proven targets for metabarcoding. By contrast, “universal” targets and protocols for metabarcoding of eukaryotic endosymbionts are not standardized⁶. For example, some published methods utilize PCR primer sets originally designed for free-living eukaryotic microbes²²⁻²⁵, some target metazoans only^{26,27}, while others focus exclusively on helminthss,^{8,28-30} or gut-associated organisms³¹⁻³³. There is also a conspicuous absence of published comparisons to “gold standard” methods such as microscopy³⁴. Moreover, no commercially available reagents exist for assessing the accuracy of eukaryotic endosymbiont metabarcoding-based methods. Community standards (mixtures of organisms or their genetic material in known composition and quantity) have been important for standardizing microbiome protocols and are commercially available³⁵. Unfortunately, no such standard exists for eukaryotes other than fungi.

Here we present VESPA (Vertebrate Eukaryotic endoSymbiont and Parasite Analysis), a new methodology for eukaryotic endosymbiont metabarcoding that resolves the issues described above. We compare VESPA to published methods in silico and using a new community standard comprised of cloned DNA from eukaryotic endosymbiont lineages across the Tree of Life. We then compare our new method to the “gold standard” of microscopy using clinical samples. Our results show that VESPA and our community standard constitute a major advance that should enable “microbiome-like” insights into the structure and function of vertebrate-associated eukaryotic endosymbiont communities.

### Rationale:

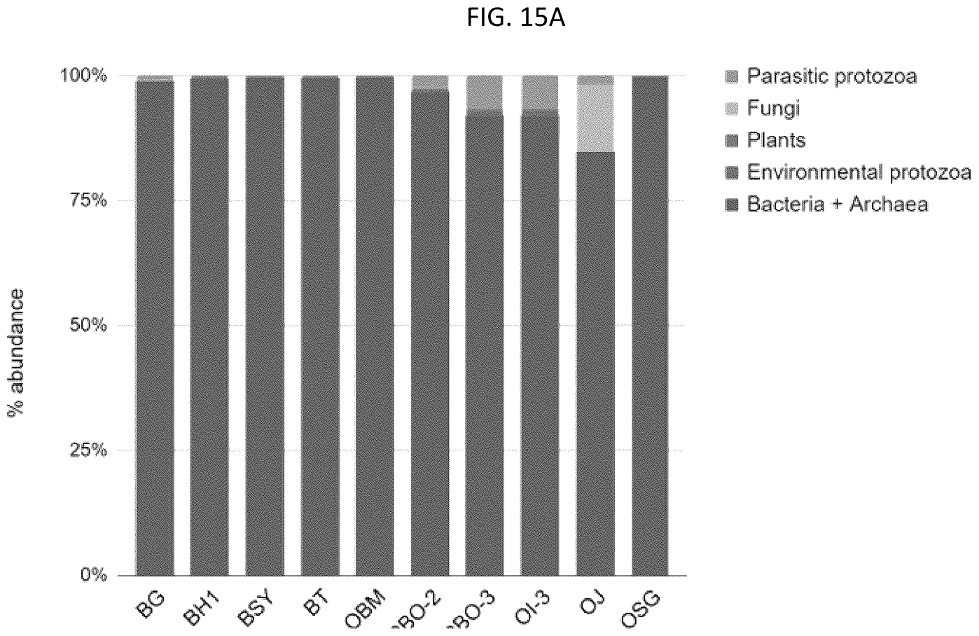

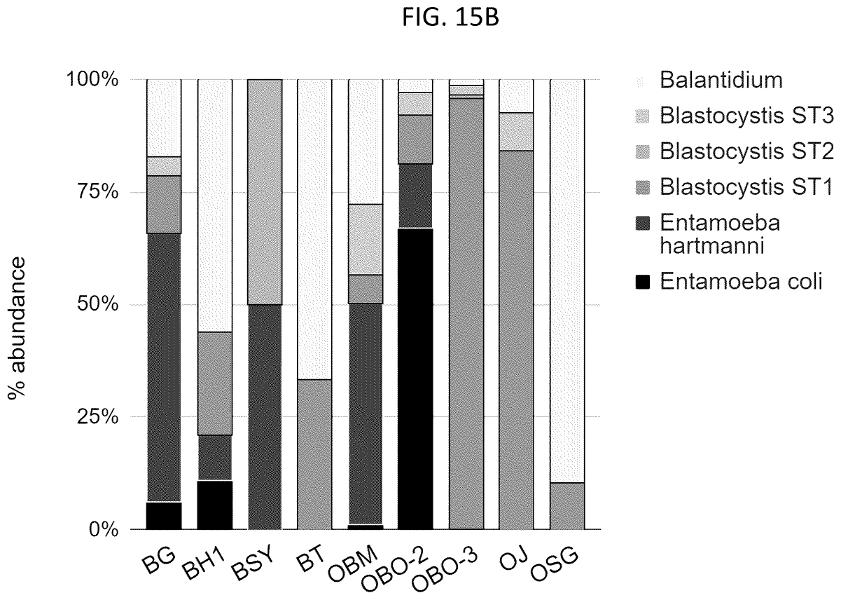

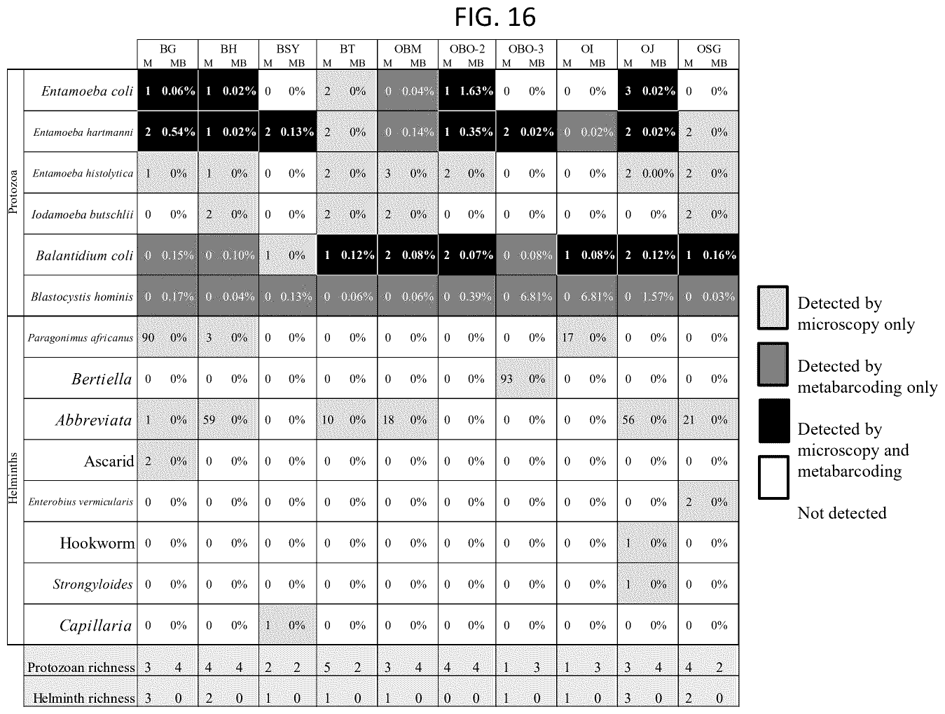

Our goal at the outset of this work was to establish a eukaryotic endosymbiont metabarcoding pipeline as an alternative to standard methods of microscopy and group-specific PCR for use in ongoing studies. Several eukaryotic endosymbiont metabarcoding studies had already been published, so we chose one such protocol and validated it in our lab. To validate the protocol, we used DNA isolated from a set of non-human primate fecal samples, which had been previously characterized by microscopy as part of a completed study (n=10). By microscopic examination, all 10 samples contained at least 1 protozoan organism (total population richness=5) and 9 of 10 contained at least one helminth (total population richness=8). We used the Earth Microbiome Project (EMP; mSystems 3(3), 2018) protocol to amplify the hypervariable 9 region (V9 hereafter) of the 18S small subunit ribosomal RNA gene (18S hereafter) and sequence the resulting amplicon libraries. Specifically, we used the forward primer 1391f (GTACACACCGCCCGTC; SEQ ID NO: 80) and the reverse primer EukBr (TGATCCTTCTGCAGGTTCACCTAC; SEQ ID NO: 81). The majority of the reads obtained using the EMP protocol (total mean reads after quality filter=33,513 per sample, range: 4,222-126,119) were identified as from bacteria or archaea (prokaryotic read mean=96.1% per sample, range: 84. 5%-99.7%; FIG. 15A). The finding of off-target 16S prokaryotic reads was not unexpected based on published results. Nonetheless, this result was not ideal because, in such high numbers, these off-target reads can introduce bias and mask the presence of rarer organisms. In total, six parasitic/commensal protozoans were identified including three subtypes of Blastocystis (FIG. 15B) and the average protozoan richness per sample was similar between the two methods (average richness by microscopy=3.0, average richness by metabarcoding=3.2; FIG. 16, top panel). Unexpectedly, for helminths, despite an average sample richness by microscopy of 1.5, no helminths were detected by metabarcoding (FIG. 16, bottom panel).

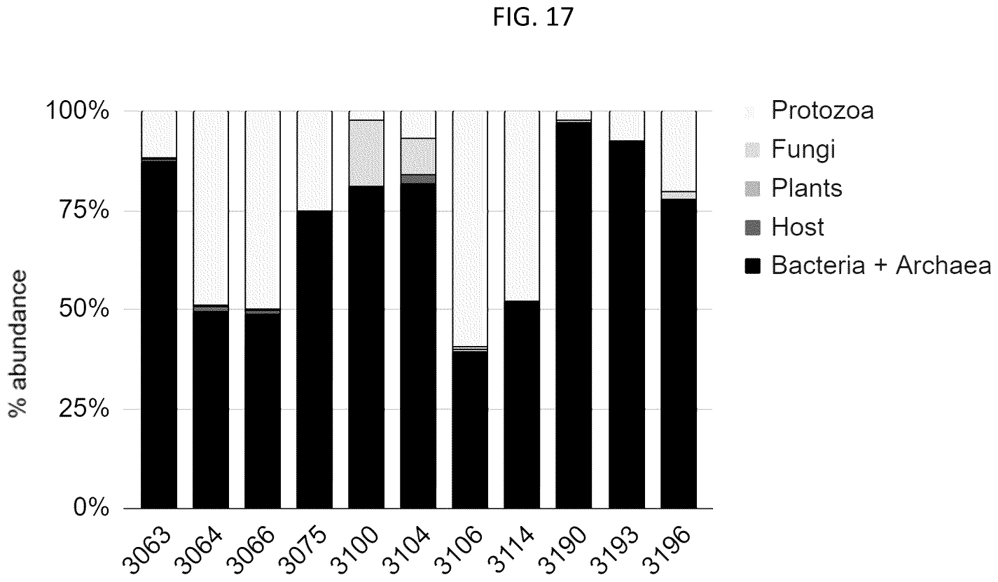

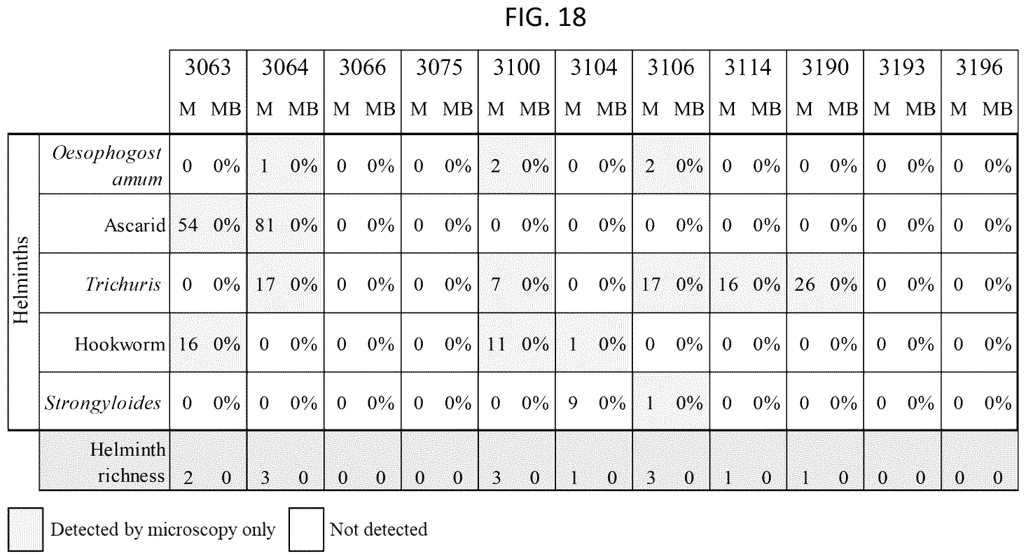

To further investigate the preponderance of prokaryotic sequences and lack of helminth coverage, we performed 18S V9 metabarcoding using the EMP protocol on a more common sample type, human fecal samples (n=11), which were previously characterized for soil-transmitted helminths (total population richness=5) as part of a concluded study. Similarly, the majority of the reads obtained (total mean reads after quality filter=11,018 per sample, range: 2,320-20,053) were bacterial or archaeal in origin (prokaryotic read mean=71.5% per sample, range: 39.3%-96.9%) and very few helminth reads were detected (FIG. 17). Several annotated sequence variants (ASVs) were identified in the QIIME2 pipeline as Rhabditiform nematodes but were later re-classified as archaeal sequences based on BLAST analysis. No correctly classified helminth reads were numerous enough to pass the quality-filtering threshold of 0.01% total reads per sample, in contrast to microscopy findings in which helminth prevalence was 0.7 and mean richness was 1.5 (range: 1-3, FIG. 18).

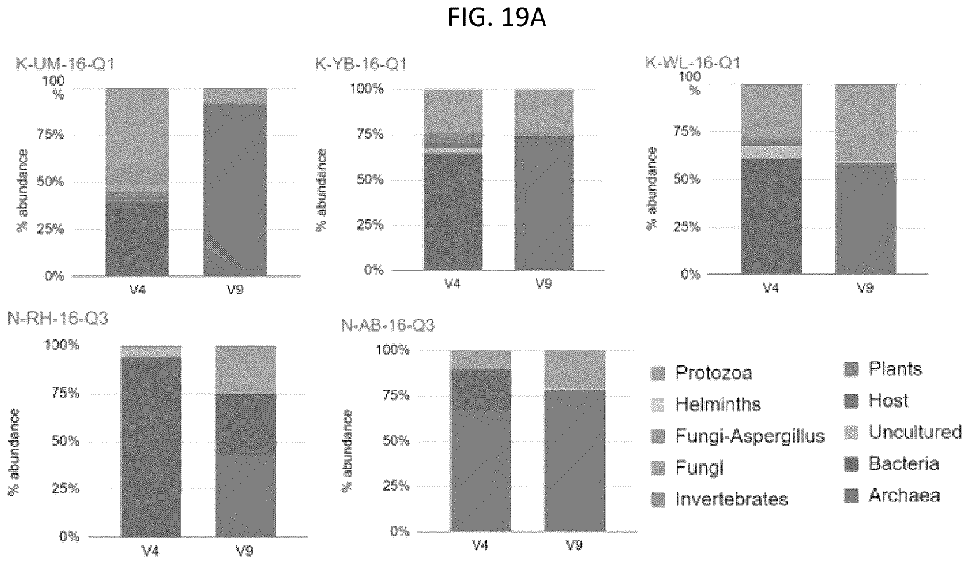

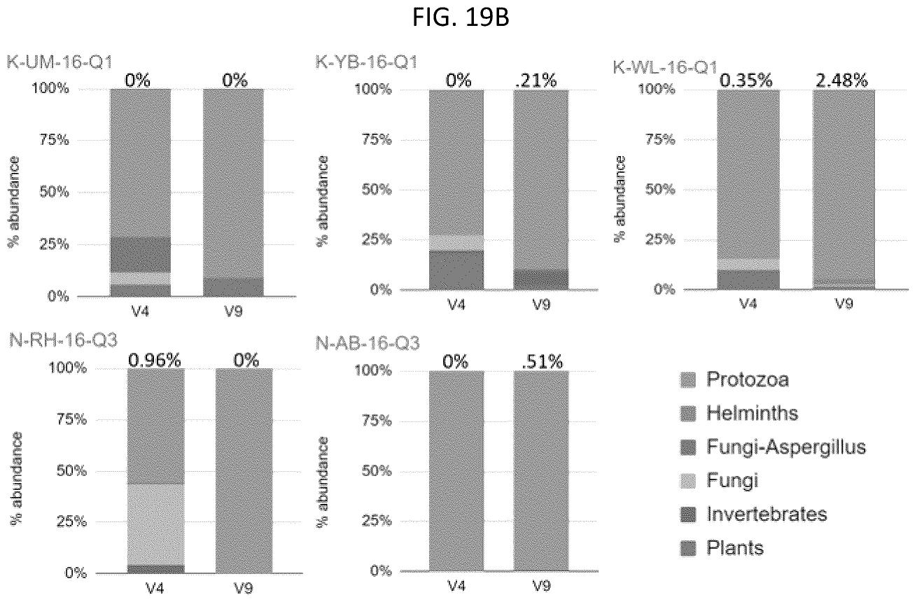

It was possible that the high number of off-target reads was masking the presence of less-abundant target reads (i.e., helminths), so we hypothesized that a different method that results in fewer prokaryote reads would yield more target identifications. We chose a set of 18S hypervariable region 4 (V4 hereafter) primers, i.e., forward primer E572F and reverse primer E1009R (see Table 5 for primer sequences) that had been used in studies on fecal samples with no reports of issues with prokaryotic reads and performed metabarcoding using this protocol alongside the EMP protocol for comparison. As starting material, we used non-human primate fecal samples (n=5) that were known to contain both protozoan and helminth parasites. Because it was possible that our previous sequencing depth was not sufficient to detect less abundant organisms, we sequenced more deeply than in previous experiments (total mean reads after quality filter=64,081 per sample, range: 8,201-109,545). Generally, V9 primer data tended to include more archaeal sequences and V4 primer data more bacterial sequences, but in both cases the majority of reads were identified as off-target amplification of prokaryotic origin (V4 prokaryotic read mean=72.0% per sample, range: 50.5%-94.2%; V9 prokaryotic read mean=75.4% per sample, range: 57.4%-91. 5%; FIG. 19A). After filtering out the reads from prokaryotes, uncultured environmental sequences, and the host organism, the remaining reads were dominated by fungal, plant, and protozoal sequences with very few helminth reads (V4 helminth read mean=0.26% per sample, range: 0%-0.96% per sample, prevalence=0.4; V9 helminth read mean=0.62% per sample, range: 0%-2.48% per sample, prevalence=0.6; FIG. 19B), despite our expectation that all samples would contain at least one helminth.

In view of the unsatisfying results generated using these published methods, we performed an extensive literature review to identify additional methods for eukaryotic endosymbiont metabarcoding and we ultimately designed our own method.

### Results:

Here we compile and evaluate published methods for metabarcoding vertebrate-associated eukaryotic endosymbionts and choose a marker gene and region for amplification. We then compare the relevant subset of published methods to a new method of our own design in a progressive series of experiments. We begin with in silico PCR, proceed to amplification of single parasite DNA templates, and then conduct metabarcoding using an engineered mock community standard. We finally apply the best-performing protocol to clinical samples from humans and non-human primates and compare results to those obtained with microscopy.

### Methods Review and New Method Design

In a literature review consisting of 54 papers that used amplicon sequencing (metabarcoding) to characterize eukaryotic assemblages in vertebrate hosts, we identified eight marker genes, including 16S (n=1), nt-MID1 (n=1), 12S (n=1), mitochondrial 16S (n=1), 28S (n=1), mini-exon Tcl DTU (n=2), CO1 (n=2), ITS-2 (n=13), and 18S (n=37; FIG. 1A). Of these publications, 25 targeted specific sub-groups (e.g., nematodes or trypanosomes) and 29 used a pan-parasite/commensal approach. Based on the widespread incorporation of small subunit ribosomal RNA 18S gene (18S hereafter) sequences into databases, the standardized use of the counterpart prokaryotic 16S gene for bacterial metabarcoding, and evidence that non-protein coding genes outperform protein-coding genes as metabarcoding markers³⁶, we chose to pursue 18S as our marker gene.

18S contains hypervariable regions V1-V9, and the regions that were most commonly targeted in the studies reviewed were V4 (n=13) and V9 (n=13; FIG. 1B). The 18S V4 region has the highest entropy within the size limits of MiSeq v2 chemistry³⁷ and therefore the highest taxonomic resolution for this commonly used metabarcoding platform, so we chose to target this region. We identified a total of 22 published sets of V4 primers. Additionally, we created new 18S V4 primers designed to target all eukaryotic endosymbionts, consisting of 4 candidate forward primers and one reverse primer (see methods section for details on primer design, Table 5 for primer sequences, and FIG. 1C for a map of primer binding sites).

Testing metabarcoding methods for taxonomic coverage using in silico PCR Testing all 22 published 18S V4 primer sets in silico yielded an average eukaryotic endosymbiont coverage of 64.9% (Table 1, bolded columns). No primer set recognized both Plasmodium and Giardia, and 9 of 19 did not recognize either (Table 1, final two columns). We found significant off-target coverage (>5%) of bacterial and/or archaeal groups for 4 of 22 sets (Table 1, asterisks), and the primer set with the highest overall eukaryotic coverage (96.3%; Hugerth 2014 “563/1132”) also had the highest coverage of archaea and bacteria (47.9% and 72.0% respectively; Table 1). Primer sets with >5% off-target coverage were not analyzed further.

In silico PCR including our 4 new primer sets alongside the remaining 18 published 18S V4 sets yielded coverage data spanning a wide range (5.8% to 98.0%; Table 2). Across target groups (normalizing by eligible accessions), our newly designed primers had the highest mean percent coverage, at 95.2%-96.8%, and the best complementarity as evidenced by the lowest score in a rank sum analysis (Table 2, final column).

TABLE 1
In silico taxonomic coverage for published 18S V4 primer sets

| | | | | Eukaryotic | | | |
|---|---|---|---|---|---|---|---|
| | | Off-target | | endosymbiont | | Specific | |
| | | groups | | groups | | examples | |
| | | n= | | | | | |
| | | 20,197 | 381,535 | 4,229 | 15,265 | 198 | 23 |
| Reference ID | Primers | Archaea | Bacteria | Helminths | Protozoa | Plasmodium | Giardia |
| Bates 2012 | 515f/1119r | 0 | 0 | 80.4 | 95.9 | 94.8 | 0 |
| Bower 2004* | 18SEUK581F/1134R | 46.2* | 8.2* | 0.4 | 82.4 | 0 | 72.7 |
| Bradley 2016 | TAReuk454F1/V4r | 0 | 0 | 48.9 | 67.1 | 97.9 | 0 |
| Cavalier-Smith 2009, Brate 2010 | 3NDf/V4_euk_R2 | 0 | 0 | 50.8 | 22.8 | 0 | 0 |
| Cavalier-Smith 2009, Brate 2010 | 3NDf/V4_euk_R1 | 0 | 0 | 5.8 | 21.1 | 0 | 0 |
| Cavalier-Smith 2009, Giesen 2010 | 3NDf/1132mod | 0.3 | 0 | 80.7 | 94.2 | 0 | 0 |
| Comeau 2011 | E572F/E1009R | 0 | 0 | 65.3 | 44.5 | 0 | 0 |
| DeMone 2020** | 18SV4_F/-R | 0 | 0 | 86.4 | 62.3 | 42.8 | 0 |
| Hadziavdic 2014 | F-566/R-1200 | 0 | 0 | 76.4 | 81 | 99.6 | 0 |
| Hadziavdic 2014 | F-574/R-952 | 0 | 0 | 48.3 | 62.9 | 61.3 | 0 |
| Hugerth 2014* | 574/1132 | 12.5* | 0 | 80 | 94.2 | 0 | 0 |
| Hugerth 2014 | 616/1132 | 3.3 | 0.2 | 93.1 | 75.8 | 0 | 45.5 |
| Hugerth 2014* | 563/1132 | 47.9* | 72* | 96.1 | 96.4 | 0 | 100 |
| Krogsgaard 2018** | G31/G43/G61 | 0 | 0 | 78.5 | 67 | 94.8 | 0 |
| Machida 2012 | 18S#1/#2RC | 0 | 0 | 78.1 | 45.2 | 97.9 | 0 |
| Sikder 2020* | MMSF/R | 17.5* | 0 | 79.3 | 42.7 | 0 | 0 |
| Stoeck 2010 | TAReuk454F1/R3 | 0 | 0 | 49.1 | 78.4 | 97.9 | 0 |
| Wood 2013 | Nem18SFlong/R | 0 | 0 | 32.2 | 25.2 | 2.6 | 0 |
| Zhan 2013 | Uni18S/R | 0 | 0 | 72.8 | 64 | 0 | 100 |

Numbers shown are % coverage allowing for 1 mismatch with a 2-base pair 3′ window using the SILVA 138.1 SSU rRNA NR Ref database;
n, number of total eligible accessions;
*removed from further analysis due to high prokaryotic complementarity;
**multiple primer sets were combined for analysis.

TABLE 2
In silico taxonomic coverage of helminths and protozoa
for published and newly designed 18S V4 primer sets

| | | | n = 3,097 | n = 2,913 | Rank | | Rank |
|---|---|---|---|---|---|---|---|
| Short ID | Primers | Mean | Helminths | Protozoa | Helminths | Protozoa | sum |
| Owens- 29F | 29F/21b8R | 96.8% | 95.5% | 98.0% | 1 | 1 | 2 |
| Owens- 2-2b | 2-2F/21b8R | 96.4% | 94.9% | 97.9% | 2 | 2 | 4 |
| Owens- 13F | 13F/21b8R | 96.4% | 94.9% | 97.9% | 2 | 2 | 4 |
| Owens- 9F | 9F/21b8R | 95.2% | 94.4% | 96.0% | 4 | 4 | 8 |
| Bates | 515f/1119r | 88.2% | 80.4% | 95.9% | 8 | 5 | 13 |
| Hugerth | 616/1132 | 84.5% | 93.1% | 75.8% | 5 | 8 | 13 |
| Krogsgaard** | G3/G4/G6 | 81.0% | 93.0% | 69.0% | 6 | 9 | 15 |
| Hadziavdic- 566 | F-566-R-1200 | 78.7% | 76.4% | 81.0% | 10 | 6 | 16 |
| DeMone** | 18SV4F/R/GR | 75.3% | 86.4% | 64.1% | 7 | 11 | 18 |
| Stoeck | TAReukF1/R3 | 63.8% | 49.1% | 78.4% | 13 | 7 | 20 |
| Machida | 18S#1/18S#2 | 61.7% | 78.1% | 45.2% | 9 | 14 | 23 |
| Bradley | TAReukF1/V4r | 58.0% | 48.9% | 67.1% | 14 | 10 | 24 |
| Hadziavdic- 574 | F-574/R-952 | 55.6% | 48.3% | 62.9% | 15 | 12 | 27 |
| Comeau | E572F/E1009R | 54.9% | 65.3% | 44.5% | 11 | 15 | 26 |
| C-S/Giesen* | 3NDf/1132 | 47.5% | 40.7% | 54.2% | 16 | 13 | 29 |
| C-S/Brate- 2* | 3NDf/V4eukR2 | 36.8% | 50.8% | 22.8% | 12 | 17 | 29 |
| Wood* | Nem18SFl/R1 | 28.7% | 32.2% | 25.2% | 17 | 16 | 33 |
| Zhan* | Uni18S/UniR | 14.0% | 5.7% | 22.3% | 19 | 18 | 37 |
| C-S/Brate- 1* | 3NDf/V4eukR1 | 13.5% | 5.8% | 21.1% | 18 | 19 | 37 |

Shaded rows, primers designed in this study;
%, % coverage calculated allowing for 1 mismatch with a 2-base pair 3′ window using the SILVA 138 SSU rRNA NR Ref database;
n, number of eligible accessions; Mean, mean coverage of all parasite/commensal groups;
*<50% overall mean target complementarity;
**multiple primer sets combined for analysis.

In silico coverage analysis using finer-resolution groups (FIG. 1D) showed that our new primers consistently amplified (defined as coverage of 50% or higher) all 24 clades of eukaryotes tested whereas no other primer sets did. Particularly problematic were Giardia (recognized by our primers and one other set in which a second reverse primer must be used to specifically amplify Giardia), Microsporidia (recognized by our primers and two other sets), and Trichomonadea (recognized by our primers and three other sets; FIG. 1D, red boxes). All methods that amplified an overall mean <50% of target sequences (n=5; Table 2, asterisks) or that required >1 primer set (n=2; Table 2, double asterisks) were not analyzed further.

### Testing Metabarcoding Methods for On-Target Amplification Using Purified DNA

In PCR amplification of genomic DNA (gDNA) from 22 individual eukaryotic endosymbiont organisms (Table 6), all four sets of candidate primers amplified more organisms than did any of the published primer sets (Owens 29F: 22 of 22, Owens 2-2bF: 21 of 22, Owens 13F: 20 of 22, Owens 9F: 20 of 22), followed by the Bates (19 of 22), Hadziavdic (18 of 22), and Stoeck (16 of 22) sets (FIG. 1E). Furthermore, two of the new sets were the only primers to successfully amplify 18S V4 from Giardia gDNA (Owens 29F and Owens 2-2bF), as expected based on in silico data (FIG. 1E, row 17).

### Testing Metabarcoding Methods for Amplification Bias Using a Community Standard

Community standards are not available for eukaryotic endosymbionts, so we collected protozoa (n=10), helminths (n=5), and a microsporidian (n=1) (Table 7) from various sources (e.g., specimen repositories, veterinary post-mortem examinations). We then isolated 18S genes from these samples and mixed them at an equimolar ratio to create a community standard, which we named “EukMix” (FIG. 2A). Metabarcoding EukMix as input with previously published and newly designed primers allowed us to directly compare empirical read abundances for each organism to their predicted (equal) abundances (FIG. 2B). The abundances of six organisms were underestimated by every primer set, and the abundances of three organisms were overestimated by every primer set (Table 3), but the absolute mean difference from theoretically equal abundance was lowest with newly designed primer set Owens 29F (FIG. 2C), which also yielded abundance data statistically significantly closer to actual input levels than any other set tested (FIG. 2D). Primer set Owens 29F consistently reconstructed the EukMix community most accurately (i.e., evenly), as determined by standard diversity and evenness measures (Pielou's species evenness, Simpson's diversity index, and Shannon diversity; FIG. 2E).

TABLE 3
Equimolar EukMix metabarcoding accuracy metrics

| | | Mean distance from the theoretical by primer set | | | | | Estimated |
|---|---|---|---|---|---|---|---|
| | | Owens | Owens | Stoeck | Hadziavdic | Bates | abundance |
| | | 29F | 2-2bF | TAREuk | F-566 | 515f | pattern |
| 1 | Echinorhynchus salmonis | −1.12% | −1.36% | 1.71% | 1.48% | 4.77% | mixed |
| 2 | Hymenolepis diminuta | 4.09% | 4.75% | 0.39% | 5.51% | 5.88% | over |
| 3 | Ascaris suum | 2.48% | 7.13% | 9.48% | 8.08% | 11.28% | over |
| 4 | Dirofilaria immitis | 0.66% | −1.91% | 2.14% | 4.39% | 0.15% | mixed |
| 5 | Trichinella spiralis | −0.57% | −3.40% | −6.22% | −5.91% | −5.84% | under |
| 6 | Encephalitozoon cuniculi | −0.14% | −0.92% | 3.56% | 1.72% | 4.12% | mixed |
| 7 | Entamoeba histolytica | −1.75% | −1.34% | −4.67% | −1.56% | −2.86% | under |
| 8 | Balamuthia mandrillaris | 0.00% | 4.11% | 11.05% | 3.26% | −1.18% | mixed |
| 9 | Naegleria fowleri | 1.03% | 2.03% | 0.98% | −5.41% | −3.86% | mixed |
| 10 | Leishmania major | −1.41% | −1.84% | −6.25% | −6.15% | −4.72% | under |
| 11 | Giardia intestinalis | −2.69% | −3.09% | −5.58% | −1.43% | −2.57% | under |
| 12 | Plasmodium falciparum | −1.02% | −5.32% | −4.84% | −3.39% | −6.18% | under |
| 13 | Babesia sp. strain MO1 | −0.98% | −4.12% | −6.22% | −5.78% | −6.04% | under |
| 14 | Toxoplasma gondii | −0.80% | −4.93% | 1.27% | −1.73% | 0.18% | mixed |
| 15 | Cryptosporidium hominis | 1.48% | 4.23% | 4.72% | 10.62% | 6.40% | over |
| 16 | Blastocystis hominis | 0.73% | 2.59% | −1.53% | −3.71% | 0.45% | mixed |

### VESPA Compared to Microscopy

Human VESPA analysis of 12 human clinical samples yielded high-quality data (Table 4) including low proportions of off-target prokaryotic reads (FIG. 3A) and host reads (host read mean=2.97 per sample, range: 0.11%-17.4%) and correspondingly high proportions of endosymbiont reads (FIG. 3B, FIG. 3C).

TABLE 4
VESPA MiSeq run metrics

| | | | Reads post- | % lost |
|---|---|---|---|---|
| Library ID | Sample type | Raw reads | quality filter | in filter |
| Human01 | Human fecal | 62,512 | 56,602 | 9.45% |
| Human02 | Human fecal | 32,755 | 30,195 | 7.82% |
| Human03 | Human fecal | 223,911 | 206,999 | 7.55% |
| Human04 | Human fecal | 43,371 | 39,228 | 9.55% |
| Human05 | Human fecal | 116,016 | 106,130 | 8.52% |
| Human06 | Human fecal | 24,095 | 22,204 | 7.85% |
| Human07 | Human fecal | 55,882 | 50,772 | 9.14% |
| Human08 | Human fecal | 80,184 | 72,324 | 9.80% |
| Human09 | Human fecal | 35,824 | 32,808 | 8.42% |
| Human10 | Human fecal | 30,176 | 27,645 | 8.39% |
| Human11 | Human fecal | 78,021 | 72,165 | 7.51% |
| Human12 | Human fecal | 123,564 | 112,774 | 8.73% |
| NHP1 | Nonhuman primate fecal | 37,377 | 35,637 | 4.65% |
| NHP2 | Nonhuman primate fecal | 98,953 | 92,910 | 6.11% |
| NHP3 | Nonhuman primate fecal | 287,932 | 269,181 | 6.51% |
| NHP4 | Nonhuman primate fecal | 56,002 | 52,080 | 7.00% |
| NHP5 | Nonhuman primate fecal | 28,351 | 26,874 | 5.21% |
| NHP6 | Nonhuman primate fecal | 104,900 | 97,907 | 6.67% |
| NHP7 | Nonhuman primate fecal | 28,409 | 26,415 | 7.02% |
| NHP8 | Nonhuman primate fecal | 25,764 | 23,788 | 7.67% |
| NHP9 | Nonhuman primate fecal | 29,434 | 27,018 | 8.21% |
| NHP10 | Nonhuman primate fecal | 58,005 | 53,206 | 8.27% |
| NHP11 | Nonhuman primate fecal | 44,422 | 39,862 | 10.26% |
| NHP12 | Nonhuman primate fecal | 36,887 | 33,991 | 7.85% |
| NHP13 | Nonhuman primate fecal | 55,101 | 49,958 | 9.33% |
| NHP14 | Nonhuman primate fecal | 34,701 | 31,934 | 7.97% |
| NHP15 | Nonhuman primate fecal | 64,954 | 60,237 | 7.26% |
| NHP16 | Nonhuman primate fecal | 50,839 | 47,371 | 6.82% |
| NHP17 | Nonhuman primate fecal | 75,005 | 68,826 | 8.24% |
| NHP18 | Nonhuman primate fecal | 76,770 | 70,964 | 7.56% |
| NHP19 | Nonhuman primate fecal | 46,543 | 44,239 | 4.95% |
| NHP20 | Nonhuman primate fecal | 40,031 | 37,507 | 6.31% |
| NHP21 | Nonhuman primate fecal | 39,344 | 36,571 | 7.05% |
| NHP22 | Nonhuman primate fecal | 29,797 | 27,118 | 8.99% |
| NHP23 | Nonhuman primate fecal | 36,615 | 33,891 | 7.44% |
| NHP24 | Nonhuman primate fecal | 84,056 | 76,577 | 8.90% |
| NHP25 | Nonhuman primate fecal | 27,672 | 26,198 | 5.33% |
| NHP26 | Nonhuman primate fecal | 32,150 | 28,996 | 9.81% |
| NHP27 | Nonhuman primate fecal | 157,483 | 144,045 | 8.53% |
| NHP28 | Nonhuman primate fecal | 31,830 | 29,320 | 7.88% |
| NHP29 | Nonhuman primate fecal | 41,127 | 37,816 | 8.05% |
| NHP30 | Nonhuman primate fecal | 60,491 | 55,710 | 7.90% |
| NHP31 | Nonhuman primate fecal | 74,435 | 67,968 | 8.69% |
| NHP32 | Nonhuman primate fecal | 59,136 | 54,146 | 8.44% |
| NHP33 | Nonhuman primate fecal | 35,473 | 32,346 | 8.82% |
| NHP34 | Nonhuman primate fecal | 39,545 | 36,508 | 7.68% |
| NHP35 | Nonhuman primate fecal | 33,505 | 31,048 | 7.33% |
| NHP36 | Nonhuman primate fecal | 44,082 | 41,003 | 6.98% |
| NHP37 | Nonhuman primate fecal | 59,451 | 54,867 | 7.71% |
| NHP38 | Nonhuman primate fecal | 14,879 | 13,872 | 6.77% |
| NHP39 | Nonhuman primate fecal | 71,275 | 64,595 | 9.37% |
| NHP40 | Nonhuman primate fecal | 62,042 | 57,199 | 7.81% |

VESPA successfully identified all three helminth and seven protozoan taxa identified with microscopy (FIG. 3D) and found these taxa in more individuals than did microscopy, with 61.4% of positive samples identified solely by VESPA (FIG. 3E). Conversely, no positives were identified by microscopy alone. Four additional taxa were found exclusively by VESPA, including one helminth, Trichuris trichiura (1 positive of 12 samples), and three protozoa, Entamoeba hartmanni (10 positives of 12 samples), Enteromonas hominis (3 positives of 12 samples), and Pentatrichomonas hominis (1 positive of 12 samples). Three of 12 patients were known by taxon-specific PCR to be infected with Onchocerca, which is not visible microscopically in feces, and all 3 were positive by VESPA. Overall, taxonomic richness was statistically significantly higher by VESPA than by microscopy for both helminths (mean richness=0.5 by microscopy, 1.92 by VESPA, Wilcoxon matched-pairs signed rank test, 2-tailed, P=0.001) and protozoa (mean richness=2.33 by microscopy, 5.67 by VESPA, Wilcoxon matched-pairs signed rank test, 2-tailed, P=0.0005; FIG. 3F, left panel). Prevalence was also higher by VESPA for helminths (mean prevalence=0.25 by microscopy, 0.60 by VESPA, Wilcoxon matched-pairs signed rank test, 2-tailed, P=0.25) and protozoa (mean prevalence=0.23 by microscopy, 0.54 by VESPA, Wilcoxon matched-pairs signed rank test, 2-tailed, P=0.002; FIG. 3F, right panel).

Non-human primate VESPA analysis of 40 non-human primate clinical samples yielded high-quality sequencing reads (Table 4) with low proportions of off-target prokaryotic reads (FIG. 4A) and host sequence reads (host read mean=3.2% per sample, range: 0%-18.49%) and correspondingly high proportions of endosymbiont reads (FIG. 4B, FIG. 4C).

VESPA successfully identified all eight helminth and six protozoan taxa identified with microscopy (FIG. 4D) and found these taxa in more individuals than did microscopy, with 47.08% of positive samples identified by VESPA only (FIG. 4E). One positive out of 29 total for a helminth (Physaloptera sp. 1) and 2 positives out of 28 total for a protozoan (Balantidium coh) were identified by microscopy only. Six additional taxa were found exclusively by VESPA: Entamoeba chattoni (16 positives of 40 samples), Endolimax nana (19 positives of 40 samples), Enteromonas sp. (6 positives of 40 samples), Piroplasmida sp. (2 positives of 40 samples), Blastocystis sp. (38 positives of 40 samples), and Enterocytozoon bieneusi (3 positives of 40 samples; FIG. 4D, FIG. 4E). Piroplasmida are intraerythrocytic parasites not visible in fecal samples and were found in 2 of 40 samples with VESPA. Thirty-one samples were positive for the Entamoeba histolytica dispar species complex by microscopy and the same 31 samples were found to be positive by VESPA but could be further taxonomically resolved as Entamoeba dispar in all cases. Richness was higher by VESPA than by microscopy for helminths (mean richness=1.73 by microscopy, 2.13 by VESPA, Wilcoxon matched-pairs signed rank test, 2-tailed, P=0.0009), protozoa (mean richness=2.8 by microscopy, 5.5 by VESPA, Wilcoxon matched-pairs signed rank test, 2-tailed, P<0.0001), and microsporidia (mean richness=0 by microscopy, 0.08 by VESPA, Wilcoxon matched-pairs signed rank test, 2-tailed, P=0.25; FIG. 4F, left panel). Prevalence was also higher by VESPA than by microscopy for all three parasite groups (helminth mean prevalence=0.22 by microscopy, 0.26 by VESPA, Wilcoxon matched-pairs signed rank test, 2-tailed, P=0.33; protozoa mean prevalence=0.22 by microscopy, 0.43 by VESPA, Wilcoxon matched-pairs signed rank test, 2-tailed, P=0.002; microsporidia mean prevalence=0 by microscopy, 0.8 by VESPA, Wilcoxon matched-pairs signed rank test, 2-tailed, P=0.25; FIG. 4F, right panel).

### Discussion:

To identify a single method for the “universal” identification of vertebrate-associated eukaryotic endosymbionts in community assemblages, we analyzed published approaches and found a wide range of amplification targets and protocols. From this literature review, we chose to focus on the 18S V4 locus and designed new primers to recognize all known groups of eukaryotic endosymbionts. We then tested published primers and our newly designed primers in a series of experiments in silico and in vitro to determine which protocols, if any, could accurately reconstruct eukaryotic endosymbiont communities. Our results clearly show that metabarcoding using newly designed primer set 29F recognizes the greatest range of endosymbionts of interest with the least off-target amplification and PCR bias of any method tested. We named our new method VESPA (Vertebrate Eukaryotic endoSymbiont and Parasite Analysis).

VESPA recognized more eukaryotic endosymbiont groups in silico than did other published methods tested, including methods that used multiple primer sets to increase coverage. Multiple primer sets, usually involving multiple independent PCR amplifications, are a feasible strategy for increasing coverage^{32,33}. However, this approach adds reagent costs and presents technical challenges related to sequencing and bioinformatics^{38,39}. Our single primer set approach should therefore reduce barriers to entry for adopting our new method. We then corroborated these in silico results with amplification of purified targets and similarly found that our primer sets amplified the greatest range of single organisms in vitro.

To examine the performance of published methods and VESPA, we directly compared assays by using an equimolar community standard, EukMix, as input for metabarcoding. Results from VESPA reflected the underlying composition of the community standard more accurately than did results from other assays. The EukMix community standard should be useful for quality control in laboratories choosing to adopt our method, and for standardization and validation, much as community standards containing bacteria and fungi have enabled standardization of microbiome protocols^{35,40}. We note that the relationship between sequencing reads and organism abundance or biomass is complicated by wide variation in 18S copy number among eukaryotic endosymbionts^{34,41}. Copy number corrections have been applied in studies of other systems^{42,43}, and such corrections could prove useful for investigations where quantifying organism abundance or biomass are the desired outputs.

Compared to microscopic examination, VESPA detected protozoa, microsporidia, and helminths in more individuals, identified additional organisms, resolved a cryptic species complex, and identified organisms not visible in fecal samples. We suspect that the greater sensitivity of VESPA results from the nature of molecular amplification—namely, that PCR can detect a theoretical minimum of one molecule of target DNA⁴⁴. Microscopy-negative samples that were PCR positive by VESPA may not have contained intact organisms or their eggs or may even have been positive by virtue of the presence of small amounts of cell-free DNA⁴⁵. In this light, we caution that our method will likely be most useful for applications where the presence of eukaryotic endosymbiont DNA is itself taxonomically informative, regardless of whether that DNA represents an intact or viable organism.

Because of the labor-intensive nature of microscopy and its dependence on trained experts, VESPA will also be useful for studies which are large-scale or performed in multiple laboratories, where labor costs and inter-observer variability would otherwise be impractical. In this light, we note that microscopy identified three positive samples not identified by VESPA in non-human primates. We suspect that these findings may represent microscopy false positives, especially because these two taxa (Physaloptera and Balantidium) are notoriously difficult to identify morphologically^{46,47}.

Our contribution with this work is a publicly available protocol for metabarcoding eukaryotic endosymbiont communities that outperforms published methods by every measure examined. VESPA is intentionally designed to have broad applicability, from microbial ecology to parasitology to clinical diagnostics. Although we tested VESPA using Illumina sequencing technology, it should be readily adaptable to other amplicon sequencing technologies available now and in the future. VESPA is compatible with existing bacterial and fungal pipelines, with metabarcoding of all three taxa run on the same sequencing platform. Addition of VESPA to established protocols for characterizing bacterial microbiomes and mycobiomes could have far reaching benefits. For example, it has been suggested that studies of the human gut microbiome should routinely incorporate analyses of eukaryotic diversity in order to capture overall microbial community function⁵. VESPA can provide this missing eukaryotic component and thereby enable cross-kingdom characterization of microbial ecosystem structure and function, opening new avenues for basic and applied research.

### Materials and Methods

### Methods Review and New Method Design

Literature searches were performed in January 2021 and updated in January 2023. Search terms or combinations of search terms including “Metagenomics,” “Metagenomic barcoding,” “Metabarcoding,” “Targeted amplicon deep sequencing,” “Eukaryotic microbiome,” “Gastrointestinal,” “Gut,” “Parasite,” and “18S” were used to query PubMed, Web of Science, and Google Scholar. Results were manually evaluated for relevance and details were compiled in an excel spreadsheet. We identified 96 studies including reviews and methods papers, 54 of which were primary research on vertebrate-associated eukaryotes. We chose to focus on 18S because in previous metabarcoding studies, non-coding genes outperformed coding genes^{36,48}, 18S has islands of conserved sequence interspersed with areas of high entropy (hypervariable regions), allowing broad priming for coverage and diverse amplicons for resolution⁴⁹, and database coverage for 18S is higher than for other loci⁵⁰. Of the 9 hypervariable 18S regions, V4 has the highest taxonomic resolution³⁷, so we focused on this region and identified 22 sets of published V4 primers (Table 5).

TABLE 5
18S primers used in this study

| | | | | SEQ | |
|---|---|---|---|---|---|
| | | | | ID | |
| Primer Name | Reference | F/R/B | Sequence (5′-3′) | NO: | Region |
| E572F | Comeau 2011 | F | CYGCGGTAATTCCAGCTC | 15 | V4 |
| E1009R | Comeau 2011 | R | AYGGTATCTRATCRTCTTYG | 16 | V4 |
| PNA mammal block | Mann 2020 | B | TCTTAATCATGGCCTCAGTT | 17 | V4 |
| 515f | Bates 2012 | F | GTGCCAGCMGCCGCGGTAA | 18 | V4 |
| 1119r | Bates 2012 | R | GGTGCCCTTCCGTCA | 19 | V4 |
| 18S-EUK581-F | Bower 2004 | F | GTGCCAGCAGCCGCG | 20 | V4 |
| 18S-EUK1134-R | Bower 2004 | R | TTTAAGTTTCAGCCTTGCG | 21 | V4 |
| TAReuk454FWD1 | Stoeck 2010 | F | CCAGCASCYGCGGTAATTCC | 22 | V4 |
| TAReukREV3 | Stoeck 2010 | R | ACTTTCGTTCTTGATYRA | 23 | V4 |
| V4r | Bradley 2016 | R | ACTTTCGTTCTTGAT | 24 | V4 |
| 3NDf | Cavalier-Smith 2009 | F | GGCAAGTCTGGTGCCAG | 25 | V4 |
| V4_euk_R1 | Brate 2010 | R | GACTACGACGGTATCTRATCRTCTTCG | 26 | V4 |
| V4_euk_R2 | Brate 2010 | R | ACGGTATCTRATCRTCTTCG | 27 | V4 |
| 18SV4_F | DeMone 2020 | F | GCCGCGGTAATTCCAGCTC | 28 | V4 |
| 18SV4_R | DeMone 2020 | R | ATYYTTGGCAAATGCTTTCGC | 29 | V4 |
| Giardia 18SV4_R | DeMone 2020 | R | ATACGGTGGTGTCTGATCGC | 30 | V4 |
| F-566 | Hadziavdic 2014 | F | CAGCAGCCGCGGTAATTCC | 31 | V4 |
| R-1200 | Hadziavdic 2014 | R | CCCGTGTTGAGTCAAATTAAGC | 32 | V4 |
| F-574 | Hadziavdic 2014 | F | GCGGTAATTCCAGCTCCAA | 33 | V4 |
| R-952 | Hadziavdic 2014 | R | TTGGCAAATGCTTTCGC | 34 | V4 |
| 616 | Hugerth 2014 | F | TTAAARVGYTCGTAGTYG | 35 | V4 |
| 574 | Hugerth 2014 | F | CGGTAAYTCCAGCTCYV | 36 | V4 |
| 563 | Hugerth 2014 | F | GCCAGCAVCYGCGGTAAY | 37 | V4 |
| 1132 | Hugerth 2014 | R | CCGTCAATTHCTTYAART | 38 | V4 |
| G3F1 | Krogsgaard 2018 | F | GCCAGCAGCCGCGGTAATTC | 39 | V4 |
| G3R1 | Krogsgaard 2018 | R | ACATTCTTGGCAAATGCTTTCGCAG | 40 | V4 |
| G4F3 | Krogsgaard 2018 | F | AGCCGCGGTAATTCCAGCTC | 41 | V4 |
| G4R3 | Krogsgaard 2018 | R | GGTGGTGCCCTTCCGTCAAT | 42 | V4 |
| G6F1 | Krogsgaard 2018 | F | TGGAGGGCAAGTCTGGTGCC | 43 | V4 |
| G6R1 | Krogsgaard 2018 | R | TACGGTATCTGATCGTCTTCGATCCC | 44 | V4 |
| 18S#1 | Machida 2012 | F | CTGGTGCCAGCAGCCGCGGYAA | 45 | V4 |
| Machida 2012 | Machida 2012 | R | TCCGTCAATTYCTTTAAGTT | 46 | V4 |
| MMSF | Sikder 2020 | F | GGTGCCAGCAGCCGCGGTA | 47 | V4 |
| MMSR | Sikder 2020 | R | CTTTAAGTTTCAGCTTTGC | 48 | V4 |
| Nem18SlongF | Wood 2013 | F | CAGGGCAAGTCTGGTGCCAGCAGC | 49 | V4 |
| Nem 18SlongR | Wood 2013 | R | GACTTTCGTTCTTGATTAATGAA | 50 | V4 |
| 9F | This study | F | CTGGTGCCAGCAGCCGCGG | 1 | V4 |
| 13F | This study | F | TGGTGCCAGCAGCCGCGG | 2 | V4 |
| 29F | This study | F | AGCAGCCGCGGTAATTCC | 3 | V4 |
| 2-2bF | This study | F | TGGTGCCAGCASCCGCG | 4 | V4 |
| 21b8R + 4 | This study | R | TCCGTCAATTYCTTNAASTTTC* | 6 | V4 |
| EukA_F | Medlin 1988 | F | AACCTGGTTGATCCTGCCAGT | 51 | 5′ terminus |
| EukB_R | Medlin 1988 | R | TGATCCTTCTGCAGGTTCACCTAC | 52 | 3′ terminus |
| 1520_R | Lopez-Garcia 2003 | R | CYGCAGGTTCACCTAC | 53 | 3′ terminus |
| V3Mod_F | This study (modified | F | CCGGAGAGRGAGCMTKAG | 54 | 5′ terminus |
| | from Flaherty 2018) | | | | |
| EukBshort_R | This study (modified | R | CCTTCCGCAGGTTCACCTAC | 55 | 3′ terminus |
| | from Medlin 1988) | | | | |
| LAOEukF | This study | F | CTGGTTGATCCTGCCAGTAKT | 56 | 5′ terminus |
| LAOEuk2F | This study | F | CTGGTTGATCCTGCCAGT | 57 | 5′ terminus |
| LAO18SF | This study | F | CGCGAANGGCTCATTANAWCAGC | 58 | 5′ terminus |
| LAOGiarF | This study | F | ACGGCTCAGGACAACGGTT | 59 | 5′ terminus |
| LAO1498R | This study | R | GGTTCACCTACGGANACCTTGTTA | 60 | 3′ terminus |
| LAOECR | This study | R | TCGTCTTCTCAGCGCCGGT | 61 | 3′ terminus |
| LAOEntCrypF | This study | F | GATTAAGCCATGCATGTSTAAG | 62 | 5′ terminus |
| LAO380F | This study | F | GGTTCGACTCCGGAGAG | 63 | 5′ terminus |
| LAOTW2F | This study | F | TGGATAACTGTAATRACTCT | 64 | 5′ terminus |
| LAOTW3R | This study | R | GACCTYACTAAACCATTCAATC | 65 | 3′ terminus |

F, Forward primer; R, Reverse primer; B, Blocking primer
*The N shown in bold in SEQ ID NO: 5 may be I, A, T, C, or G. While the 21b8R primer used in this example contained an I (5-deoxyinosine) at this position, we achieved similar results with a 1:1:1:1 mix of 21b8R variants comprising an A, T, C, or G at this position. Note: 5-deoxyinosine is a “universal” base that can base pair with A, T, C, or G.

We also designed new 18S V4 primers with the goal of amplifying all eukaryotic endosymbiont groups with little to no prokaryotic complementarity. We began by creating a database of parasite/commensal 18S rRNA sequences containing representatives from all phylogenetic lineages containing at least one vertebrate-associated eukaryotic endosymbiont. We downloaded sequences from all known groups of endoparasites/endosymbionts from NCBI Genbank⁵¹ or the SILVA 138.1 Small Subunit rRNA Non-Redundant Reference Database (n=510,508 total accessions^{50,52}. SILVA Ref NR hereafter) at a depth of one species per genus, beginning with the Centers for Disease Control's “Alphabetical Index of Parasitic Diseases”⁵³. To ensure broad coverage of commensals, zoonoses, and novel organisms we added non-pathogenic protozoans of humans⁵⁴, parasites/commensals of great apes⁵⁵, and parasites of veterinary importance⁵⁶. We then used MUSCLE⁵¹ implemented in MEGA 11⁵⁸ to align the resulting 658 full-length 18S sequences, which covered a broad range of pathogenicity, vertebrate hosts, and tissue tropisms. To identify candidate conserved regions, we utilized the Arb software suite⁵⁹, and the ecoPrimers function in OBItools⁶⁰, with manual inspection and adjustment as needed. We then extracted every 16-20-mer candidate sequence within those regions and tested them for taxonomic coverage against SILVA Ref NR using the SILVA TestProbe and TestPrime tools⁶¹. Candidate primers with high overall complementarity were manually adjusted for maximum coverage.

We aimed to avoid degeneracy as it has been shown to create bias in 18S V4 amplification³⁷ and succeeded in the forward primer. Degeneracy was required in the reverse primer, although not in the four terminal 3′ nucleotides. Furthermore, of the three degenerate positions in the reverse primer, no targeted groups required all three degeneracies, and most required just one. To increase homogeneity and avoid potential biases against rare sequences, we used 5-deoxyinosine in the four-fold degenerate position instead of N, thereby limiting our reverse primer mixture to four distinct oligonucleotides⁶². (Note: Inosine is considered a ‘universal base’ or a ‘degenerate base’ because it has the ability to pair indiscriminately with each of the four standard nucleotide bases, adenine (A), cytosine (C), guanine (G), and thymine (T), via two hydrogen bonds.)

The forward region identified for priming had higher GC content than the reverse region, so we forewent the standard guidelines for GC content and melting temperature differences in order to prioritize coverage, with the knowledge that we could later add locked nucleic acids (LNAs) to modify the melting temperature if needed⁶³. In the end, this modification was not necessary because the DNA polymerase for PCR (described below) tolerates a wide melting temperature range and has a universal annealing temperature regardless of primer sequence. In total we designed four forward primers and one reverse primer (Table 5) for further testing.

### Testing Metabarcoding Methods for Taxonomic Coverage Using in Silico PCR

For the initial analysis of published protocols for taxonomic coverage, we used locus-specific sequences (i.e., not including linkers, adapters, or barcode elements) from all 22 18S V4 primer sets identified in our literature search (Table 5). In silico PCR of SILVA Ref NR was performed using the TestPrime tool allowing for a single mismatch and a mismatch-free two base pair 3′ window. For this analysis, “helminth” accessions included Acanthocephala (n=66), Nematoda (n=2,170), and Platyhelminthes (n=1,993) and “protozoa” accessions included Amoebozoa (n=1,148), Discoba (n=1,032), Excavata (n=389), Alveolata (n=9,140), and Stramenopiles (n=3,556). In two cases where multiple primer sets were used in combination (Krosgaard—three sets and DeMone—two sets), we tested each set individually and conservatively estimated coverage by reporting only the highest percentage for each taxon. Primer sets with >5% coverage of off-target prokaryote groups (archaea and bacteria) were not analyzed further (n=4 sets).

In silico PCR was then used to evaluate the published primer sets remaining (n=18) alongside our new candidate primers (n=4; Table 5). At this stage, we filtered target sequences to contain only parasites of vertebrates because the inclusion of environmental/free-living organisms can distort parasite coverage metrics. Specifically, we split clades that contained both free-living organisms and parasites of invertebrate hosts (e.g., Rhabditida and Entamoeba) into higher-resolution, curated groups. We included free-living, opportunistic parasites of clinical importance, including Balamuthia mandrillaris and Naegleria fowleri, and we excluded sequences whose label in the SILVA database was incorrect (i.e., the taxonomy string associated with the record did not match the phylogenetic placement in the guide tree; n=14). Coverage metrics were normalized to eligible accession numbers, which were similar across primer sets because of similar priming locations in the V4 region. We compared taxonomic coverage for primer sets using the TestPrime tool⁶¹ and SILVA Ref NR^{50,52} allowing for a single mismatch with a mismatch-free two base pair 3′ window. Primers with ≤50% overall mean coverage of target groups and methods that required more than a single primer set were not considered further.

### Testing Metabarcoding Methods for On-Target Amplification Using Purified DNA

We assessed amplification success of the remaining 4 newly designed and 8 published primer sets across parasite groups using 22 genomic DNA (gDNA) isolates from single vertebrate endoparasites as template for PCR. Samples were obtained from reputable reagent repositories and expert parasitologists (for sample details, including sources, see Table 6) either as purified DNA or whole organisms. gDNA from whole worms and pelleted protozoal cultures were extracted using the DNeasy Blood and Tissue Kit (Qiagen, Hilden, Germany) using 0.2 g of starting material, eluted in Qiagen buffer AE, and stored at −20° C. PCR conditions were as follows: 1×Platinum II Hot Start PCR MasterMix (ThermoFisher, Waltham, Massachusetts, USA), 0.2 μM forward primer with Nextera adapter, 0.2 μM reverse primer with Nextera adapter, ThermoFisher 0.2×Platinum II GC Enhancer, 0.8 ng/μl gDNA in a total 12.5 μl reaction; 94° C. for 2 minutes, 30 cycles of [94° C. for 15 seconds, 60° C. for 15 seconds, 68° C. for 15 seconds], and hold at 4° C. Products were electrophoresed on a 1.5% agarose gel with SYBR gold DNA dye (ThermoFisher) and a 1 kb DNA size standard. Amplification was scored by band presence on an agarose gel upon visualization under UV illumination with a GelDoc XR imager (BioRad, Hercules, California, USA).

TABLE 6
Parasite specimens and sources

| Organism | Sample type | Source | Catalog # |
|---|---|---|---|
| Echinorhynchus | Whole adult | UW Madison School of | NA |
| salmonis | worms | Veterinary Medicine, Dr. Tony | |
| Hymenolepis diminuta | Whole adult | UW Madison School of | NA |
| | worms | Veterinary Medicine, Dr. Timothy | |
| Taenia hydatigena | Cysts | Wisconsin Veterinary Diagnostic | NA |
| | | Lab | |
| Bertiella studeri | Proglottids | UW Madison School of | NA |
| | | Veterinary Medicine, Dr. Tony | |
| Schistosoma mansoni | DNA | BEI Resources | NR-28911 |
| Strain NMRI | | | |
| Ascaris suum | Whole adult | Wisconsin Veterinary Diagnostic | NA |
| | worms | Lab | |
| Dictyocaulus viviparous | Whole adult | Wisconsin Veterinary Diagnostic | NA |
| | worms | Lab | |
| Dirofilaria immitis | DNA | BEI Resources | NR-44348 |
| Strain Missouri 2005 | | | |
| Trichinella spiralis | DNA | USDA Animal Parasitic Diseases | NA |
| | | Laboratory | |
| Encephalitozoon | DNA | BEI Resources | NR-13510 |
| cuniculi | | | |
| Entamoeba histolytica | DNA | BEI Resources | NR-175 |
| Strain HK-9 | | | |
| Balamuthia mandrillaris | Axenic culture | BEI Resources | NR-46452 |
| CDC: V188 | | | |
| Acanthamoeba sp. | DNA | BEI Resources | NR-45611 |
| Strain CDC: 12741:1 | | | |
| Naegleria fowleri | Axenic culture | BEI Resources | NR-46494 |
| Strain CDC: V414 | | | |
| Leishmania major | DNA | BEI Resources | NR-48764 |
| Strain NIH SD | | | |
| Trypanosoma cruzi | DNA | BEI Resources | NR-50238 |
| Strain G | | | |
| Giardia lamblia | DNA | BEI Resources | NR-15894 |
| Strain WB clone C6 | | | |
| Plasmodium falciparum | DNA | BEI Resources | MRA-398 |
| Strain D6 | | | |
| Babesia sp. | DNA | BEI Resources | NR-50663 |
| Strain MO1 | | | |
| Toxoplasma gondii | DNA | UW Madison Department of | NR-33509 |
| | | Medical Microbiology and | |
| | | Immunology, Dr. Laura Knoll | |
| Cryptosporidium | DNA | BEI Resources | NR-2520 |
| hominis | | | |
| Blastocystis hominis | DNA | ATCC (American Type Culture | 50608 |
| Strain BT1 | | Collection) | |

NA, not applicable.

### Testing Metabarcoding Methods for Amplification Bias Using a Community Standard

Preliminary metabarcoding experiments using mixes of gDNA from single parasites demonstrated a non-linear relationship between DNA input and sequence read abundance, likely due to rRNA copy number variation⁶⁴. We addressed this issue by extracting, amplifying, and cloning parasite DNA from 16 vouchered parasite specimens from verified sources or identified by experts (Table 6). 18S rDNA sequences were amplified with full-length universal or group-specific primers (see Table 5 and Table 7) using Qiagen HotStar Plus Taq DNA polymerase according to manufacturer's instructions. Products were verified for size on an agarose gel and Sanger sequenced. Correct 18S sequences were cloned into a pCR4-TOPO vector using a TOPO TA Cloning Kit for Sequencing (Invitrogen, Waltham, Massachusetts, USA) and Invitrogen One Shot competent cells according to manufacturer's instructions. Colonies were screened by PCR and Sanger sequencing. Plasmid DNA (plDNA) extracted from verified transformants was mixed at equimolar ratios to create the equimolar EukMix community standard reagent. This strategy assures equal 18S copy number input among organisms, which, in the case of amplicon sequencing, enables assessment of primer bias and potential of the assays to yield quantitative data⁶⁵.

TABLE 7
Equimolar EukMix components and
full-length 18S cloning primers

| | Organism | FWD primer* | REV primer* |
|---|---|---|---|
| 1 | Echinorhynchus salmonis | EukA_F | EukB_R |
| 2 | Hymenolepis diminuta | LAOTW2F | LAOTW3R |
| 3 | Ascaris suum | LAO18SF | LAO1498R |
| 4 | Dirofilaria immitis | LAO18SF | LAO1498R |
| 5 | Trichinella spiralis | V3mod_F | EukBshort_R |
| 6 | Encephalitozoon cuniculi | V3mod_F | LAOECR |
| 7 | Entamoeba histolytica | LAOEuk2F | EukB_R |
| 8 | Balamuthia mandrillaris | EukA_F | EukB_R |
| 9 | Naegleria fowleri | LAO380F | LAO1498R |
| 10 | Giardia intestinalis | LAO380F | EukB_R |
| 11 | Leishmania major | LAOEukF | EukB_R |
| 12 | Plasmodium falciparum | EukA_F | EukB_R |
| 13 | Babesia sp. strain MO1 | EukA_F | EukB_R |
| 14 | Toxoplasma gondii | EukA_F | EukB_R |
| 15 | Cryptosporidium hominis | EukA_F | LAO1498R |
| 16 | Blastocystis hominis | LAOEukF | LAO1498R |

*See Table 5 for primer sequences and references

Metabarcoding using new and published primer sets was performed in triplicate with community standard as starting material using the procedure described below. Resulting sequencing reads were filtered for quality using a cutoff of Q=30 and mapped to a database containing full-length 18S sequences of clones comprising the EukMix mock community using a mapping stringency of 99% similarity and 99% length fraction in CLC genomics workbench v. 10.2 (Qiagen). The resulting abundances for each community standard component were used to calculate evenness metrics in R v. 3.6.3, and GraphPad Prism v. 8.4.3 was used for graphing data and for statistical analyses.

### VESPA Compared to Microscopy

Sample collection: Clinical samples used in this work were excess material from concluded studies that had been previously evaluated for eukaryotic endosymbionts using microscopy. Human fecal samples had been collected from communities on the southern Venezuelan border with Brazil⁶⁶. Non-human primate fecal samples were collected from semi-free ranging Nigerian red capped mangabeys (Cercocebus torquatus) in a sanctuary⁶⁷. Appropriate IRB approvals (IVIC IRB #DIR-0609/1542/2015) and IACUC protocols were obtained by each collaborator and all samples were completely de-identified prior to use.

Microscopy: Microscopic analyses of non-human primate and human feces were performed as previously described⁶⁸. Briefly, one gram of formalin preserved feces was concentrated via formalin-ethyl acetate sedimentation⁶⁷ and the sediment was examined in its entirety at ×10 objective light magnification for gastrointestinal parasites by an expert parasitologist. Additionally, one drop of sediment from each sample was examined at ×40 objective light magnification for identification of protozoa.

Genomic DNA isolation: Human fecal samples were processed to remove bacteria and debris as previously described⁶⁹. Briefly, feces were diluted in PBS (0.2 M phosphate-buffered saline, pH 7.2), homogenized, filtered through sterile four-ply cotton gauze, pelleted for 5 min at 300×g, resuspended in molecular grade water and layered on top of a 1.5 M sucrose solution. After centrifugation for 10 min at 1,700×g the interphase was collected and the process was repeated with a 0.75 M sucrose gradient. The resulting pellet was collected, washed in PBS, and resuspended in 2 ml of molecular-grade water. 0.2 ml of the resulting sample was used as starting material for phenol: chloroform: isoamyl alcohol (25:24:1) DNA extraction, eluted in IDTE buffer and stored at −20° C.

Non-human primate fecal samples in 1:1 RNAlater nucleic acid preservation solution (ThermoFisher) were thawed on ice and homogenized by vortexing prior to transferring 0.2 g of homogenate to bead beating tubes (for a total of 0.1 g fecal material) for extraction using the Qiagen DNeasy PowerLyzer PowerSoil kit. gDNA was eluted in Qiagen C6 buffer and stored at −20° C.

Metabarcoding: See VESPA Protocol for step-by-step instructions. For compatibility of sequencing libraries across primer sets and amplicon library types, we created a 2-step Illumina Nextera-based protocol that does not require custom sequencing primers to be added to the sequencing cartridge. Primers for the first (amplicon) PCR consist of a locus-specific sequence (see VESPA Protocol and Table 5 for locus-specific primer sequences) with Nextera adapter sequences attached at the 5′ end: F-TCGTCGGCAGCGTCAGATGTGTATAAGAGACAG (SEQ ID NO: 66) and R-GTCTCGTGGGCTCGGAGATGTGTATAAGAGACAG (SEQ ID NO: 67). A second, limited cycle (indexing) PCR was then used to add Nextera indexing primers to both ends. Note that Platinum II MasterMix (ThermoFisher) has a universal annealing temperature of 60° C. regardless of primer melting temperature. PCRs were run in triplicate with the following conditions: ThermoFisher 1×Platinum II Hot Start PCR MasterMix, 0.2 μM forward primer with Nextera adapter, 0.2 μM reverse primer with Nextera adapter, 0.2×ThermoFisher Platinum II GC Enhancer, 0.8 ng/μl gDNA in a total 12.5 μl reaction; 94° C. for 2 minutes, 30 cycles of [94° C. for 15 seconds, 60° C. for 15 seconds, 68° C. for 15 seconds], and hold at 4° C. Triplicate reactions were then pooled and amplicons were cleaned using Ampure XP beads (Beckman Coulter, Brea, California, USA) then used as template for indexing PCR as follows: 1×KAPA HiFi HotStart ReadyMix (Roche, Basel, Switzerland), 1×Nextera Unique Dual Index primers (Illumina, San Diego, California, USA), 1 μl of clean amplicons in a total 12.5 μl reaction; 95° C. for 3 minutes, 10 cycles of [95° C. for 30 seconds, 55° C. for 30 seconds, 72° C. for 30 seconds], 72° C. for 5 minutes, and hold at 4° C. Indexed libraries were cleaned using Ampure XP beads (Beckman Coulter) assessed for concentration on a Qubit fluorometer (ThermoFisher), and pooled for sequencing on an Illumina MiSeq with 300×300 cycle chemistry using default index and sequencing read primers and 10-20% PhiX.

Data processing and Bioinformatics: We processed reads from our final two VESPA data sets with both QIIME 2⁷⁰ and DADA2 v. 1.16.0⁷¹ in the R environment v. 3.6.3 and found that, while results were similar, DADA2 was more user-friendly (i.e., did not require installation of new software, required less steps, and was implementable within a familiar computing environment). Read files were converted to vectors and filtered for quality using the filterAndTrim command with default settings plus modifiers to remove primers (trimLeft=c(18,20)), residual PhiX reads (rm.phix=TRUE), and short sequences (minLen=100). Error rate for forward and reverse reads were calculated using the learnErrors command, data were dereplicated using the derepFastq command, and sequence variants were inferred using the dada command. Read pairs were merged using the mergePairs command with justConcatenate=TRUE and chimeras were removed using the removeBimeraDenovo command with default parameters. Taxonomy assignments were made using the assignTaxonomy command and the PR2 version 4.14.0 database, which contains 18S and 16S sequences at species-level resolution. For comparison we also tested 2 other taxonomy databases: v132 which includes all eukaryotic organisms from the SILVA v132 database and v128 which includes all eukaryotic organisms from the SILVA v128 database plus corrected species labels for Blastocystis and additional Entamoeba sequences. However, we found that the PR2 database returned higher numbers of fully assigned ASVs. Any ASVs not assigned taxonomy using the PR2 database were queried against the full NCBI nucleotide database on Sep. 3, 2022 using MegaBLAST⁷² with default parameters.

### Data Availability

Sequence data that support the findings of this study have been deposited in the National Center for Biotechnology Information (NCBI) Sequence Read Archive with BioProject ID PRJNA944233 and BioSample accessions SAMN33744948 to SAMN33744999.

### VESPA Protocol:

### Contents

1—Starting material
2—gDNA extraction
3—18S V4 Amplicon PCR
4—Amplicon cleanup
5—Indexing PCR
6—Library cleanup
7—Quantification and size determination
8—Pooling and sequencing

### I—Starting Material

Starting material can be fresh, freshly frozen (no buffer), or stored ˜1:1 in RNA later.
Sample types tested: feces, vomit, stomach contents, intestine tissue, intestine contents, environmental, entamoeba cysts, whole helminths, and tapeworm proglottids/cysts.
   2—gDNA Extraction
Use Qiagen DNeasy PowerLyzer PowerSoil Kit (catalog #12855-5) according to manufacturer's instructions.
Weigh out up to 0.20 g of input feces or 0.25 g of input for all other sample types.
Elute in 100 μl C6 buffer (included in kit) and store at −20° C.

### 3—18S V4 Amplicon PCR

Set up amplicon PCR reactions in triplicate.
Use Invitrogen Platinum II Hot Start 2×PCR Master Mix (Catalog #14000012) with the reaction conditions (Table 8) and cycling conditions (Table 9) shown below.
Use a forward primer selected from SEQ ID NOs: 1-4 and a reverse primer selected from SEQ ID NOs: 5-8. Include Nextera adapter sequences, as demonstrated below.

TABLE 8
Reaction conditions for 18S V4 amplicon PCR

| | | |
|---|---|---|
| Reaction component | Final Conc. | 1 × 12.5 μl rxn. (μl) |

| | | |
|---|---|---|
| 2X Platinum II HotStart PCR Master Mix* | 1X | 6.0 |

| | | | |
|---|---|---|---|
| 10 μM Forward primer | 0.2 | μM | 0.25 |
| 10 μM Reverse primer | 0.2 | μM | 0.25 |

| | | |
|---|---|---|
| Platinum II GC Enhancer* | NA | 2.5 |
| Nuclease-free water* | NA | 2.5 |

| | | | |
|---|---|---|---|
| ~10 ng/μl gDNA | 0.8 | ng/μl | 1.0 |

12.5 μl

*Included in Master Mix Kit

TABLE 9
Cycling conditions for 18S V4 amplicon PCR

| Step | Temp ° C. | Time | | Cycles |
|---|---|---|---|---|
| Activation | 94 | 2 | min | 1 |
| Denaturation | 94 | 15 | sec | 30 |
| Annealing | 60 | 15 | sec | |
| Extension | 68 | 15 | sec | |

| | | |
|---|---|---|
| Final hold | 4 | hold |

Primers (Nextera adapter sequence-locus-specific primer sequence)
Forward: 29F
(SEQ ID NO: 66-SEQ ID NO: 3)
TCGTCGGCAGCGTCAGATGTGTATAAGAGACAGAGCAGCCGCGGTAATTCC
Reverse: 21b8R + 4
(SEQ ID NO: 67-SEQ ID NO: 6)
GTCTCGTGGGCTCGGAGATGTGTATAAGAGACAGTCCGTCAATTYCTTNAASTTTC

### 4—Amplicon Cleanup

Use Beckman Coulter Ampure XP beads (catalog #A63880) and magnetic particle separator (MPC).
Always make 75% ethanol immediately prior to use.
1—Shake Ampure XP beads at room temperature for >30 minutes prior to use.
2—Pool all 3 PCR reactions into a single plate or tube and mix by pipetting (˜37.5 μl).
3—Remove 7.5 μl and store at −20° C. if you would like to visualize bands on a gel (˜30 μl).
4—Add AMPure XP beads for 0.8× RATIO (e.g., 24 μl beads per 30 μl product).
5—Gently pipette up and down 15 times.
6—Incubate at room temperature for 5 minutes.
7—Put tubes on MPC and incubate at room temperature for 2 minutes.
8—Remove and discard supernatant.
9—With tubes on MPC, add 175 μl of 75% ethanol.
10—Wait >1 minute.
11—Remove and discard supernatant.
12—Add 175 μl of 75% ethanol.
13—Wait >1 minute.
14—Remove and discard supernatant.
15—Remove all ethanol with P20 tips.
16—With tubes on MPC, let the pellet air-dry for 5 minutes.
17—Add 47 μl of Tris pH 8.5.
18—Remove tubes from MPC and gently pipette up and down to resuspend beads.
19—Incubate at room temperature for 2 minutes.
20—Put tubes on MPV and incubate at room temperature for 2 minutes.
21—Carefully transfer 45 μl of supernatant to a new PCR tubes or plate.

### 5—Indexing PCR

Set up indexing PCR reactions on ice.
Use Roche KAPA HiFi HotStart ReadyMix (catalog #KK2601) and IDT for Illumina Nextera DNA Unique Dual Indexes (catalog #20027215) with the reaction conditions (Table 10) and cycling conditions (Table 11) shown below.

TABLE 10
Reaction conditions for indexing PCR

| Reaction component | 1 × 12.5 μl rxn. (μl) |
|---|---|
| 2X KAPA HiFi HotStart ReadyMix | 6.0 |
| Nextera Unique Dual Index | 2.5 |
| Nuclease-free water | 3.0 |
| Clean amplicons in Tris pH 8.5 | 1.0 |
| | 12.5 μl |

TABLE 11
Cycling conditions for indexing PCR

| Step | Temp ° C. | Time | | Cycles |
|---|---|---|---|---|
| Activation | 95° C. | 3 | min | 1 |
| Denaturation | 95° C. | 30 | sec | 10 |
| Annealing | 55° C. | 30 | sec | |
| Extension | 72° C. | 30 | sec | |
| Final extension | 72° C. | 5 | min | 1 |

| | | |
|---|---|---|
| Final hold | 4° C. | hold |

### 6—Library Cleanup

Use Beckman Coulter Ampure XP beads (catalog #A63880) and magnetic particle separator (MPC).
Always make 75% Ethanol immediately prior to use.
1—Shake Ampure XP beads at room temperature for >30 minutes prior to use.
2—Add AMPure XP beads for 0.8× RATIO (e.g., 9.6 μl beads per 12.5 μl PCR product).
3—Gently pipette up and down 15 times.
4—Incubate at room temperature for 5 minutes.
5—Put tubes on MPC and incubate at room temperature for 2 minutes.
6—Remove and discard supernatant.
7—With tubes on MPC, add 175 μl of 75% ethanol.
8—Wait >1 minute.
9—Remove and discard supernatant.
10—Add 175 μl of 75% ethanol.
11—Wait >1 minute.
12—Remove and discard supernatant.
13—Remove all ethanol with P20 tips.
14—With tubes on MPC, let the pellet air-dry for 5 minutes.
15—Add 22 μl of Tris pH 8.5.
16—Remove tubes from MPC and gently pipette up and down to resuspend beads.
17—Incubate at room temperature for 2 minutes.
18—Put tubes on MPV and incubate at room temperature for 2 minutes.
19—Carefully transfer 20 μl of supernatant to a new PCR tubes or plate.

### 7—Quantification and Size Determination

Use Invitrogen Qubit Fluorimeter and dsDNA High-Sensitivity Assay Kit (catalog #Q33230) and Agilent Bioanalyzer and Agilent High Sensitivity DNA Kit (catalog #5067-4626) according to manufacturer's instructions.
Measure the concentration of each library using a Qubit fluorometer and 3 μl of each library.
Measure the size of each library or a representative subset of libraries using an Agilent Bioanalyzer and 1 μl of a 1 ng/μl dilution (in Tris pH 8.5) of the library for a total of 1 ng.

### 8—Pooling and Sequencing

Requirements for core facility submission/in-house sequencing will determine pooling specifics.
Run on an Illumina MiSeq instrument, 300×300 cycle chemistry, and add 10-20% PhiX.

### REFERENCES

1. Whipps J M L, K.; Cooke, R. C. Mycoparasitism and plant disease control. In: Burge M N, ed. Fungi in biological control systems. Manchester University Press; 1988:161-187:chap 9.
2. Konopka A. What is microbial community ecology?Isme J. November 2009; 3(11):1223-30. doi:10.1038/ismej.2009.88
3. Gilbert J A, Blaser M J, Caporaso J G, Jansson J K, Lynch S V, Knight R. Current understanding of the human microbiome. Nat Med. Apr. 10 2018; 24(4):392-400. doi:10.1038/nm.4517
4. Pepper J W, Rosenfeld S. The emerging medical ecology of the human gut microbiome. Trends Ecol Evol. July 2012; 27(7):381-4. doi:10.1016/j.tree.2012.03.002
5. Clemente J C, Ursell L K, Parfrey L W, Knight R. The impact of the gut microbiota on human health: an integrative view. Cell. Mar. 16 2012; 148(6):1258-70. doi:10.1016/j.cell.2012.01.035
6. Laforest-Lapointe I, Arrieta M C. Microbial eukaryotes: a missing link in gut microbiome studies. mSystems. March-April 2018; 3(2)doi:10.1 128/mSystems.00201-17
7. Kodio A, Menu E, Ranque S. Eukaryotic and prokaryotic microbiota interactions. Microorganisms. Dec. 17 2020; 8(12)doi:10.3390/microorganisms8122018
8. Avramenko R W, Redman E M, Lewis R, Yazwinski T A, Wasmuth J D, Gilleard J S. Exploring the gastrointestinal “Nemabiome”: deep amplicon sequencing to quantify the species composition of parasitic nematode communities. Plos One. Dec. 2 2015; 10(12)
9. Matijasic M, Mestrovic T, Paljetak H C, Peric M, Baresic A, Verbanac D. Gut microbiota beyond bacteria-mycobiome, virome, archaeome, and eukaryotic parasites in IBD. Int J Mol Sci. Apr. 11 2020; 21(8)doi:10.3390/ijms21082668
10. Kohler J R, Hube B, Puccia R, Casadevall A, Perfect J R. Fungi that infect humans. Microbiol Spectr. June 2017; 5(3)doi:10.1128/microbiolspec.FUNK-0014-2016
11. Tedersoo L, Bahram M, Zinger L, et al. Best practices in metabarcoding of fungi: from experimental design to results. Mol Ecol. May 2022; 31(10):2769-2795. doi:10.1111/mec.16460
12. Vossbrinck C R, Debrunner-Vossbrinck B A. Molecular phylogeny of the Microsporidia: ecological, ultrastructural and taxonomic considerations. Folia Parasitol (Praha). May 2005; 52(1-2):131-42; discussion 130. doi:10.14411/fp.2005.017
13. Dobell C. The discovery of the intestinal protozoa of man. Proc R Soc Med. 1920; 13 (Sect Hist Med):1-15.
14. Momcilovic S, Cantacessi C, Arsic-Arsenijevic V, Otranto D, Tasic-Otasevic S. Rapid diagnosis of parasitic diseases: current scenario and future needs. Clin Microbiol Infect. March 2019; 25(3):290-309. doi:10.1016/j.cmi.2018.04.028
15. Ricciardi A, Ndao M. Diagnosis of parasitic infections: what's going on?J Biomol Screen. January 2015; 20(1):6-21. doi:10.1177/1087057114548065
16. Nadler S A, G P DEL. Integrating molecular and morphological approaches for characterizing parasite cryptic species: implications for parasitology. Parasitology. November 2011; 138(13):1688-709. doi:10.1017/S003118201000168X
17. Jackson T F. Entamoeba histolytica and Entamoeba dispar are distinct species; clinical, epidemiological and serological evidence. Int J Parasitol. January 1998; 28(1):181-6. doi:10.1016/s0020-7519(97)00177-x
18. Fotedar R, Stark D, Beebe N, Marriott D, Ellis J, Harkness J. PCR detection of Entamoeba histolytica, Entamoeba dispar, and Entamoeba moshkovskii in stool samples from Sydney, Australia. J Clin Microbiol. March 2007; 45(3):1035-7. doi:10.1128/JCM.02144-06
19. Cristescu M E. From barcoding single individuals to metabarcoding biological communities: towards an integrative approach to the study of global biodiversity. Trends Ecol Evol. October 2014; 29(10):566-71. doi:10.1016/j.tree.2014.08.001
20. D'Amore R, Ijaz U Z, Schirmer M, et al. A comprehensive benchmarking study of protocols and sequencing platforms for 16S rRNA community profiling. BMC Genomics. Jan. 14 2016; 17:55. doi:10.1186/s12864-015-2194-9
21. Nilsson R H, Anslan S, Bahram M, Wurzbacher C, Baldrian P, Tedersoo L. Mycobiome diversity: high-throughput sequencing and identification of fungi. Nat Rev Microbiol. January 2019; 17(2):95-109. doi:10.1038/s41579-018-0116-y
22. Amaral-Zettler L A, McCliment E A, Ducklow H W, Huse S M. A method for studying protistan diversity using massively parallel sequencing of V9 hypervariable regions of small-subunit ribosomal RNA genes. PLoS One. Jul. 27 2009; 4(7):e6372. doi:10.1371/journal.pone.0006372
23. Parfrey L W, Walters W A, Lauber C L, et al. Communities of microbial eukaryotes in the mammalian gut within the context of environmental eukaryotic diversity. Frontiers in Microbiology. Jun. 19 2014; 5
24. Mann A E, Mazel F, Lemay M A, et al. Biodiversity of protists and nematodes in the wild nonhuman primate gut. Isme J. February 2020; 14(2):609-622. doi:10.1038/s41396-019-0551-4
25. Maritz J M, Rogers K H, Rock™, et al. An 18S rRNA workflow for characterizing protists in sewage, with a focus on zoonotic trichomonads. Microb Ecol. November 2017; 74(4):923-936. doi:10.1007/s00248-017-0996-9
26. Jarman S N, McInnes J C, Faux C, et al. Adelie penguin population diet monitoring by analysis of food DNA in scats. Plos One. Dec. 16 2013; 8(12)doi:ARTN e8222710.1371/journal.pone.0082227
27. Bhadury P, Austen M C. Barcoding marine nematodes: an improved set of nematode 18S rRNA primers to overcome eukaryotic co-interference. Hydrobiologia. March 2010; 641(1):245-251. doi:10.1007/s10750-009-0088-z
28. Avramenko R W, Bras A, Redman E M, et al. High species diversity of trichostrongyle parasite communities within and between Western Canadian commercial and conservation bison herds revealed by nemabiome metabarcoding. Parasites & Vectors. May 15 2018; 11doi:ARTN 29910.1186/s13071-018-2880-y
29. Avramenko R W, Redman E M, Lewis R, et al. The use of nemabiome metabarcoding to explore gastro-intestinal nematode species diversity and anthelmintic treatment effectiveness in beef calves. International Journal for Parasitology. November 2017; 47(13):893-902. doi:10.1016/j.ijpara.2017.06.006
30. Poissant J, Gavriliuc S, Bellaw J, et al. A repeatable and quantitative DNA metabarcoding assay to characterize mixed strongyle infections in horses. Int J Parasitol. February 2021; 51(2-3):183-192. doi:10.1016/j.ijpara.2020.09.003
31. Dollive S, Peterfreund G L, Sherrill-Mix S, et al. A tool kit for quantifying eukaryotic rRNA gene sequences from human microbiome samples. Genome Biol. Jul. 3 2012; 13(7):R60. doi:10.1186/gb-2012-13-7-r60
32. Krogsgaard L R, Andersen L O, Johannesen T B, et al. Characteristics of the bacterial microbiome in association with common intestinal parasites in irritable bowel syndrome. Clin Transl Gastroenterol. Jun. 19 2018; 9(6):161. doi:10.1038/s41424-018-0027-2
33. Gogarten J F, Calvignac-Spencer S, Nunn C L, et al. Metabarcoding of eukaryotic parasite communities describes diverse parasite assemblages spanning the primate phylogeny. Mol Ecol Resour. January 2020; 20(1):204-215. doi:10.1111/1755-0998.13101
34. Lamb P D, Hunter E, Pinnegar J K, Creer S, Davies R G, Taylor M I. How quantitative is metabarcoding: A meta-analytical approach. Molecular Ecology. 2019; 28(2):420-430.
35. Sergaki C, Anwar S, Fritzsche M, et al. Developing whole cell standards for the microbiome field. Microbiome. Aug. 9 2022; 10(1):123. doi:10.1186/s40168-022-01313-z
36. Marquina D, Andersson A F, Ronquist F. New mitochondrial primers for metabarcoding of insects, designed and evaluated using in silico methods. Mol Ecol Resour. January 2019; 19(1):90-104. doi:10.1111/1755-0998.12942
37. Bradley I M, Pinto A J, Guest J S. Design and evaluation of Illumina MiSeq-compatible, 18S rRNA gene-specific primers for improved characterization of mixed phototrophic communities. Applied and Environmental Microbiology. October 2016; 82(19):5878-5891. doi:10.1128/Aem.01630-16
38. Beermann A J, Werner M T, Elbrecht V, Zizka V M A, Leese F. DNA metabarcoding improves the detection of multiple stressor responses of stream invertebrates to increased salinity, fine sediment deposition and reduced flow velocity. Sci Total Environ. Jan. 1 2021; 750:141969. doi:10.1016/j.scitotenv.2020.141969
39. Bohmann K, Elbrecht V, Caroe C, et al. Strategies for sample labelling and library preparation in DNA metabarcoding studies. Mol Ecol Resour. May 2022; 22(4):1231-1246. doi:10.1111/1755-0998.13512
40. Song F, Kuehl J V, Chandran A, Arkin A P. A simple, cost-effective, and automation-friendly direct PCR approach for bacterial community analysis. mSystems. Oct. 26 2021; 6(5):e0022421. doi:10.1128/mSystems.00224-21
41. Albaina A, Aguirre M, Abad D, Santos M, Estonba A. 18S rRNA V9 metabarcoding for diet characterization: a critical evaluation with two sympatric zooplanktivorous fish species. Ecol Evol. March 2016; 6(6):1809-24. doi:10.1002/ece3.1986
42. Krehenwinkel H, Wolf M, Lim J Y, Rominger A J, Simison W B, Gillespie R G. Estimating and mitigating amplification bias in qualitative and quantitative arthropod metabarcoding. Sci Rep. Dec. 15 2017; 7(1):17668. doi:10.1038/s41598-017-17333-x
43. Deagle B E, Thomas A C, McInnes J C, et al. Counting with DNA in metabarcoding studies: how should we convert sequence reads to dietary data?Mol Ecol. January 2019; 28(2):391-406. doi:10.1111/mec.14734
44. Yu Z, Ito S I, Wong M K, et al. Comparison of species-specific qPCR and metabarcoding methods to detect small pelagic fish distribution from open ocean environmental DNA. PLoS One. 2022; 17(9):e0273670. doi:10.1371/journal.pone.0273670
45. Weerakoon K G, McManus D P. Cell-Free DNA as a diagnostic tool for human parasitic infections. Trends Parasitol. May 2016; 32(5):378-391. doi:10.1016/j.pt.2016.01.006
46. Maldonado A, Simoes R O, Luiz J S, Costa-Neto S F, Vilela R V. A new species of Physaloptera (Nematoda: Spirurida) from Proechimys gardneri (Rodentia: Echimyidae) from the Amazon rainforest and molecular phylogenetic analyses of the genus. J Helminthol. Jul. 24 2019; 94:e68. doi:10.1017/50022149X19000610
47. Abraham J S, Sripoorna S, Maurya S, Makhija S, Gupta R, Toteja R. Techniques and tools for species identification in ciliates: a review. Int J Syst Evol Microbiol. April 2019; 69(4):877-894. doi:10.1099/ijsem.0.003176
48. Macheriotou L, Guilini K, Bezerra T N, et al. Metabarcoding free-living marine nematodes using curated 18S and CO1 reference sequence databases for species-level taxonomic assignments. Ecol Evol. February 2019; 9(3):1211-1226. doi:10.1002/ece3.4814
49. Hadziavdic K, Lekang K, Lanzen A, Jonassen I, Thompson E M, Troedsson C. Characterization of the 18S rRNA gene for designing universal eukaryote specific primers. Plos One. Feb. 7 2014; 9(2)doi:ARTN e8762410.1371/journal.pone.0087624
50. Quast C, Pruesse E, Yilmaz P, et al. The SILVA ribosomal RNA gene database project: improved data processing and web-based tools. Nucleic Acids Res. January 2013; 41 (Database issue):D590-6. doi:10.1093/nar/gks1219
51. Benson D A, Cavanaugh M, Clark K, et al. GenBank. Nucleic Acids Res. Jan. 4 2018; 46(D1):D41-D47. doi:10.1093/nar/gkx1094
52. Yilmaz P, Parfrey L W, Yarza P, et al. The SILVA and “All-species Living Tree Project (LTP)” taxonomic frameworks. Nucleic Acids Res. January 2014; 42 (Database issue):D643-8. doi:10.1093/nar/gkt1209
53. Centers for Disease Control G H, Division of Parasitic Diseases and Malaria. Alphabetical Index of Parasitic Diseases. www.cdc.gov/parasites/az/index.html
54. Lukes J, Stensvold C R, Jirku-Pomajbikova K, Parfrey L W. Are human intestinal eukaryotes beneficial or commensals?Plos Pathogens. August 2015; 11(8)doi:ARTN e1005039 10.1371/journal.ppat.1005039
55. Modry D P, B. Petrielkova, K. Hasegawa, H. Parasites of apes an atlas of coproscopic diagnostics. vol 78. Frankfurt Contributions to Natural History/Frankfurter Beitrage zur Naturkunde. Edition Chimaira; 2018:198.
56. Taylor M A, Coop R L, Wall R. Veterinary parasitology. 4th edition. ed. John Wiley and Sons, Inc.; 2016:p.
57. Edgar R C. MUSCLE: multiple sequence alignment with high accuracy and high throughput. Nucleic Acids Res. 2004; 32(5):1792-7. doi:10.1093/nar/gkh340
58. Tamura K, Stecher G, Kumar S. MEGA11: Molecular Evolutionary Genetics Analysis version 11. Mol Biol Evol. Jun. 25 2021; 38(7):3022-3027. doi:10.1093/molbev/msab120
59. Ludwig W, Strunk O, Westram R, et al. ARB: a software environment for sequence data. Nucleic Acids Res. 2004; 32(4):1363-71. doi:10.1093/nar/gkh293
60. Riaz T, Shehzad W, Viari A, Pompanon F, Taberlet P, Coissac E. ecoPrimers: inference of new DNA barcode markers from whole genome sequence analysis. Nucleic Acids Res. November 2011; 39(21):e145. doi:10.1093/nar/gkr732
61. Klindworth A, Pruesse E, Schweer T, et al. Evaluation of general 16S ribosomal RNA gene PCR primers for classical and next-generation sequencing-based diversity studies. Nucleic Acids Res. Jan. 7 2013; 41(1):el. doi:10.1093/nar/gks808
62. Loakes D. Survey and summary: The applications of universal DNA base analogues. Nucleic Acids Res. Jun. 15 2001; 29(12):2437-47. doi:10.1093/nar/29.12.2437
63. Levin J D, Fiala D, Samala M F, Kahn J D, Peterson R J. Position-dependent effects of locked nucleic acid (LNA) on DNA sequencing and PCR primers. Nucleic Acids Res. 2006; 34(20):e142. doi:10.1093/nar/gkl756
64. Wang C, Zhang T, Wang Y, Katz L A, Gao F, Song W. Disentangling sources of variation in SSU rDNA sequences from single cell analyses of ciliates: impact of copy number variation and experimental error. Proc Biol Sci. Jul. 26 2017; 284(1859)doi:10.1098/rspb.2017.0425
65. Pinol J, Senar M A, Symondson W O. The choice of universal primers and the chatacteristics of the species mixtuer determine when DNA metabarcoding can be quantitative. Molecular Ecology Notes. 2018; 28:407-419.
66. Clemente J C, Pehrsson E C, Blaser M J, et al. The microbiome of uncontacted Amerindians. Sci Adv. Apr. 3 2015; 1(3)doi:10.1 126/sciadv.1500183
67. Friant S, Ziegler T E, Goldberg T L. Changes in physiological stress and behaviour in semi-free-ranging red-capped mangabeys (Cercocebus torquatus) following antiparasitic treatment. Proceedings of the Royal Society B-Biological Sciences. Jul. 27 2016; 283(1835)
68. Friant S, Ziegler T E, Goldberg T L. Primate reinfection with gastrointestinal parasites: behavioural and physiological predictors of parasite acquisition. Anim Behav. July 2016; 117:105-113. doi:10.1016/j.anbehav.2016.04.006
69. Walderich B, Müller L, Bracha R, Knobloch J, Burchard G D. A new method for isolation and differentiation of native Entamoeba histolytica and E. dispar cysts from fecal samples. Parasitol Res. 1997; 83(7):719-21. doi:10.1007/s004360050326
70. Caporaso J G, Kuczynski J, Stombaugh J, et al. QIIME allows analysis of high-throughput community sequencing data. Nat Methods. May 2010; 7(5):335-6. doi:10.1038/nmeth.f.303
71. Callahan B J, McMurdie P J, Rosen M J, Han A W, Johnson A J, Holmes S P. DADA2: High-resolution sample inference from Illumina amplicon data. Nat Methods. July 2016; 13(7):581-3. doi:10.1038/nmeth.3869
72. Morgulis A, Coulouris G, Raytselis Y, Madden T L, Agarwala R, Schaffer A A. Database indexing for production MegaBLAST searches. Bioinformatics. Aug. 15 2008; 24(16):1757-64. doi:10.1093/bioinformatics/btn322

### Example 2

In the following Example, the inventors describe the generation of additional reverse primers that can be used in their method for detecting eukaryotic endosymbionts (i.e., VESPA).

We originally designed four forward primers (i.e., 9F, 13F, 29F, and 2-2bF) and one reverse primer (i.e., 21b8R; TCAATTYCTTNAASTTTC (SEQ ID NO: 5)) for PCR amplification of the V4 region of the 18S gene. However, our forward primers have a higher melting temperature than the initial 21b8R reverse primer. Thus, to increase its PCR compatibility with the forward primers, the 21b8R primer was lengthened to increase its melting temperature. Specifically, we designed three additional reverse primers comprising the same core sequence of 18 nucleotides as the 21b8R primer but with 4, 5, or 6 additional nucleotides added to the 3′ end. These additional reverse primers are thus referred to as 21b8R+4 (TCCGTCAATTYCTTNAASTTTC; SEQ ID NO: 6), 21b8R+5 (TTCCGTCAATTYCTTNAASTTTC; SEQ ID NO: 7), and 21b8R+6 (CTTCCGTCAATTYCTTNAASTTTC; SEQ ID NO: 8). Note: The N shown in bold in SEQ ID NOs: 5-8 may be I, A, T, C, or G. While the reverse primers tested in this example contained an I (5-deoxyinosine) at this position, we achieved similar results with 1:1:1:1 mixtures of reverse primer variants comprising an A, T, C, or G at this position.

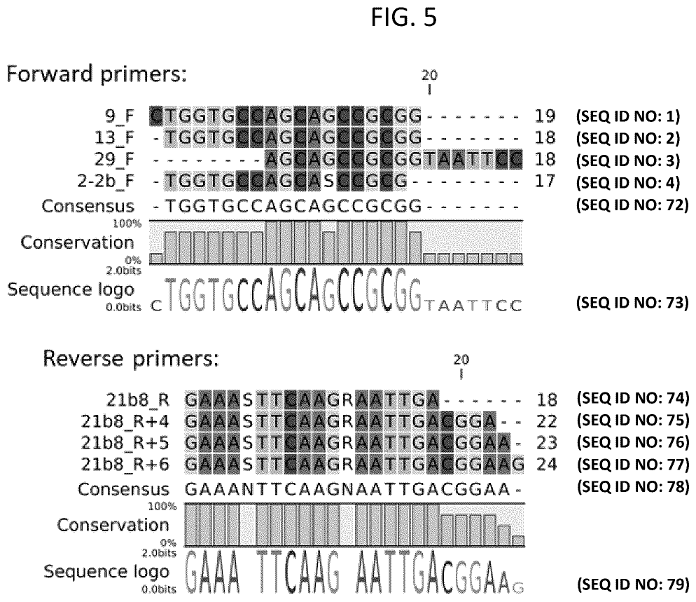

Amongst the forward and reverse primers, each have slightly different specificities based on in silico analysis (FIG. 5). The four forward primers comprise overlapping but distinct sequences, and one of the four primers comprises a single degenerate base.

In 32 published studies in which amplicon sequencing was used to identify parasites, 14 different PCR enzymes were used for the initial amplification. In preliminary tests using two different polymerases, the results varied significantly depending on the polymerase used. Thus, we next set out to determine which combination of the four forward primers, four reverse primers, and PCR polymerases would produce the best results when applied to blood and fecal samples. Specifically, we wanted to know which combination resulted in the best amplification efficiency (band size on gel), read number (total reads after quality filtering), and ratio of on-target to off-target reads (% bacterial/archaeal reads vs other). To this end, we used one blood and one fecal sample as starting material for DNA extraction and amplified the resulting DNA via PCR with various combinations of the PCR primers and eight different polymerases. After PCR, half of the reaction volume was run on an agarose gel to visualize amplification products and the other half was used for library preparation and sequencing. The resulting data were quality-filtered and assigned to taxonomic groups.

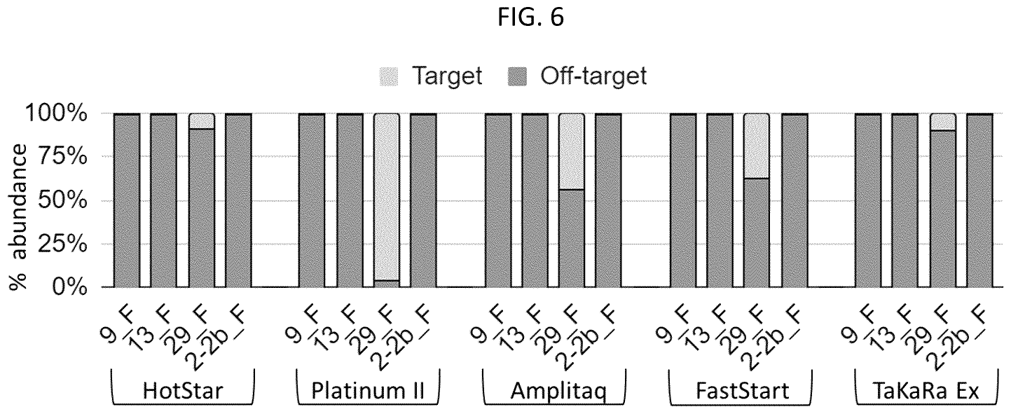

We found that amplification with all combinations of the primers and with 6 of the 8 tested polymerases resulted in consistent PCR bands and those products were used for library preparation and sequencing. For blood samples, all primer combinations and 5 of the 6 polymerases gave comparable results. For fecal samples, 1 forward primer (29F) and 1 polymerase (Platinum II HotStart) were clearly superior in terms of high read number and low off-target read abundance (FIG. 6). The +4 and +5 reverse primers performed equally well with this sample. However, we determined that the +4 reverse primer picks up slightly more protozoan parasites by performing an in silico analysis. Thus, the use of the Platinum™ II Taq Hot-Start DNA Polymerase (Invitrogen) and the primer combination 29F (SEQ ID NO: 3)/21b8R+4 (SEQ ID NO: 6) may be best suited for a universal protocol that is compatible across sample types. Based on these results, the reverse primer 21b8R+4 was selected to perform the experiments described in Example 1.

### Example 3

In the following example, the inventors describe a method for reducing host signal for sequencing-based detection of eukaryotic endosymbionts using CRISPR-Cas9 digestion.

### Introduction:

Metagenomic barcoding (metabarcoding) provides a high throughput alternative to traditional methods for reconstructing communities of host-associated organisms (1). Substantial progress has been made in methods for metabarcoding bacteria and archaea (i.e., the “microbiome”) (2) and fungi (i.e., the “mycobiome”) (3), but similar progress has lagged for eukaryotic endosymbionts (defined here as all non-fungal eukaryotes residing within vertebrate hosts, spanning the continuum of parasites to commensals and including micro- and macro-organisms) (4). One critical reason for this lag is that eukaryotic endosymbionts share highly similar DNA sequences with their eukaryotic hosts but usually at much lower concentration, leading to host signal interference (5, 6). Polymerase chain reaction (PCR) primers designed to broadly recognize eukaryotic endosymbionts (especially metazoans, such as helminths) also often bind to and amplify host DNA (i.e., non-specific, or off-target amplification) (7, 8). Primers that recognize both host and target sequences generally detect only 10⁻³ ng parasite DNA for every ng host DNA present (9). For example, spleen tissue from mice experimentally infected via tail vein injection with Leishmania donovoni harbored an average of 200 promastigotes per 0.2 mg spleen tissue, resulting in an average ng parasite DNA: ng host DNA ratio of 10⁻⁵ (10, 11). One “brute force” solution to this problem is ultra-deep sequencing—in other words, sequencing amplicons to great enough depth to compensate for host signal overabundance—but this approach is inefficient, costly, and biased against detecting low-abundance organisms (8, 12). Using metabarcoding to reconstruct eukaryotic endosymbiont assemblages from feces is commonplace, but feces is so dominated by bacterial DNA that it can also interfere with detection of eukaryotes, even using primers that appear to be eukaryote-specific (13, 14).

A reliable and efficient eukaryotic endosymbiont metabarcoding method should include a host-blocking element to enrich resulting sequences for eukaryotic endosymbiont reads in any sample type with high host DNA content (15). We refer to this process as “host signal reduction” (HSR). Published HSR methods, including restriction enzyme digestion (16), peptide nucleic acid (PNA) clamps (17), blocking oligonucleotides (18), and nested blocking primers (19). Each of these methods have advantages and disadvantages. The restriction enzyme approach, in which primers are designed such that only host amplicons contain a restriction enzyme recognition site allowing for selective cleavage of off-target amplicons prior to sequencing (20), is effective, but suitable restriction sites with flanking PCR primer sites are rare or sometimes non-existent. Selective inhibition of off-target amplification during PCR is the most commonly published host signal reduction technique (21) and can be achieved using PNA clamps or various blocking oligonucleotides (22, 23). Such methods have been used in published eukaryotic endosymbiont metabarcoding studies (24-26), but efficacy can be low, particularly in samples with high host biomass (5). Nested blocking primers were recently published for plant systems (19) but have yet to be adapted for eukaryotic endosymbiont metabarcoding and may suffer the same drawbacks as PNA clamps and blocking oligos.

CRISPR-Cas9 (CC9) mediated removal of highly abundant off-target nucleic acids is regularly used in other sequencing-based approaches, such as chromatin structure studies (27), cancer screening (28), and plant microbiome profiling (29). CC9 is a promising HSR method for eukaryotic endosymbiont metabarcoding because CRISPR-Cas9 nuclease activity is highly specific (30), reagents are readily available and relatively inexpensive, and the reaction components are modular such that different hosts or read types (e.g., dietary or environmental sequences) can be eliminated depending on experimental requirements. To our knowledge, however, CC9 has not been applied to HSR in the context of eukaryotic endosymbiont metabarcoding.

Here we assess the most commonly published HSR protocol for eukaryotic endosymbiont metabarcoding, the use of a PNA blocker, and demonstrate the need for a more effective approach. We design such a method based on a recombinant Streptococcus pyogenes CC9 system, in which vertebrate sequences are selectively targeted for cleavage and removal by host-specific guide RNAs (gRNAs) while leaving amplicons of interest intact for sequencing and analysis. Using in silico analyses, in vitro digests, and samples from experimentally infected animals, we show that our method is more effective than published HSR methods across various sample types. Finally, we compare the efficacy of eukaryotic endosymbiont metabarcoding for detection of known parasite infections and show that CC9 host signal reduction is necessary to detect hemoparasites in blood samples from naturally infected hosts.

### Results:

### High Host Read Abundance in 18S V4 Metabarcoding Data Using a PNA Clamp

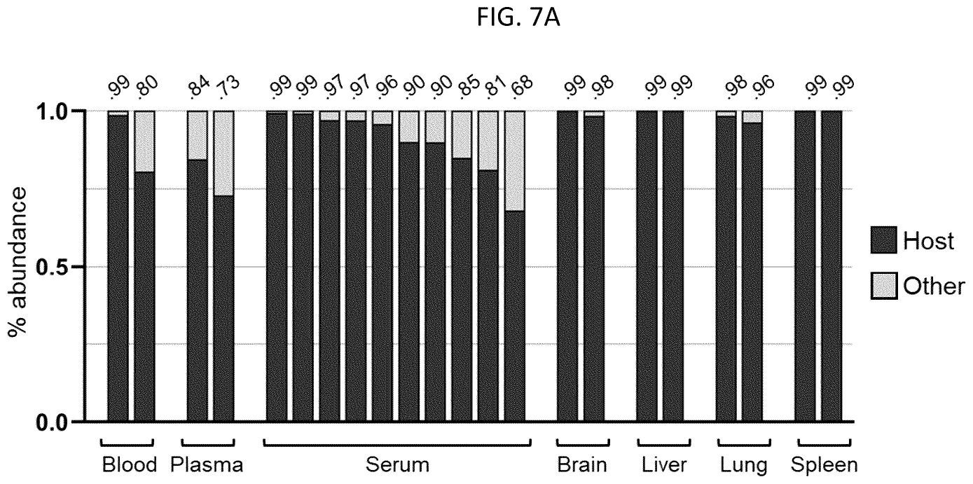

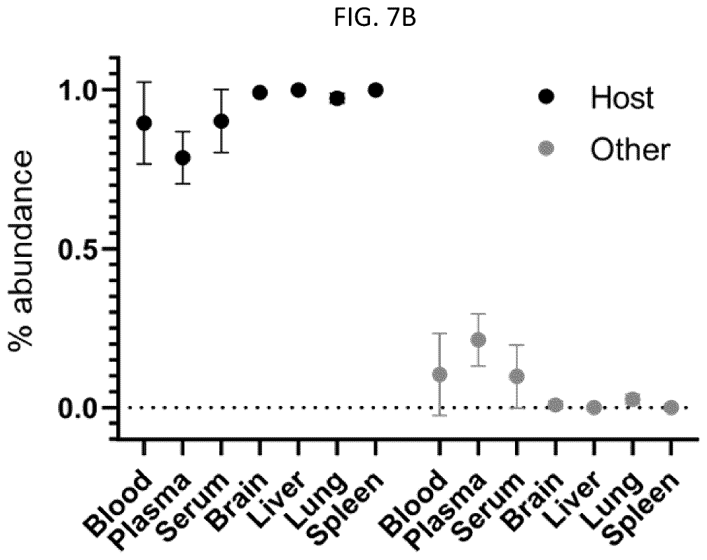

18S V4 metabarcoding (24, 42) using DNA extracted from chimpanzee samples as input (n=28) and including the mammal-blocking PNA clamp in every amplification (24) yielded a wide range of host signal relative abundances (FIG. 7A). The percent abundance of host reads obtained was low in fecal samples (overall mean <1%) but high in all other sample types tested, including blood, plasma, serum, brain, liver, lung, spleen (overall mean=93.5%; Table 12). Of non-fecal samples, plasma samples contained the lowest relative abundance of host reads (mean=78.6%) and spleen samples contained the highest (mean=99.9%; FIG. 7B).

TABLE 12
Descriptive statistics of read data obtained from 18S
V4 metabarcoding with PNA mammal blocker applied to
nonhuman primate fecal, blood, and tissue samples

% host reads after quality filtering

| Sample | n | Mean | SEM | Min | Max |
|---|---|---|---|---|---|
| Feces | 6 | 0.0078 | 0.0042 | 0.0000 | 0.0290 |
| Blood | 2 | 0.8954 | 0.0644 | 0.8044 | 0.9864 |
| Plasma | 2 | 0.7865 | 0.0410 | 0.7286 | 0.8445 |
| Serum | 10 | 0.9018 | 0.0297 | 0.6806 | 0.9928 |
| Brain | 2 | 0.9912 | 0.0056 | 0.9832 | 0.9992 |
| Liver | 2 | 0.9998 | 0.0000 | 0.9998 | 0.9999 |
| Lung | 2 | 0.9732 | 0.0074 | 0.9627 | 0.9837 |
| Spleen | 2 | 0.9999 | 0.0000 | 0.9999 | 0.9999 |

### Guide RNA Design for Universal Eukaryotic Endosymbiont Enrichment

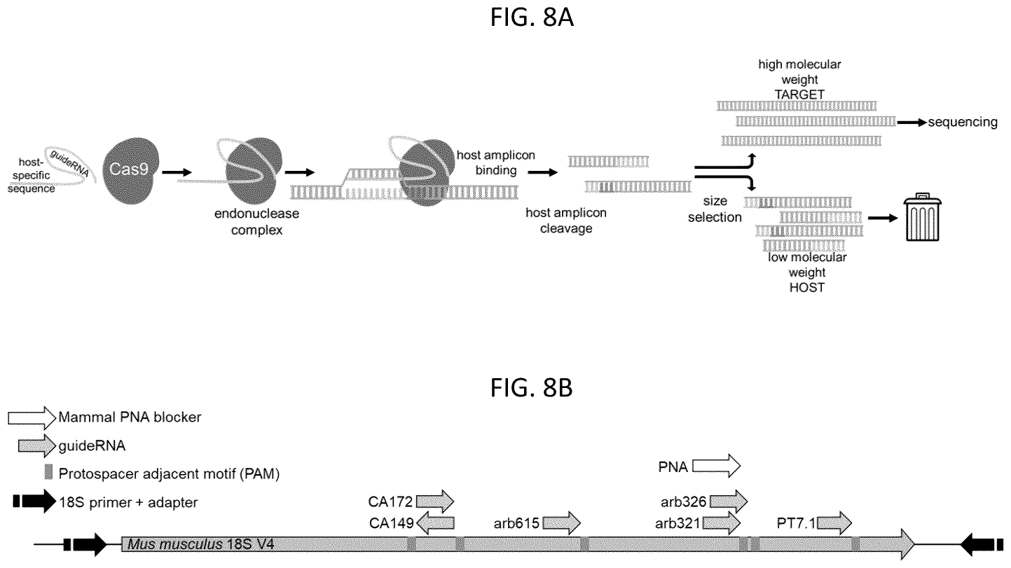

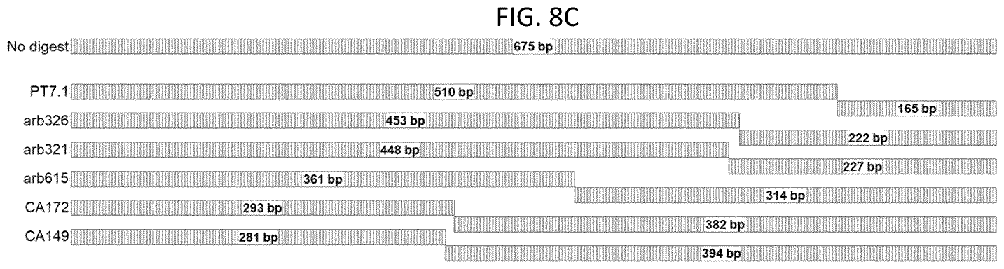

We designed six candidate vertebrate host-specific gRNAs targeting 18S V4 (FIG. 8A), including one fortuitously identical to the published 18S V4 mammal-blocking PNA oligo used above (arb321; Table 13) (24). Host DNA sequences targeted by the gRNAs all include a protospacer adjacent motif (PAM) “NGG” required by the Streptococcus pyogenes Cas9 enzyme. Target sites are located centrally in 18S V4 (FIG. 8B) such that the digestion products can be differentiated from uncleaved amplicons based on size (FIG. 8C).

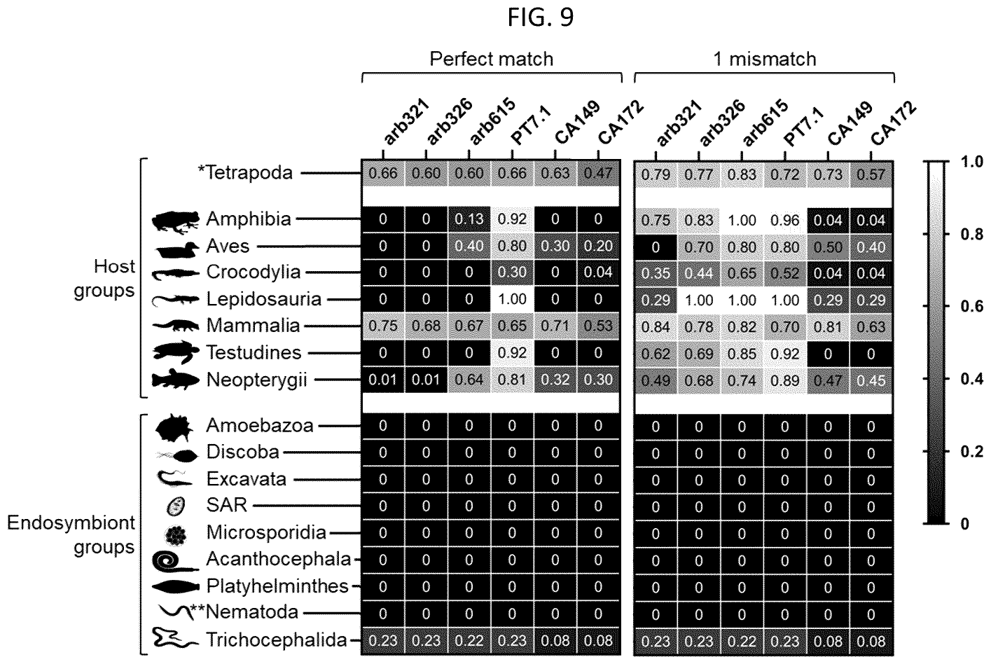

Using in silico hybridization to the SILVA 138 RefNR database (35) we found all six candidates to have similar mammalian complementarity (FIG. 9), with each hybridizing to 50% or more of mammalian sequences (mean=66.4%) with no mismatches and 60% or more when allowing for a single mismatch (mean=76.4%). gRNAs arb321 and arb326 were effective for mammalian hosts, but several gRNAs additionally recognized non-mammalian vertebrate groups, making them useful for a wider variety of hosts: arb615, CA149, and CA172 recognized mammal, bird, and fish sequences, while PT7.1 recognized all vertebrates (Table 13). All six gRNA oligos failed to hybridize to any parasite/endosymbiont group, with the sole exception of Trichinella pseudospiralis (mean=17.8%; FIG. 9) due to high 18S sequence similarity between Trichinella and mammals (mean=45.5% DNA identity for all gRNA target regions combined in Trichinella pseudospiralis AY851258; Table 14).

TABLE 13
gRNA sequences and characteristics

| | | SEQ | Orien- | PAM | GC | Seed | |
|---|---|---|---|---|---|---|---|
| ID | Target/gRNA Seq | ID NO: | tation | Seq | % | seq | Host specificity** |
| arb321* | AACTGAGGCCATGATTAAGA* | 9 | sense | GGG | 45 | TTAAGA | Mammals |
| arb326 | AGGCCATGATTAAGAGGGA | 10 | sense | CGG | 40 | GAGGGA | Mammals |
| arb615 | GCAGCTAGGAATAATGGAAT | 11 | sense | AGG | 55 | TGGAAT | Mammals, Birds, Fish |
| PT7.1 | ATTCTTGGACCGGCGCAAGA | 12 | sense | CGG | 40 | GCAAGA | Vertebrates |
| CA149 | CTCAGCTAAGAGCATCGAGG | 13 | antisense | GGG | 60 | ATCGAGG | Mammals, Birds, Fish |
| CA172 | TCTTAGCTGAGTGTCCCGCG | 14 | sense | GGG | 55 | CCCGCG | Mammals, Birds, Fish |

gRNA, guide RNA; seq, sequence; PAM, protospacer adjacent motif;
*sequence identical to V4 mammal blocking PNA oligo used in Mann et al. 2020;
**specificity to host groups determined by SILVA TestProbe in silico hybridization data.

TABLE 14
18S V4 region comparison and percent DNA identity between gRNAs, mouse, and Trichinella
pseudospiralis sequences

| | | | Mouse | Trichinella | TP% |
|---|---|---|---|---|---|
| gRNA | Target/gRNA Seq | Mouse Seq NR 003278* | % ID | Seq AY851258** | ID |
| arb321 | AACTGAGGCCATGATTAAGA | AACTGAGGCCATGATTAAGA | 100% | ACCGGAGATAAGTATTGAAA | 55% |
| | (SEQ ID NO: 9) | (SEQ ID NO: 9) | | (SEQ ID NO: 68) | |
| arb326 | AGGCCATGATTAAGAGGGA | AGGCCATGATTAAGAGGGA | 100% | AGATAAGTATTGAAAGGAA | 58% |
| | (SEQ ID NO: 10) | (SEQ ID NO: 10) | | (SEQ ID NO: 69) | |
| arb615 | GCAGCTAGGAATAATGGAAT | GCAGCTAGGAATAATGGAAT | 100% | GGTGCATGGAATAATAGAAT | 75% |
| | (SEQ ID NO: 11) | (SEQ ID NO: 11) | | (SEQ ID NO: 70) | |
| PT7.1 | ATTCTTGGACCGGCGCAAGA | ATTCTTGGACCGGCGCAAGA | 100% | ATTCTTGGATCGCAGCAAGA | 85% |
| | (SEQ ID NO: 12) | (SEQ ID NO: 12) | | (SEQ ID NO: 71) | |
| CA149 | CTCAGCTAAGAGCATCGAGG | CTCAGCTAAGAGCATCGAGG | 100% | NA | 0% |
| | (SEQ ID NO: 13) | (SEQ ID NO: 13) | | | |
| CA172 | TCTTAGCTGAGTGTCCCGCG | TCTTAGCTGAGTGTCCCGCG | 100% | NA | 0% |
| | (SEQ ID NO: 14) | (SEQ ID NO: 14) | | | |
| | | Mean | 100% | Mean | 45.5% |

### CRISPR-Cas9 In Vitro Digestion Selectively Cleaves Target Organisms

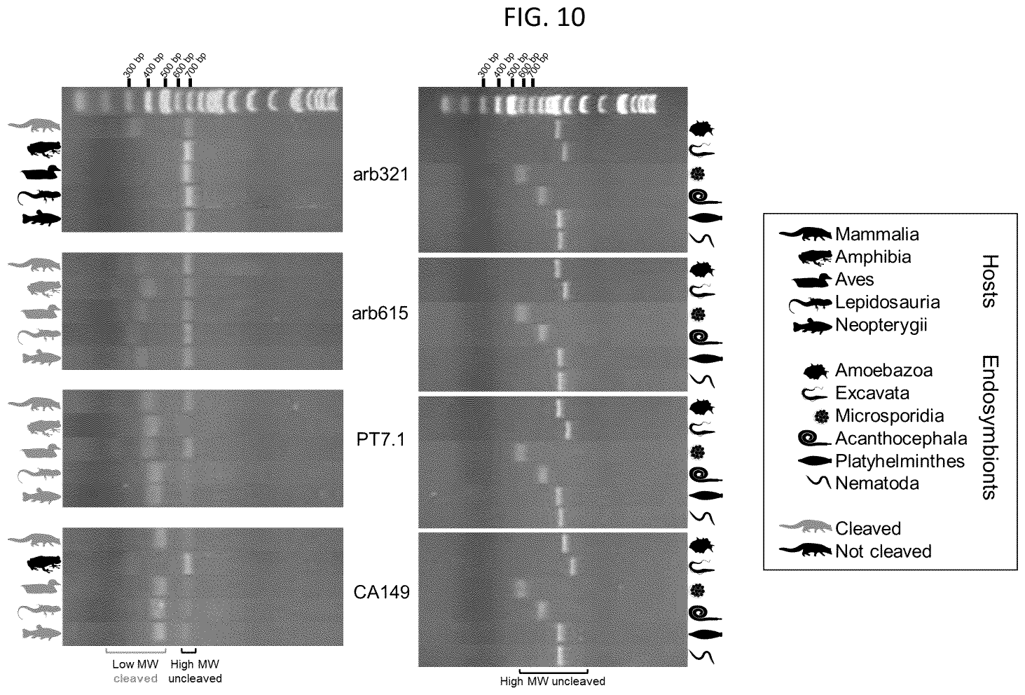

In vitro digests of 18S V4 amplicons from single representative vertebrate hosts and eukaryotic endosymbionts corresponded to SILVA TestProbe predicted coverages (FIG. 9) and fragment sizes (FIG. 8B). For example, CC9 digestion with the “mammal” arb321 gRNA resulted in cleavage of mammal samples, but not amphibian, reptile, bird, or fish samples, whereas digestion with the “vertebrate” PT7.1 gRNA resulted in cleavage of all 5 host samples including mammal, amphibian, reptile, bird, and fish (FIG. 10, left panel). All eukaryotic endosymbiont amplicons, including protozoans (n=2), microsporidians (n=1), and helminths (n=3) were unaffected by CC9 digestion using any gRNA (FIG. 10, right panel).

### Evaluating Host Signal Reduction Methods

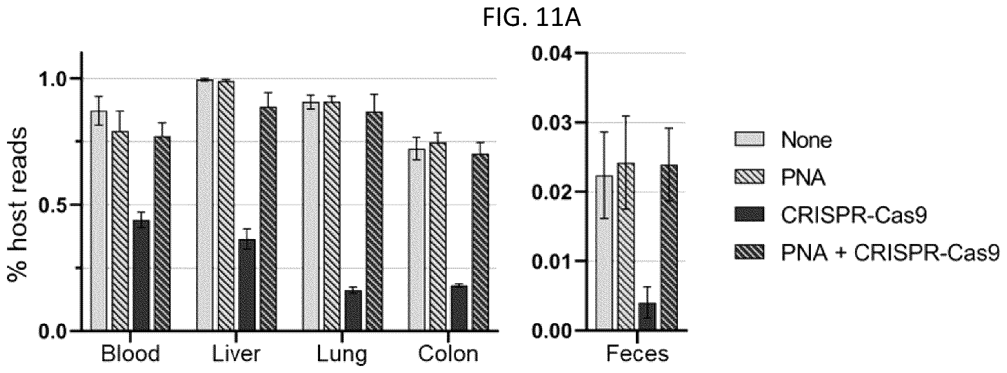

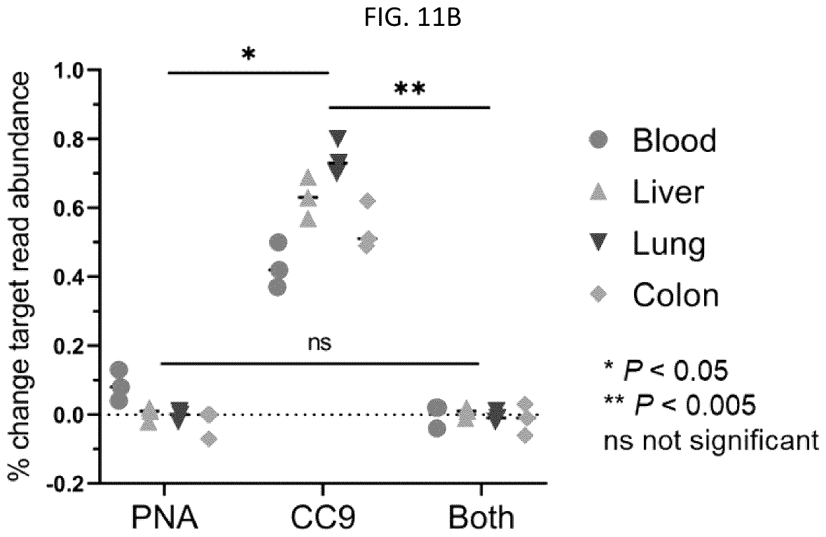

18S V4 metabarcoding using DNA extracted from chimpanzee samples as input (n=15) with PNA blocker, CC9 digest, both PNA and digest, and no host signal reduction demonstrated CC9 digest to be the most effective method for enriching target read abundance for all sample types (blood, liver, lung, colon, and fecal samples; FIG. 11A; Table 15). Fecal samples yielded consistently low levels of host reads and were therefore not analyzed further. In tissue samples (blood, liver, lung, and colon), the overall percentage change in target (non-host) reads compared to no treatment control was significantly higher for CC9 treatment (mean 58.7% increase in target reads, SEM 3.6%, range 37.2%-79.9%) compared to PNA (mean 1.5%, SEM 1.3%, range −7.1%-12.6%; paired t-test: t=6.94, df=3, P=0.0061) or combination treatment (mean −0.2%, SEM 0.7%, range −5.6%-2.9%; paired t-test: t=8.89, df=3, P=0.0030; FIG. 11B).

TABLE 15
Descriptive statistics of read data obtained from 18S V4 metabarcoding
with CRISPR-Cas9 digest, PNA mammal blocker, both CRISPR-
Cas9 digest and PNA mammal blocker, or no treatment applied
to nonhuman primate blood and tissue samples

| | | | % host reads after quality filtering | | | |
|---|---|---|---|---|---|---|
| Sample | Treatment | n | Mean | SEM | Min | Max |
| Blood | None | 3 | 0.8719 | 0.0470 | 0.8044 | 0.9864 |
| | PNA | 3 | 0.7918 | 0.0644 | 0.6983 | 0.9485 |
| | CC9 | 3 | 0.4399 | 0.0249 | 0.3819 | 0.4851 |
| | Both | 3 | 0.8714 | 0.0413 | 0.8006 | 0.9693 |
| Liver | None | 3 | 0.9946 | 0.0043 | 0.9841 | 0.9999 |
| | PNA | 3 | 0.9894 | 0.0044 | 0.9806 | 0.9992 |
| | CC9 | 3 | 0.3643 | 0.0327 | 0.2924 | 0.4306 |
| | Both | 3 | 0.9846 | 0.0032 | 0.9775 | 0.9909 |
| Lung | None | 3 | 0.9067 | 0.0230 | 0.8736 | 0.9627 |
| | PNA | 3 | 0.9081 | 0.0182 | 0.8746 | 0.9504 |
| | CC9 | 3 | 0.1620 | 0.0096 | 0.1406 | 0.1811 |
| | Both | 3 | 0.9116 | 0.0246 | 0.8669 | 0.9689 |
| Colon | None | 3 | 0.7223 | 0.0363 | 0.6752 | 0.8112 |
| | PNA | 3 | 0.7463 | 0.0314 | 0.6769 | 0.8099 |
| | CC9 | 3 | 0.1805 | 0.0052 | 0.1688 | 0.1907 |
| | Both | 3 | 0.7353 | 0.0210 | 0.6926 | 0.7814 |
| Fecal | None | 3 | 0.0224 | 0.0051 | 0.0143 | 0.0347 |
| | PNA | 3 | 0.0242 | 0.0055 | 0.0166 | 0.0376 |
| | CC9 | 3 | 0.0040 | 0.0018 | 0.0007 | 0.0083 |
| | Both | 3 | 0.0239 | 0.0043 | 0.0162 | 0.0339 |

### Optimization of CRISPR-Cas9 Digest

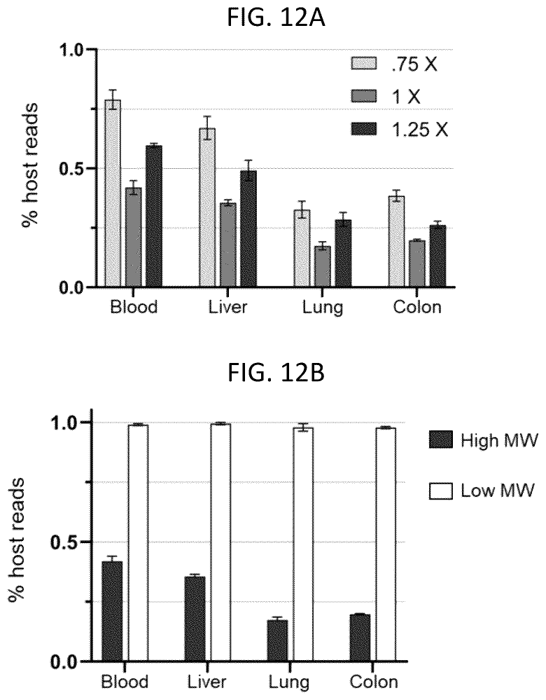

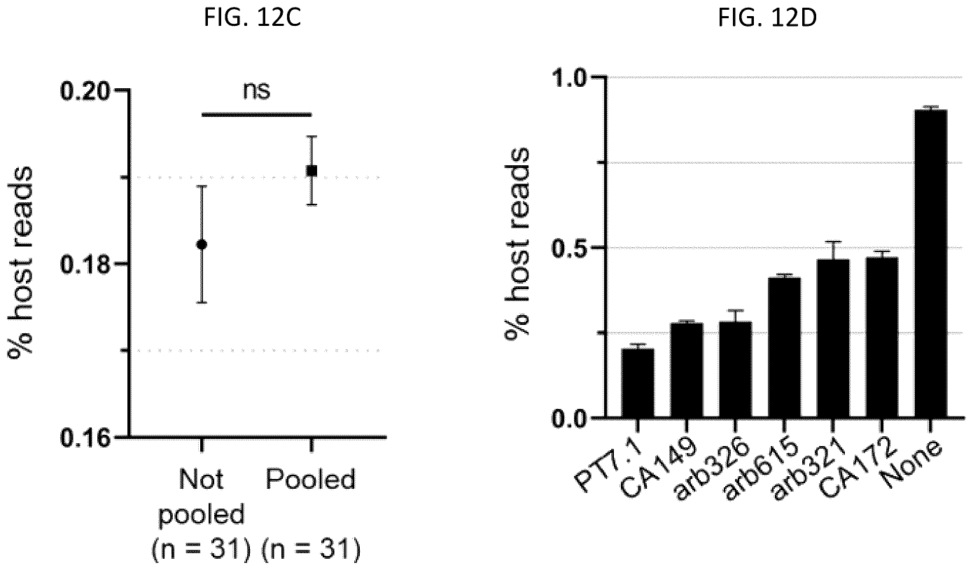

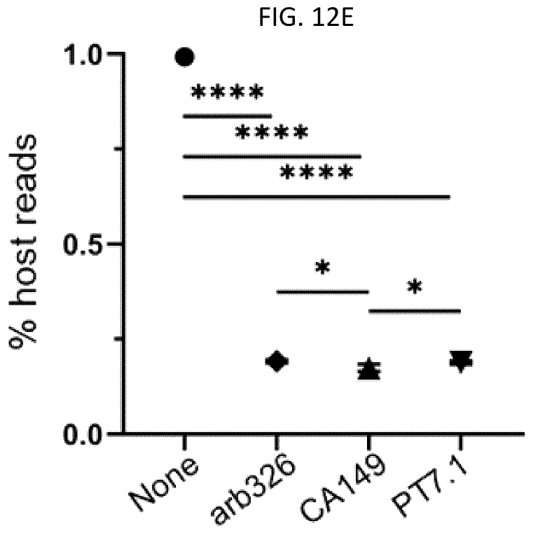

We optimized parameters of the CC9 digest by varying at the ratio of ribonucleoprotein complex to target DNA PAM sequence and found that a ratio of 1:1 was most effective at lowering host signal (FIG. 12A). To confirm the identity of the low molecular weight (MW) bands resulting from CC9 digest of mixed samples (containing both host and parasite DNA), we compared host read abundance in the higher- and lower-MW bands to show that the cleaved products are indeed of host origin (FIG. 12B). We also evaluated the application of the CC9 digest before and after indexing PCR. There was no significant difference in digest efficiency for CC9 treatment applied to each individual amplicon prior to library preparation compared to CC9 applied to a library pool (paired t-test: t=0.38, df=30, P=0.18; FIG. 12C). Because application of the digest after indexing is simpler and cheaper, we used this variation of the HSR protocol for all subsequent metabarcoding experiments.

18S V4 metabarcoding using a panel of all six newly designed gRNAs demonstrated all gRNAs to reduce host signal compared to mock-treated controls, with vertebrate gRNA PT7.1 having the lowest abundance and mammal gRNA arb321 having the highest (FIG. 12D; Table 16). Further testing using the three top-performing gRNAs (arb326, CA149, and PT7.1) showed that digestion with any of the three gRNAs significantly reduced host reads compared to no-treatment controls (arb326 compared to none, paired t-test: t=282.2, df=30, P<0.0001; CA149 compared to none, paired t-test: t=123.6, df=30, P<0.0001; PT7.1 compared to non, paired t-test: t=370.3, df=30, P<0.001). There was also a small, but significant difference in signal reduction among the three gRNAs, with CA149 being most effective (CA149 compared to arb326, paired t-test: t=2.10, df=30, P=0.049; CA149 compared to PT7.1, paired t-test: t=2.52, df=30, P=0.021; FIG. 12E; Table 16).

TABLE 16
Descriptive statistics of read data obtained from 18S
V4 metabarcoding with CRISPR-Cas9 digest or no treatment
applied to nonhuman primate blood samples

% host reads after quality filtering

| gRNA | n | Mean | SEM | Min | Max |
|---|---|---|---|---|---|
| PT7.1 | 3 | 0.2040 | 0.0095 | 0.1905 | 0.2174 |
| CA149 | 3 | 0.2792 | 0.0046 | 0.2726 | 0.2857 |
| arb326 | 3 | 0.2829 | 0.0233 | 0.2500 | 0.3158 |
| arb615 | 3 | 0.4129 | 0.0059 | 0.4045 | 0.4212 |
| arb321 | 3 | 0.4663 | 0.0366 | 0.4146 | 0.5180 |
| CA172 | 3 | 0.4718 | 0.0122 | 0.4545 | 0.4891 |
| NONE | 3 | 0.9037 | 0.0070 | 0.8937 | 0.9136 |
| Arb326 | 31 | 0.1917 | 0.0038 | 0.0974 | 0.2433 |
| CA149 | 31 | 0.1744 | 0.0080 | 0.0587 | 0.2211 |
| PT7.1 | 31 | 0.1886 | 0.0043 | 0.1027 | 0.2568 |
| NONE | 31 | 0.9926 | 0.0033 | 0.9097 | 1.0000 |

### CRISPR-Cas9 Digest Validation Using Known Parasite Infections of Mammals

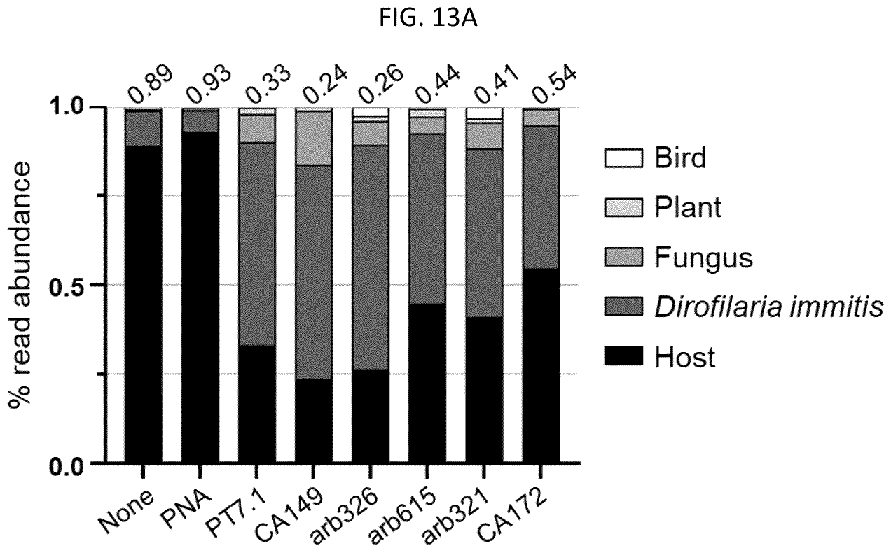

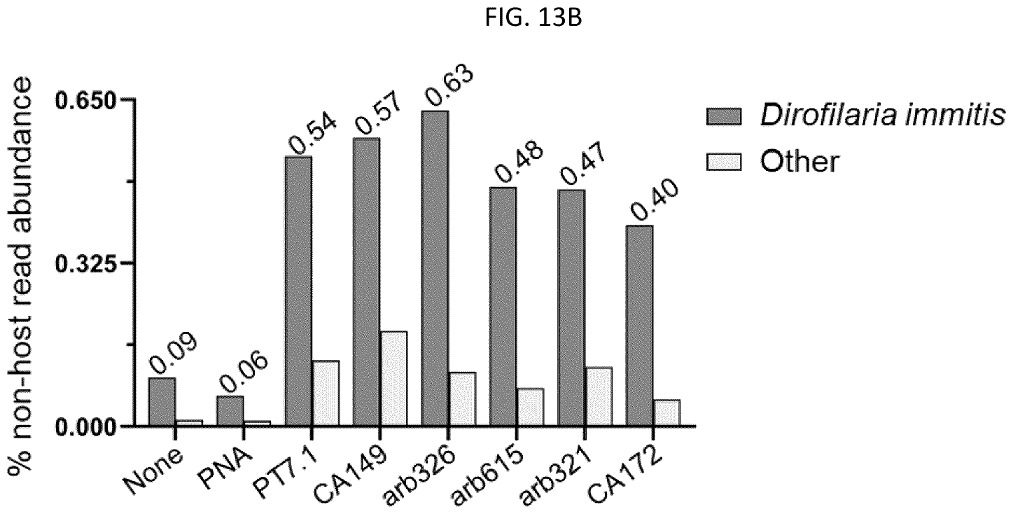

Dirofilaria immitis in experimentally infected dogs. 18S V4 metabarcoding of experimentally infected dog blood samples containing Dirofilaria immitis microfilariae (mean 57.8 microfilariae per 20 μl whole blood) demonstrated CC9 digestion to be more effective at host signal reduction than PNA blocking oligo or mock treatment (FIG. 13A). Specifically, CC9-digested samples yielded a higher abundance of Dirofilaria immitis reads (mean of 6 gRNAs=37.24%, SEM=4.38%, range: 23.66%-54.59%) than did PNA blocking oligo treatment (92.77%) or mock control (88.96%). Intriguingly, CC9-digested samples also recovered reads from fungi and dietary items that were not detected by the other methods (FIG. 13B; Table 17).

TABLE 17
Descriptive statistics of read data obtained from 18S V4 metabarcoding
with CRISPR-Cas9 digest or no treatment applied to dog blood
samples with known Dirofilaria immitis infection

| | % reads after quality filtering | | | | |
|---|---|---|---|---|---|
| | | Dirofilaria | | | |
| Treatment | Host | immitis | Fungi | Plant | Bird |
| None | 0.8896 | 0.0977 | 0.0037 | 0.0074 | 0.0016 |
| PNA | 0.9277 | 0.0608 | 0.0010 | 0.0065 | 0.0039 |
| PT7.1 | 0.3308 | 0.5684 | 0.0780 | 0.0193 | 0.0036 |
| CA149 | 0.2366 | 0.5999 | 0.1505 | 0.0128 | 0.0002 |
| arb326 | 0.2634 | 0.6281 | 0.0664 | 0.0155 | 0.0266 |
| arb615 | 0.4473 | 0.4763 | 0.0461 | 0.0233 | 0.0070 |
| arb321 | 0.4104 | 0.4716 | 0.0725 | 0.0114 | 0.0339 |
| CA172 | 0.5460 | 0.4006 | 0.0455 | 0.0030 | 0.0049 |
| CC9 Mean | 0.3724 | 0.5241 | 0.0765 | 0.0142 | 0.0127 |

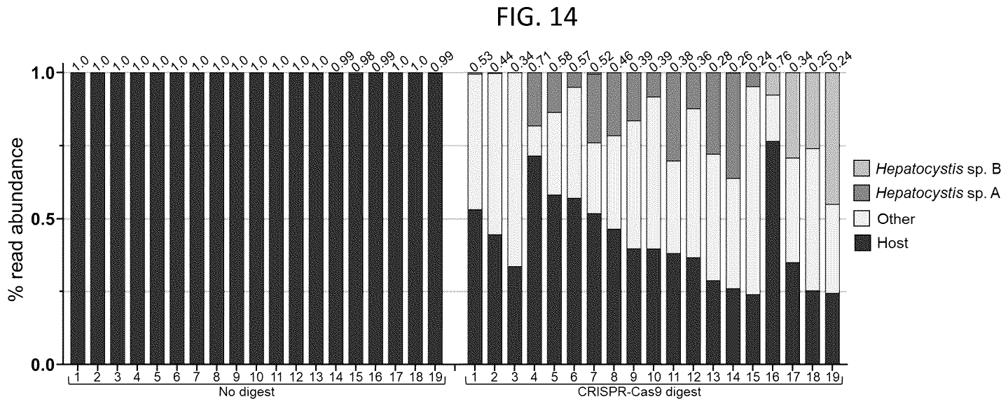

Hepatocystis in naturally infected red colobus. Data from wild red colobus blood samples demonstrated that, in untreated libraries, almost all reads were of host origin (mean=99.9%) and no hemoparasites were detected. By contrast, CC9 treated libraries from the same samples had, on average, only 42.6% host reads, and hemoparasites were detected in 17 of 19 samples (FIG. 14; Table 18). These findings mirrored previous results from Hepatocystis-specific PCR of these same samples (32), in which the same two species/lineages of Hepatocystis were detected: species A in 13 of the 17 infected samples and species B in 5 of the 17 infected samples (Table 19). Two samples were positive by metabarcoding that were negative by PCR. Percent agreement was low between PCR and metabarcoding without HSR treatment (Cohen's Kappa test: κ=0.0, 95% CI from 0.0 to 0.0) and high between PCR and metabarcoding with CC9 digest (Cohen's Kappa test: κ=0.855, 95% CI from 0.581 to 1.000). Overall application of CC9 digest increased agreement with PCR 6-fold compared to no treatment (Table 20).

TABLE 18
Descriptive statistics of read data obtained from 18S V4 metabarcoding with
CRISPR-Cas9 digest or no treatment applied to red colobus blood samples

| | | | | | | | | | No |
|---|---|---|---|---|---|---|---|---|---|
| | No treatment | | | | CRISPR-Cas9 digest | | | | treatment |
| | | | % | % | | | Hepatocystis | Hepatocystis | vs CC9 |
| | | % | Hepatocystis | Hepatocystis | | | sp. A | sp. B | % change |
| | % host | other | sp. A | sp. B | % host | % other | % | % | host reads |
| 1 | 1.0000 | 0.0000 | 0.0000 | 0.0000 | 0.5322 | 0.4634 | 0.0022 | 0.0000 | 0.4678 |
| 2 | 1.0000 | 0.0000 | 0.0000 | 0.0000 | 0.4455 | 0.5522 | 0.0000 | 0.0000 | 0.5545 |
| 3 | 1.0000 | 0.0000 | 0.0000 | 0.0000 | 0.3363 | 0.6637 | 0.0000 | 0.0000 | 0.6637 |
| 4 | 1.0000 | 0.0000 | 0.0000 | 0.0000 | 0.7154 | 0.1028 | 0.1818 | 0.0000 | 0.2846 |
| 5 | 1.0000 | 0.0000 | 0.0000 | 0.0000 | 0.5820 | 0.2827 | 0.1353 | 0.0000 | 0.4180 |
| 6 | 1.0000 | 0.0000 | 0.0000 | 0.0000 | 0.5712 | 0.3793 | 0.0494 | 0.0000 | 0.4288 |
| 7 | 1.0000 | 0.0000 | 0.0000 | 0.0000 | 0.5185 | 0.2420 | 0.2349 | 0.0046 | 0.4815 |
| 8 | 0.9999 | 0.0001 | 0.0000 | 0.0000 | 0.4639 | 0.3209 | 0.2152 | 0.0000 | 0.5360 |
| 9 | 0.9999 | 0.0001 | 0.0000 | 0.0000 | 0.3971 | 0.4385 | 0.1643 | 0.0000 | 0.6028 |
| 10 | 0.9996 | 0.0004 | 0.0000 | 0.0000 | 0.3966 | 0.5205 | 0.0829 | 0.0000 | 0.6030 |
| 11 | 0.9994 | 0.0006 | 0.0000 | 0.0000 | 0.3810 | 0.3174 | 0.3017 | 0.0000 | 0.6184 |
| 12 | 0.9994 | 0.0006 | 0.0000 | 0.0000 | 0.3668 | 0.5100 | 0.1232 | 0.0000 | 0.6326 |
| 13 | 0.9985 | 0.0015 | 0.0000 | 0.0000 | 0.2880 | 0.4337 | 0.2783 | 0.0000 | 0.7105 |
| 14 | 0.9981 | 0.0019 | 0.0000 | 0.0000 | 0.2604 | 0.3788 | 0.3599 | 0.0000 | 0.7377 |
| 15 | 0.9966 | 0.0034 | 0.0000 | 0.0000 | 0.2402 | 0.7125 | 0.0473 | 0.0000 | 0.7564 |
| 16 | 1.0000 | 0.0000 | 0.0000 | 0.0000 | 0.7654 | 0.1586 | 0.0000 | 0.0759 | 0.2346 |
| 17 | 1.0000 | 0.0000 | 0.0000 | 0.0000 | 0.3494 | 0.3589 | 0.0000 | 0.2912 | 0.6506 |
| 18 | 0.9998 | 0.0002 | 0.0000 | 0.0000 | 0.2534 | 0.4870 | 0.0000 | 0.2595 | 0.7464 |
| 19 | 0.9988 | 0.0012 | 0.0000 | 0.0000 | 0.2445 | 0.3058 | 0.0000 | 0.4497 | 0.7543 |

TABLE 19
Hepatocystis detection by PCR versus metabarcoding
with and without CRISPR-Cas9 digestion

| | PCR | | Metabarcoding, no treatment | | Metabarcoding, CC9 digest | |
|---|---|---|---|---|---|---|
| | Positive/Negative | | % reads post quality filtering | | % reads post quality filtering | |
| | Hepatocystis | Hepatocystis | Hepatocystis | Hepatocystis | Hepatocystis | Hepatocystis |
| ID # | sp. A | sp. B | sp. A | sp. B | sp. A | sp. B |
| 1 | Negative | Negative | 0 | 0 | 0.002 | 0 |
| 2 | Negative | Negative | 0 | 0 | 0 | 0 |
| 3 | Negative | Negative | 0 | 0 | 0 | 0 |
| 4 | Positive | Negative | 0 | 0 | 0.182 | 0 |
| 5 | Positive | Negative | 0 | 0 | 0.135 | 0 |
| 6 | Positive | Negative | 0 | 0 | 0.049 | 0 |
| 7 | Positive | Negative | 0 | 0 | 0.235 | 0.005 |
| 8 | Positive | Negative | 0 | 0 | 0.215 | 0 |
| 9 | Positive | Negative | 0 | 0 | 0.164 | 0 |
| 10 | Positive | Negative | 0 | 0 | 0.083 | 0 |
| 11 | Positive | Negative | 0 | 0 | 0.302 | 0 |
| 12 | Positive | Negative | 0 | 0 | 0.123 | 0 |
| 13 | Positive | Negative | 0 | 0 | 0.278 | 0 |
| 14 | Positive | Negative | 0 | 0 | 0.36 | 0 |
| 15 | Positive | Negative | 0 | 0 | 0.047 | 0 |
| 16 | Negative | Positive | 0 | 0 | 0 | 0.076 |
| 17 | Negative | Positive | 0 | 0 | 0 | 0.291 |
| 18 | Negative | Positive | 0 | 0 | 0 | 0.26 |
| 19 | Negative | Positive | 0 | 0 | 0 | 0.45 |

TABLE 20
Percentage agreement statistics using Cohen's Kappa test of
read data obtained from 18S V4 metabarcoding with CRISPR-Cas9
digest or no treatment applied to red colobus blood samples

| | | PCR | |
|---|---|---|---|
| | | Negative | Positive |
| Metabarcoding, | Negative | 3 | 16 |
| No HSR | Positive | 0 | 0 |
| | # observed agreements | 3 | |
| | % observed agreements | 15.79% | |
| | Kappa | 0 | |
| | SE of Kappa | 0 | |
| | 95% CI | 0 to 0 | |
| Metabarcoding, | Negative | 2 | 0 |
| CC9 digest | Positive | 1 | 16 |
| | # observed agreements | 18 | |
| | % observed agreements | 94.74% | |
| | Kappa | 0.855 | |
| | SE of Kappa | 0.14 | |
| | 95% CI | 0.581 to 1.000 | |

### Discussion:

Here we show that a newly designed method using CRISPR-Cas9 and vertebrate host-targeted guide RNAs was more effective at host signal reduction than PNA blocking or no treatment. Furthermore, in samples known from prior analyses to contain parasites, eukaryotic endosymbiont reads were rare or not detectable in samples treated with a PNA blocking primer or not treated with any HSR method. However, when the new CC9 method was applied to these same samples, the parasites were detected at high read intensities. The new CC9 method also yielded reads matching two lineages of Hepatocystis previously characterized in red colobus using genus specific PCR (32).

The utility of the CC9 HSR method depends on the specificity of gRNAs (36, 43). We attempted to maximize specificity by designing gRNAs using several complementary approaches and screening a large pool of 100 candidate oligos to identify six final gRNA sequences. We then rigorously evaluated these six oligos in silico and in laboratory experiments using gDNA from individual eukaryotic organisms and from clinical samples infected with eukaryotic parasites. The consistency of our results across these conditions strongly suggests that the CC9 method is specific, effective, and robust. We note, however, that 8%-23% of sequences from the nematode parasite Trichinella pseudospiralis were highly similar to the mammalian 18S V4 region CC9 recognition sites, reducing specificity in the case of this genus. If Trichinella is suspected, we recommend the use of gRNAs CA 149 and CA 172, which have the lowest cross-reactivity. We also recommend in silico analysis to verify host complementarity prior to choosing a particular gRNA.

A distinct advantage of our method is that it does not depend on the PCR primers used to amplify the 18S V4 region, as long as those primers flank the site of gRNA complementarity. Therefore, any amplicon including the 18S V4 region is compatible with all gRNA oligos presented here. As is described in Example 1, we recently developed a new set of eukaryotic endosymbiont metabarcoding primers that out-performs all other published primer sets in terms of taxonomic breath, on-target amplification, and unbiased reconstruction of eukaryotic communities. We have examined this primer set in conjunction with the CC9 protocol described herein, and in combination the two methods achieve a similar reduction of host signal as this study (82% less host reads compared to no treatment and 74% compared to PNA clamp in blood samples; unpublished data). Also, because 18S V4 has the highest entropy of the hypervariable regions constituting 18S (44, 45), and thus the highest taxonomic resolution, we expect our gRNA designs to stay relevant for as long as this locus remains the industry standard for eukaryotic endosymbiont metabarcoding.

Overall, we have shown that CRISPR-Cas9 digestion of amplicons reduces host signal sufficiently to allow for detection of rare eukaryotic endosymbionts and thus to increase the sensitivity and efficiency of eukaryotic endosymbiont metabarcoding. Our new method should help advance the fields of parasitology and eukaryotic community ecology, similar to how 16S prokaryote metabarcoding has facilitated the study the microbiome.

### Materials and Methods

### Sample Collection and Characterization

We used archived blood, tissue, and fecal samples from wild nonhuman primates, including western chimpanzees (Pan troglodytes verus) from Sierra Leone and red colobus (Procolobus rufomitratus) from Uganda that had been collected as part of previous studies (31, 32). Appropriate permits and approvals were obtained by each research team prior to collection and shipping of samples. Blood and tissue samples from chimpanzees had been assessed for pathogenic organisms as described in Owens, et al (31). Blood samples from red colobus had been assessed for Hepatocystis parasites as described in Thurber, et al (32). Blood from domestic dogs (Canis lupis familiaris) experimentally infected with Dirofilaria immitis strain “Missouri” was obtained via BEI resources (Catalog #NR-48907; Manassas, VA, USA), 20 μl was added to a glass slide and combined with two drops of 2% formalin, and microfilaria were enumerated using phase optics at ×10 magnification. Samples were examined in triplicate and load was expressed as number of microfilariae per 20 μl of blood averaged across the three replicates. Genomic DNA (gDNA) from single hosts and parasites were obtained from in-house sample archives retained from prior studies (Owens VESPA).

### DNA Extraction and 18S V4 Metabarcoding

Fecal samples were thawed on ice and homogenized by vortexing prior to transferring 0.2 g of homogenate to bead beating tubes for DNA extraction using the DNeasy PowerLyzer PowerSoil Kit (Qiagen, Hilden, Germany), with gDNA eluted in C6 buffer and stored at −20° C. Whole blood, serum, and plasma were thawed on ice and solid tissue samples were subsampled with a sterile 3 mm biopsy punch (Integra Life Sciences, Princeton, NJ, USA) while still frozen. gDNA was extracted from blood products and tissue samples using the Qiagen DNeasy Blood and Tissue kit following manufacturer's instructions, eluted in buffer AE, and stored at −20° C.

Primers used to amplify the hypervariable 4 region (V4) of the 18S small subunit (SSU) ribosomal RNA (rRNA) gene (18S V4 hereafter) were based on published pan-eukaryotic sequences E572F and E1009R (33), which were modified to replace individual barcodes with overhang adapters (underlined) compatible with the Nextera library preparation system (Illumina, San Diego, CA, USA):

F
(SEQ ID NO: 66-SEQ ID NO: 15)
5′-TCGTCGGCAGCGTCAGATGTGTATAAGAGACAG-
CYGCGGTAATTCCAGCTC-3′
and
R
(SEQ ID NO: 67-SEQ ID NO: 16)
5′-GTCTCGTGGGCTCGGAGATGTGTATAAGAGACAG-
AYGGTATCTRATCRTCTTYG-3′.

The most commonly published HSR method (host amplification blocking), which we used in this study, was a peptide nucleic acid (PNA) mammal blocking primer (PNA Bio, Thousand Oaks, CA, USA): 5′-TCTTAATCATGGCCTCAGTT-3′ (SE ID NO: 17) (24). Conditions for amplicon PCR with and without blocking primer were based on those described in Mann et al. (24). Resulting PCR products were cleaned using AMPure XP beads (Agencourt, Beverley, MA, USA) according to manufacturer's instructions and 5 μl was used as template in a 25-μl PCR with the Illumina Nextera XT Index Kit v2 and limited-cycle PCR with an annealing temperature of 55° C. for 10 cycles. Indexed libraries were cleaned using Agencourt AMPure XP beads and quantified using a Qubit fluorometer (ThermoFisher Scientific, Waltham, MA, USA). Libraries were sequenced on an Illumina MiSeq instrument using paired-end 300×300 cycle V3 chemistry.

### Guide RNA Design and in Silico Screening

We used two concurrent approaches to design gRNA sequences to target vertebrate host 18S V4: 1) the ARB 7.0 software package (34) with the SILVA SSU rRNA 132 Non-redundant Reference (RefNR) database (35), and 2) The Broad Institute's online CRISPick tool (portals.broadinstitute.org/gppx/crispick/public) (36) using human (Homo sapiens, NCBI RefSeq GCF_000001405.40), house mouse (Mus musculus, NCBI RefSeq GCF_000001635.26), domestic dog (Canis lupus familiaris, NCBI RefSeq GCF_000002285.5), and chimpanzee (Pan troglodytes, NCBI RefSeq GCF_002880755.1) genomes as input. We screened 50 candidate gRNA sequences generated from each of these tools (n=100 total) using SILVA TestProbe (37) in silico hybridization to the SILVA 138.1 RefNR database with maximum stringency (no mismatches between gRNA sequence and DNA target) or allowing for a single mismatch outside of the 6-base pair “seed sequence” (Table 13). Resulting coverage metrics were used to choose the six gRNA sequences that targeted the highest number of vertebrates and lowest number of eukaryotic endosymbionts for further testing: arb321, arb326, arb615 were designed in the arb software suite, and CA149, CA172, PT7.1 were designed using CRISPick. Alignments of gRNAs with host sequences and digest maps were visualized using CLC Genomics Workbench v. 20.2.4 (Qiagen, Hilden, Germany).

### CRISPR-Cas9 In Vitro Digestion of Representative Organisms

All reagents for CC9 treatment of amplicons were components of the Alt-R CRISPR-Cas9 system (Integrated DNA Technologies, Coralville, IA, USA), based on recombinant Streptococcus pyogenes Cas9 nuclease, including Alt-R® S.p. Cas9 Nuclease V3, Alt-R® CRISPR-Cas9 tracrRNA, and Alt-R® CRISPR-Cas9 crRNA. crRNA is the component containing the specific targeting sequence that, when complexed with tracrRNA, forms the functional gRNA (see Table 13 for sequences). Digest reactions were performed following the IDT “Alt-R CRISPR-Cas9 system—in vitro cleavage of target DNA with RNP complex” protocol version 2.2 using recommendations for PCR product templates of 500-2000 base pair lengths and 2-5 nM final DNA concentration per reaction.

CC9 cleavage and gRNA specificity were initially assessed in vitro using a panel of genomic DNA samples extracted from single representative vertebrate hosts (n=5) and eukaryotic endosymbionts (n=6). Representative host organisms included: Mammal—Ursus maritimus (polar bear), Amphibian—Lithobates chiricahuensis (leopard frog), Bird—Gallus gallus (chicken), Reptile—Varanus varius (monitor lizard), and Fish—Salmo trutta (brown trout). Representative eukaryotic endosymbiont organisms included: Protozoan—Entamoeba histolytica (amoeba), Protozoan—Trypanosoma brucei (flagellate), Microsporidian—Encephalitozoon cuniculi, Acanthocephalan—Echinorhynchus salmonis (spiny-headed worm), Platyhelminth—Schistosoma mansoni (fluke), and Nematode—Ascaris suum (roundworm). 18S V4 amplicon PCR was performed as described above, and resulting amplicons were used in Alt-R CRISPR-Cas9 digest reactions. Cleavage products were separated by gel electrophoresis on 1.5% agarose gels containing 0.02 μg/ml ethidium bromide, visualized under ultraviolet light, and documented using a GelDoc XR imager (BioRad, Hercules, CA, USA). Successful cleavage was indicated by the presence of bands of between approximately 150-500 base pairs, which were discernably smaller than the full 18S V4 amplicon of approximately 700 base pairs.

### Comparison of Host Signal Reduction Methods

We compared the efficacy of HSR for improving eukaryotic endosymbiont metabarcoding by performing 18S V4 library preparation in conjunction with 4 different protocols 1) CC9 digestion of amplicons using gRNA arb321, 2) published V4 PNA mammal-blocking oligo described above [23] added to the amplicon PCR, 3) both CC9 digestion and PNA mammal-blocking oligo, and 4) mock-treated control (no CRISPR-Cas9 or PNA reagents added). PCR templates consisted of gDNA extracted from chimpanzee blood, liver, lung, colon, and fecal samples (n=3 each). 18S V4 library preparation and CC9 digests were performed as described above. For CC9 digested amplicons, uncleaved products (bands corresponding to undigested target amplicons) were excised from agarose gels using sterile razor blades and DNA was extracted from the gel matrix using a the ZymoClean Gel DNA Recovery Kit (Zymo, Irvine, CA, USA) according to manufacturer's instructions.

### Optimization of CRISPR-Cas9 Digest

We tested various ratios of ribonucleoprotein complex (RNP) to host target DNA of 0.75:1, 1:1, and 1.25:1 in our CRISPR-Cas9 digest. CC9 treatment was also tested at two steps in the metabarcoding protocol: 1) after initial amplification and cleanup, prior to indexing PCR (requiring one digest reaction per sample) or 2) after indexing PCR, clean up and pooling of libraries (requiring one digest reaction total for the combined pool of samples). For evaluation of the effect of gRNA targeting sequence on CC9 digest efficiency, we performed metabarcoding on chimpanzee blood samples (n=3) using a panel of all 6 newly designed gRNAs. We amplified 18S V4 from each sample and divided the PCR products into seven equal parts (one for each gRNA and one for a no-treatment control) prior to library preparation followed by sequencing and quantification of host read abundance under each condition. The top three gRNAs (arb326, CA149, PT7.1) were then tested in the same manner on a larger set of chimpanzee blood samples (n=31).

### Detection of Known Parasite Infections in Mammal Blood Samples

To test the effect of HSR and CC9 on detection of eukaryotic parasites in a verified infection, we performed eukaryotic endosymbiont metabarcoding on dog blood samples containing a mean of 57.8 Dirofilaria immitis microfilariae per 20 μl whole blood. We prepared sequencing libraries using CC9 digestion with a panel of all 6 newly designed gRNAs, amplification with a PNA blocking oligo or mock-treated control prior to sequencing, and quantified host read abundance under each condition.

For metabarcoding of naturally infected hosts, we used whole blood samples from wild red colobus that were characterized by microscopic investigation and PCR as part of a concluded study (32). Most samples (n=16 of 19) had been found to contain one of two distinct lineages of the apicomplexan parasite Hepatocystis: species A in 12 of 16 infected hosts, and species B in 4 of 16 infected hosts (32). We used aliquots of these same blood samples for gDNA extraction, 18S amplicon library preparation, treatment with CC9 digest or mock control, sequencing, and quantification of host read abundances.

### Sequence Data Processing and Analyses

Raw sequence reads were processed using QIIME2 v. 1.9.1 (38). Forward and reverse reads were assembled into paired contigs using the command multiple_join_paired_ends.py and quality filtered using the command multiple_split_libraries_fastq.py with default parameters, except for setting the Phred threshold to 30 or higher (−q 29) and minimum length to 100 bp (−1 100). Chimeras were identified with Usearch v. 6.1 (39) and removed. Reads were then assigned to OTUs using the QIIME protocol for open reference OTU picking with the command pick_open_reference_otus.py and the default UCLUST tool (v. 0.2.0) (40), and taxonomy was assigned to OTUs using default settings with the command assign_taxonomy.py against the SILVA database v. 132 (35). Still-undetermined OTUs were assigned using BLAST within QIIME2 (−m blast) against the full GenBank nucleotide database (41). OTUs constituting <0.5% of the total data set were removed from further analyses. Prism v. 8.4.3 (GraphPad Software, Inc.) was used for plotting data and conducting statistical analyses.

### REFERENCES

1. Forsman A M, Savage A E, Hoenig B D, Gaither M R. DNA Metabarcoding Across Disciplines: Sequencing Our Way to Greater Understanding Across Scales of Biological Organization. Integr Comp Biol. 2022; 62(2):191-8.
2. Hamady M, Knight R. Microbial community profiling for human microbiome projects: Tools, techniques, and challenges. Genome Res. 2009; 19(7):1141-52.
3. Tedersoo L, Bahram M, Zinger L, Nilsson R H, Kennedy P G, Yang T, et al. Best practices in metabarcoding of fungi: From experimental design to results. Mol Ecol. 2022; 31(10):2769-95.
4. Laforest-Lapointe I, Arrieta M C. Microbial Eukaryotes: a Missing Link in Gut Microbiome Studies. mSystems. 2018; 3(2).
5. Lundberg D S, Yourstone S, Mieczkowski P, Jones C D, Dangl J L. Practical innovations for high-throughput amplicon sequencing. Nat Methods. 2013; 10(10):999-1002.
6. Sakai M, Ikenaga M. Application of peptide nucleic acid (PNA)-PCR clamping technique to investigate the community structures of rhizobacteria associated with plant roots. J Microbiol Methods. 2013; 92(3):281-8.
7. Vestheim H, Jarman S N. Blocking primers to enhance PCR amplification of rare sequences in mixed samples—a case study on prey DNA in Antarctic krill stomachs. Front Zool. 2008; 5:12.
8. Belda E, Coulibaly B, Fofana A, Beavogui A H, Traore S F, Gohl D M, et al. Preferential suppression of Anopheles gambiae host sequences allows detection of the mosquito eukaryotic microbiome. Sci Rep. 2017; 7(1):3241.
9. Sow A, Brevault T, Benoit L, Chapuis M P, Galan M, Coeur d'acier A, et al. Deciphering host-parasitoid interactions and parasitism rates of crop pests using DNA metabarcoding. Sci Rep. 2019; 9(1):3646.
10. Nicolas L, Prina E, Lang T, Milon G. Real-time PCR for detection and quantitation of leishmania in mouse tissues. J Clin Microbiol. 2002; 40(5):1666-9.
11. Titus R G, Marchand M, Boon T, Louis J A. A limiting dilution assay for quantifying Leishmania major in tissues of infected mice. Parasite Immunol. 1985; 7(5):545-55.
12. Alberdi A, Aizpurua O, Gilbert M T P, Bohmann K. Scrutinizing key steps for reliable metabarcoding of environmental samples. Methods Ecol Evol. 2018; 9(1):134-47.
13. Jiang P, Lai S, Wu S, Zhao X M, Chen W H. Host DNA contents in fecal metagenomics as a biomarker for intestinal diseases and effective treatment. BMC Genomics. 2020; 21(1):348.
14. Feehery G R, Yigit E, Oyola S O, Langhorst B W, Schmidt V T, Stewart F J, et al. A method for selectively enriching microbial DNA from contaminating vertebrate host DNA. PLoS One. 2013; 8(10):e76096.
15. O'Rorke R, Lavery S, Jeffs A. PCR enrichment techniques to identify the diet of predators. Mol Ecol Resour. 2012; 12(1):5-17.
16. Flaherty B R, Talundzic E, Barratt J, Kines K J, Olsen C, Lane M, et al. Restriction enzyme digestion of host DNA enhances universal detection of parasitic pathogens in blood via targeted amplicon deep sequencing. Microbiome. 2018; 6(1):164.
17. Terahara T, Chow S, Kurogi H, Lee S H, Tsukamoto K, Mochioka N, et al. Efficiency of peptide nucleic acid-directed PCR clamping and its application in the investigation of natural diets of the Japanese eel leptocephali. PLoS One. 2011; 6(11):e25715.
18. Vestheim H, Deagle B E, Jarman S N. Application of blocking oligonucleotides to improve signal-to-noise ratio in a PCR. Methods Mol Biol. 2011; 687:265-74.
19. Mayer T, Mari A, Almario J, Murillo-Roos M, Abdullah M, Dombrowski N, et al. Obtaining deeper insights into microbiome diversity using a simple method to block host and non-targets in amplicon sequencing. bioRxiv. 2020.
20. Flaherty B R, Barratt J, Lane M, Talundzic E, Bradbury R S. Sensitive universal detection of blood parasites by selective pathogen-DNA enrichment and deep amplicon sequencing. Microbiome. 2021; 9(1):1.
21. Mamanova L, Coffey A J, Scott C E, Kozarewa I, Turner E H, Kumar A, et al. Target-enrichment strategies for next-generation sequencing. Nat Methods. 2010; 7(2):111-8.
22. von Wintzingerode F, Landt O, Ehrlich A, Gobel U B. Peptide nucleic acid-mediated PCR clamping as a useful supplement in the determination of microbial diversity. Appl Environ Microbiol. 2000; 66(2):549-57.
23. Troedsson C, Lee R F, Stokes V, Walters T L, Simonelli P, Frischer M E. Development of a denaturing high-performance liquid chromatography method for detection of protist parasites of metazoans. Appl Environ Microbiol. 2008; 74(14):4336-45.
24. Mann A E, Mazel F, Lemay M A, Morien E, Billy V, Kowalewski M, et al. Biodiversity of protists and nematodes in the wild nonhuman primate gut. Isme J. 2020; 14(2):609-22.
25. Hino A, Maruyama H, Kikuchi T. A novel method to assess the biodiversity of parasites using 18S rDNA Illumina sequencing; parasitome analysis method. Parasitol Int. 2016; 65(5):572-5.
26. Lappan R, Classon C, Kumar S, Singh O P, de Almeida R V, Chakravarty J, et al. Meta-taxonomic analysis of prokaryotic and eukaryotic gut flora in stool samples from visceral leishmaniasis cases and endemic controls in Bihar State India. PLoS Negl Trop Dis. 2019; 13(9):e0007444.
27. Wu J, Huang B, Chen H, Yin Q, Liu Y, Xiang Y, et al. The landscape of accessible chromatin in mammalian preimplantation embryos. Nature. 2016; 534(7609):652-7.
28. Gu W, Crawford E D, O'Donovan B D, Wilson M R, Chow E D, Retallack H, et al. Depletion of Abundant Sequences by Hybridization (DASH): using Cas9 to remove unwanted high-abundance species in sequencing libraries and molecular counting applications. Genome Biol. 2016; 17:41.
29. Song L, Xie K. Engineering CRISPR/Cas9 to mitigate abundant host contamination for 16S rRNA gene-based amplicon sequencing. Microbiome. 2020; 8(1):80.
30. Wu X, Kriz A J, Sharp P A. Target specificity of the CRISPR-Cas9 system. Quant Biol. 2014; 2(2):59-70.
31. Owens L A, Colitti B, Hirji I, Pizarro A, Jaffe J E, Moittie S, et al. A Sarcina bacterium linked to lethal disease in sanctuary chimpanzees in Sierra Leone. Nature communications. 2021; 12(1):763.
32. Thurber M I, Ghai R R, Hyeroba D, Weny G, Tumukunde A, Chapman C A, et al. Co-infection and cross-species transmission of divergent Hepatocystis lineages in a wild African primate community. Int J Parasitol. 2013; 43(8):613-9.
33. Comeau A M, Li W K, Tremblay J E, Carmack E C, Lovejoy C. Arctic Ocean microbial community structure before and after the 2007 record sea ice minimum. PLoS One. 2011; 6(11):e27492.
34. Ludwig W, Strunk O, Westram R, Richter L, Meier H, Yadhukumar, et al. ARB: a software environment for sequence data. Nucleic Acids Res. 2004; 32(4):1363-71.
35. Quast C, Pruesse E, Yilmaz P, Gerken J, Schweer T, Yarza P, et al. The SILVA ribosomal RNA gene database project: improved data processing and web-based tools. Nucleic Acids Res. 2013; 41 (Database issue):D590-6.
36. Doench J G, Fusi N, Sullender M, Hegde M, Vaimberg E W, Donovan K F, et al. Optimized gRNA design to maximize activity and minimize off-target effects of CRISPR-Cas9. Nat Biotechnol. 2016; 34(2):184-91.
37. Klindworth A, Pruesse E, Schweer T, Peplies J, Quast C, Horn M, et al. Evaluation of general 16S ribosomal RNA gene PCR primers for classical and next-generation sequencing-based diversity studies. Nucleic Acids Res. 2013; 41(1):el.
38. Caporaso J G, Kuczynski J, Stombaugh J, Bittinger K, Bushman F D, Costello E K, et al. QIIME allows analysis of high-throughput community sequencing data. Nat Methods. 2010; 7(5):335-6.
39. Edgar R C, Haas B J, Clemente J C, Quince C, Knight R. UCHIME improves sensitivity and speed of chimera detection. Bioinformatics. 2011; 27(16):2194-200.
40. Edgar R C. Search and clustering orders of magnitude faster than BLAST. Bioinformatics. 2010; 26(19):2460-1.
41. Sayers E W, Cavanaugh M, Clark K, Pruitt K D, Schoch C L, Sherry S T, et al. GenBank. Nucleic Acids Res. 2021; 49(D1):D92-D6.
42. Comeau A M, Douglas G M, Langille M G. Microbiome Helper: a Custom and Streamlined Workflow for Microbiome Research. mSystems. 2017; 2(1).
43. Cho S W, Kim S, Kim Y, Kweon J, Kim H S, Bae S, et al. Analysis of off-target effects of CRISPR/Cas-derived RNA-guided endonucleases and nickases. Genome Res. 2014; 24(1):132-41.
44. Bradley I M, Pinto A J, Guest J S. Design and Evaluation of Illumina MiSeq-Compatible, 18S rRNA Gene-Specific Primers for Improved Characterization of Mixed Phototrophic Communities. Appl Environ Microbiol. 2016; 82(19):5878-91.
45. Pinol J, Senar M A, Symondson W O C. The choice of universal primers and the characteristics of the species mixture determine when DNA metabarcoding can be quantitative. Mol Ecol. 2019; 28(2):407-19.

1. A primer set comprising a forward primer comprising a sequence selected from SEQ ID NOs: 1-4 and a reverse primer comprising a sequence selected from SEQ ID NOs: 5-8.

2. The primer set of claim 1, wherein the forward primer comprises SEQ ID NO: 3.

3. The primer set of claim 1, wherein the reverse primer comprises SEQ ID NO: 6.

4. A guide RNA (gRNA) selected from SEQ ID NOs: 9-14.

5. The gRNA of claim 4, wherein the gRNA hybridizes to 18S rRNA gene amplicons from vertebrates but does not hybridize to 18S rRNA gene amplicons from eukaryotic endosymbionts.

6. A mock community of eukaryotic endosymbionts comprising 18S rRNA genes, or portions thereof, from a plurality of eukaryotic endosymbionts in equimolar quantities, wherein the plurality of eukaryotic endosymbionts comprises two or more eukaryotic endosymbionts selected from the group consisting of: Echinorhynchus salmonis (ES201), Hymenolopis diminuta (H D1), Ascaris suum (AS1), Dirofilaria immitis (DI8), Trichinella spiralis (TS3), Encephalitozoon cuniculi (EC2), Entamoeba histolytica (EH3), Balamuthia mandrillaris (BM2), Naegleria fowleri (NF12), Leishmania major (LM4), Giardia intestinalis (GI405), Plasmodium falciparum: (PF115), Babesia sp. (Bab10), Toxoplasma gondii (TG3), Cryptosporidium hominis (CH109), and Blastocystis hominis 1 (ATCC 50177) (BH1).

7. The mock community of claim 6, wherein the plurality of eukaryotic endosymbionts comprises ES201, HD1, AS1, DI8, TS3, EC2, EH3, BM2, NF12, LM4, GI405, PF115, Bab10, TG3, CH109, and BH1.

8. A method for assessing the ability of a primer set to detect one or more eukaryotic endosymbionts, the method comprising:
a) amplifying the mock community of claim 6 using the primer set; and
b) detecting any resulting amplicons;
wherein detection of an amplicon associated with a particular eukaryotic endosymbiont indicates that the primer set is able to detect that particular eukaryotic endosymbiont.

9. A method for detecting one or more eukaryotic endosymbionts in a sample, the method comprising:
a) extracting DNA from the sample;
b) amplifying the DNA using the primer set of claim 1 to generate amplicons;
c) sequencing the amplicons to generate sequencing reads; and
d) analyzing the sequencing reads;
wherein the presence of sequencing reads associated with a particular eukaryotic endosymbiont indicates that the eukaryotic endosymbiont is present in the sample.

10. The method of claim 9, further comprising: adding an RNA-guided nuclease and a gRNA selected from SEQ ID NOs: 9-14 to the amplicons generated in step (b) to digest amplicons generated from host DNA prior to step (c).

11. A method for diagnosing and treating a subject with a parasitic infection, the method comprising:
a) obtaining a sample from the subject;
b) extracting DNA from the sample;
c) amplifying the DNA using the primer set of claim 1 to generate amplicons;
d) sequencing the amplicons to generate sequencing reads;
e) analyzing the sequencing reads to detect the presence of a parasite in the sample; and
f) treating the subject for the detected parasite.

12. The method of claim 11, wherein the subject is a human.

13. The method of claim 11, further comprising: adding an RNA-guided nuclease and a gRNA selected from SEQ ID NOs: 9-14 to the amplicons generated in step (c) to digest amplicons generated from host DNA prior to step (d).

14. The method of claim 9, further comprising amplifying a mock community in step (c) as a positive control.

15. The method of claim 9, wherein the sample is a blood sample or fecal sample.

16. The method of claim 15, wherein the primer set comprises the forward primer of SEQ ID NO: 3 and the reverse primer of SEQ ID NO: 6.

17. The method of claim 9, wherein the method is capable of detecting parasites from 24 clinically relevant clades.

18. The method of claim 9, wherein less than 50% of the sequencing reads are off-target reads.

19. A kit comprising the primer set of claim 1 and instructions for use.

20. The kit of claim 19, further comprising:
a) a gRNA selected from SEQ ID NOs: 9-14;
b) a mock community; and/or
c) adapters.

21. (canceled)

22. (canceled)

23. (canceled)


SEQUENCE LISTING (81 sequences)

SEQ ID NO: 1 - DNA, 19 bp, synthetic construct
  source 1..19: mol_type = other DNA
ctggtgccagcagccgcgg

SEQ ID NO: 2 - DNA, 18 bp, synthetic construct
  source 1..18: mol_type = other DNA
tggtgccagcagccgcgg

SEQ ID NO: 3 - DNA, 18 bp, synthetic construct
  source 1..18: mol_type = other DNA
agcagccgcggtaattcc

SEQ ID NO: 4 - DNA, 17 bp, synthetic construct
  source 1..17: mol_type = other DNA
tggtgccagcasccgcg

SEQ ID NO: 5 - DNA, 18 bp, synthetic construct
  source 1..18: mol_type = other DNA
  misc_difference 11: note = A, T, C, G, or I
tcaattycttnaastttc

SEQ ID NO: 6 - DNA, 22 bp, synthetic construct
  source 1..22: mol_type = other DNA
  misc_difference 15: note = A, T, C, G, or I
tccgtcaattycttnaastttc

SEQ ID NO: 7 - DNA, 23 bp, synthetic construct
  source 1..23: mol_type = other DNA
  misc_difference 16: note = A, T, C, G, or I
ttccgtcaattycttnaastttc

SEQ ID NO: 8 - DNA, 24 bp, synthetic construct
  source 1..24: mol_type = other DNA
  misc_difference 17: note = A, T, C, G, or I
cttccgtcaattycttnaastttc

SEQ ID NO: 9 - DNA, 20 bp, synthetic construct
  source 1..20: mol_type = other DNA
aactgaggccatgattaaga

SEQ ID NO: 10 - DNA, 19 bp, synthetic construct
  source 1..19: mol_type = other DNA
aggccatgattaagaggga

SEQ ID NO: 11 - DNA, 20 bp, synthetic construct
  source 1..20: mol_type = other DNA
gcagctaggaataatggaat

SEQ ID NO: 12 - DNA, 20 bp, synthetic construct
  source 1..20: mol_type = other DNA
attcttggaccggcgcaaga

SEQ ID NO: 13 - DNA, 20 bp, synthetic construct
  source 1..20: mol_type = other DNA
ctcagctaagagcatcgagg

SEQ ID NO: 14 - DNA, 20 bp, synthetic construct
  source 1..20: mol_type = other DNA
tcttagctgagtgtcccgcg

SEQ ID NO: 15 - DNA, 18 bp, synthetic construct
  source 1..18: mol_type = other DNA
cygcggtaattccagctc

SEQ ID NO: 16 - DNA, 20 bp, synthetic construct
  source 1..20: mol_type = other DNA
ayggtatctratcrtcttyg

SEQ ID NO: 17 - DNA, 20 bp, synthetic construct
  source 1..20: mol_type = other DNA
tcttaatcatggcctcagtt

SEQ ID NO: 18 - DNA, 19 bp, synthetic construct
  source 1..19: mol_type = other DNA
gtgccagcmgccgcggtaa

SEQ ID NO: 19 - DNA, 15 bp, synthetic construct
  source 1..15: mol_type = other DNA
ggtgcccttccgtca

SEQ ID NO: 20 - DNA, 15 bp, synthetic construct
  source 1..15: mol_type = other DNA
gtgccagcagccgcg

SEQ ID NO: 21 - DNA, 19 bp, synthetic construct
  source 1..19: mol_type = other DNA
tttaagtttcagccttgcg

SEQ ID NO: 22 - DNA, 20 bp, synthetic construct
  source 1..20: mol_type = other DNA
ccagcascygcggtaattcc

SEQ ID NO: 23 - DNA, 18 bp, synthetic construct
  source 1..18: mol_type = other DNA
actttcgttcttgatyra

SEQ ID NO: 24 - DNA, 15 bp, synthetic construct
  source 1..15: mol_type = other DNA
actttcgttcttgat

SEQ ID NO: 25 - DNA, 17 bp, synthetic construct
  source 1..17: mol_type = other DNA
ggcaagtctggtgccag

SEQ ID NO: 26 - DNA, 27 bp, synthetic construct
  source 1..27: mol_type = other DNA
gactacgacggtatctratcrtcttcg

SEQ ID NO: 27 - DNA, 20 bp, synthetic construct
  source 1..20: mol_type = other DNA
acggtatctratcrtcttcg

SEQ ID NO: 28 - DNA, 19 bp, synthetic construct
  source 1..19: mol_type = other DNA
gccgcggtaattccagctc

SEQ ID NO: 29 - DNA, 21 bp, synthetic construct
  source 1..21: mol_type = other DNA
atyyttggcaaatgctttcgc

SEQ ID NO: 30 - DNA, 20 bp, synthetic construct
  source 1..20: mol_type = other DNA
atacggtggtgtctgatcgc

SEQ ID NO: 31 - DNA, 19 bp, synthetic construct
  source 1..19: mol_type = other DNA
cagcagccgcggtaattcc

SEQ ID NO: 32 - DNA, 22 bp, synthetic construct
  source 1..22: mol_type = other DNA
cccgtgttgagtcaaattaagc

SEQ ID NO: 33 - DNA, 19 bp, synthetic construct
  source 1..19: mol_type = other DNA
gcggtaattccagctccaa

SEQ ID NO: 34 - DNA, 17 bp, synthetic construct
  source 1..17: mol_type = other DNA
ttggcaaatgctttcgc

SEQ ID NO: 35 - DNA, 18 bp, synthetic construct
  source 1..18: mol_type = other DNA
ttaaarvgytcgtagtyg

SEQ ID NO: 36 - DNA, 17 bp, synthetic construct
  source 1..17: mol_type = other DNA
cggtaaytccagctcyv

SEQ ID NO: 37 - DNA, 18 bp, synthetic construct
  source 1..18: mol_type = other DNA
gccagcavcygcggtaay

SEQ ID NO: 38 - DNA, 18 bp, synthetic construct
  source 1..18: mol_type = other DNA
ccgtcaatthcttyaart

SEQ ID NO: 39 - DNA, 20 bp, synthetic construct
  source 1..20: mol_type = other DNA
gccagcagccgcggtaattc

SEQ ID NO: 40 - DNA, 25 bp, synthetic construct
  source 1..25: mol_type = other DNA
acattcttggcaaatgctttcgcag

SEQ ID NO: 41 - DNA, 20 bp, synthetic construct
  source 1..20: mol_type = other DNA
agccgcggtaattccagctc

SEQ ID NO: 42 - DNA, 20 bp, synthetic construct
  source 1..20: mol_type = other DNA
ggtggtgcccttccgtcaat

SEQ ID NO: 43 - DNA, 20 bp, synthetic construct
  source 1..20: mol_type = other DNA
tggagggcaagtctggtgcc

SEQ ID NO: 44 - DNA, 26 bp, synthetic construct
  source 1..26: mol_type = other DNA
tacggtatctgatcgtcttcgatccc

SEQ ID NO: 45 - DNA, 22 bp, synthetic construct
  source 1..22: mol_type = other DNA
ctggtgccagcagccgcggyaa

SEQ ID NO: 46 - DNA, 20 bp, synthetic construct
  source 1..20: mol_type = other DNA
tccgtcaattyctttaagtt

SEQ ID NO: 47 - DNA, 19 bp, synthetic construct
  source 1..19: mol_type = other DNA
ggtgccagcagccgcggta

SEQ ID NO: 48 - DNA, 19 bp, synthetic construct
  source 1..19: mol_type = other DNA
ctttaagtttcagctttgc

SEQ ID NO: 49 - DNA, 24 bp, synthetic construct
  source 1..24: mol_type = other DNA
cagggcaagtctggtgccagcagc

SEQ ID NO: 50 - DNA, 23 bp, synthetic construct
  source 1..23: mol_type = other DNA
gactttcgttcttgattaatgaa

SEQ ID NO: 51 - DNA, 21 bp, synthetic construct
  source 1..21: mol_type = other DNA
aacctggttgatcctgccagt

SEQ ID NO: 52 - DNA, 24 bp, synthetic construct
  source 1..24: mol_type = other DNA
tgatccttctgcaggttcacctac

SEQ ID NO: 53 - DNA, 16 bp, synthetic construct
  source 1..16: mol_type = other DNA
cygcaggttcacctac

SEQ ID NO: 54 - DNA, 18 bp, synthetic construct
  source 1..18: mol_type = other DNA
ccggagagrgagcmtkag

SEQ ID NO: 55 - DNA, 20 bp, synthetic construct
  source 1..20: mol_type = other DNA
ccttccgcaggttcacctac

SEQ ID NO: 56 - DNA, 21 bp, synthetic construct
  source 1..21: mol_type = other DNA
ctggttgatcctgccagtakt

SEQ ID NO: 57 - DNA, 18 bp, synthetic construct
  source 1..18: mol_type = other DNA
ctggttgatcctgccagt

SEQ ID NO: 58 - DNA, 23 bp, synthetic construct
  source 1..23: mol_type = other DNA
cgcgaanggctcattanawcagc

SEQ ID NO: 59 - DNA, 19 bp, synthetic construct
  source 1..19: mol_type = other DNA
acggctcaggacaacggtt

SEQ ID NO: 60 - DNA, 24 bp, synthetic construct
  source 1..24: mol_type = other DNA
ggttcacctacgganaccttgtta

SEQ ID NO: 61 - DNA, 19 bp, synthetic construct
  source 1..19: mol_type = other DNA
tcgtcttctcagcgccggt

SEQ ID NO: 62 - DNA, 22 bp, synthetic construct
  source 1..22: mol_type = other DNA
gattaagccatgcatgtstaag

SEQ ID NO: 63 - DNA, 17 bp, synthetic construct
  source 1..17: mol_type = other DNA
ggttcgactccggagag

SEQ ID NO: 64 - DNA, 20 bp, synthetic construct
  source 1..20: mol_type = other DNA
tggataactgtaatractct

SEQ ID NO: 65 - DNA, 22 bp, synthetic construct
  source 1..22: mol_type = other DNA
gacctyactaaaccattcaatc

SEQ ID NO: 66 - DNA, 33 bp, synthetic construct
  source 1..33: mol_type = other DNA
tcgtcggcagcgtcagatgtgtataagagacag

SEQ ID NO: 67 - DNA, 34 bp, synthetic construct
  source 1..34: mol_type = other DNA
gtctcgtgggctcggagatgtgtataagagacag

SEQ ID NO: 68 - DNA, 20 bp, Trichinella pseudospiralis
  source 1..20: mol_type = genomic DNA
accggagataagtattgaaa

SEQ ID NO: 69 - DNA, 19 bp, Trichinella pseudospiralis
  source 1..19: mol_type = genomic DNA
agataagtattgaaaggaa

SEQ ID NO: 70 - DNA, 20 bp, Trichinella pseudospiralis
  source 1..20: mol_type = genomic DNA
ggtgcatggaataatagaat

SEQ ID NO: 71 - DNA, 20 bp, Trichinella pseudospiralis
  source 1..20: mol_type = genomic DNA
attcttggatcgcagcaaga

SEQ ID NO: 72 - DNA, 18 bp, synthetic construct
  source 1..18: mol_type = other DNA
tggtgccagcagccgcgg

SEQ ID NO: 73 - DNA, 26 bp, synthetic construct
  source 1..26: mol_type = other DNA
ctggtgccagcagccgcggtaattcc

SEQ ID NO: 74 - DNA, 18 bp, synthetic construct
  source 1..18: mol_type = other DNA
gaaasttcaagraattga

SEQ ID NO: 75 - DNA, 22 bp, synthetic construct
  source 1..22: mol_type = other DNA
gaaasttcaagraattgacgga

SEQ ID NO: 76 - DNA, 23 bp, synthetic construct
  source 1..23: mol_type = other DNA
gaaasttcaagraattgacggaa

SEQ ID NO: 77 - DNA, 24 bp, synthetic construct
  source 1..24: mol_type = other DNA
gaaasttcaagraattgacggaag

SEQ ID NO: 78 - DNA, 23 bp, synthetic construct
  source 1..23: mol_type = other DNA
gaaanttcaagnaattgacggaa

SEQ ID NO: 79 - DNA, 22 bp, synthetic construct
  source 1..22: mol_type = other DNA
gaaattcaagaattgacggaag

SEQ ID NO: 80 - DNA, 16 bp, synthetic construct
  source 1..16: mol_type = other DNA
gtacacaccgcccgtc

SEQ ID NO: 81 - DNA, 24 bp, synthetic construct
  source 1..24: mol_type = other DNA
tgatccttctgcaggttcacctac